(19) United States
(12) Patent Application Publication
(10) Pub. No.: US 2012/0196853 A1
(43) Pub. Date: Aug. 2, 2012

(54) Novel Quinoline-Hepcidine Antagonists

(75) Inventors: Franz Dürrenberger, Dornach (CH); Susanna Burckhardt, Zurich (CH); Peter O. Geisser, St. Gallen (CH); Wilm Buhr, Konstanz (DE); Felix Funk, Winterthur (CH); Vincent A. Corden, Stanford in the Vale (GB); Tara Davenport, Abingdon (GB); Stefan Jaeger, Hamburg (DE); Mark Slack, Hamburg (DE); Christopher J. Yarnold, Didcot Oxon (GB); Wei Tsung Yau, Didcot Oxon (GB); Stephen M. Courtney, Stanford in the Vale (GB)

(73) Assignee: VIFOR (INTERNATIONAL) AG, St. Gallen (CH)

(21) Appl. No.: 13/390,785

(22) PCT Filed: Aug. 19, 2010
(86) PCT No.: PCT/EP10/62117

§ 371 Date: Apr. 18, 2012

(30) Foreign Application Priority Data
Aug. 20, 2009 (EP) 09168255.9

(51) Int. Cl. A61K 31/5377; A61K 31/496; A61P 3/00; A61P 7/06; A61P 19/02; A61P 13/12; A61P 9/00; A61P 29/00; A61P 19/04; A61P 1/00; A61K 31/4709; A61P 35/00

(52) U.S. Cl. 514/232.5; 514/235.2; 514/314; 514/253.06

(57) ABSTRACT

The present invention relates to novel hepcidin antagonists of the general formula (I), pharmaceutical compositions comprising them and the use thereof as medicaments, in particular for treatment of disorders in iron metabolism, such as, in particular, iron deficiency diseases and anaemias, in particular anaemias in connection with chronic inflammatory diseases (ACD: anaemia of chronic disease and AI: anaemia of inflammation).

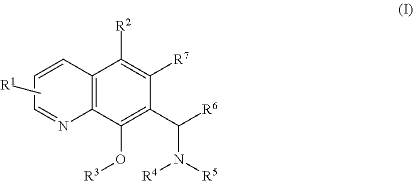

### INTRODUCTION

The invention relates to novel hepcidin antagonists of the general formula (I), pharmaceutical compositions comprising these and their use for treatment of iron metabolism disorders, in particular of anaemias in connection with chronic inflammatory diseases (anaemia of chronic disease (ACD) and anaemia of inflammation (AI)) or of iron deficiency symptoms and iron deficiency anaemias.

### BACKGROUND

Iron is an essential trace element for almost all organisms and in this context is relevant in particular for growth and blood formation. The balance of iron metabolism in this context is primarily regulated at the level of recovery of iron from haemoglobin from ageing erythrocytes and duodenal absorption of iron bonded in food. The iron released is absorbed via the intestine, in particular by way of specific transport systems (DMT-1, ferroportin, transferrin, transferrin receptors), transported into the blood stream and passed on by this means into the corresponding tissue and organs.

The element iron is of great importance in the human body inter alia for oxygen transport, oxygen uptake, cell functions, such as mitochondrial electron transport, and finally for energy metabolism in total.

The body of a human contains on average 4 to 5 g of iron, this being present in enzymes, in haemoglobin and myoglobin and as depot or reserve iron in the form of ferritin and haemosiderin.

About half of this iron, approx. 2 g, is present as haem iron bonded in the haemoglobin of red blood corpuscles. Since these erythrocytes have only a limited life (75-150 days), new ones must constantly be formed and old ones eliminated (over 2 million new erythrocytes are formed per second). This high regeneration capacity is achieved by macrophages, in that these absorb the ageing erythrocytes by phagocytosis, lyse them and in this way can recycle the iron contained in them for the iron metabolism. The amount of iron required daily for erythropoiesis of approx. 25 mg is thus mostly provided.

The daily iron requirement of an adult human is between 0.5 and 1.5 mg per day, and for infants and women in pregnancy the iron requirement is 2 to 5 mg per day. Daily iron losses, e.g. by exfoliation of skin cells and epithelial cells, is comparatively low, but increased iron losses occur, for example, in women during menstrual bleeding. Blood losses generally can considerably reduce iron metabolism, since about 1 mg of iron is lost per 2 ml of blood. The normal daily iron loss of approx. 1 mg is conventionally replaced again by an adult, healthy human via the daily food intake. Iron metabolism is regulated via absorption, the absorption rate of the iron present in food being between 6 and 12%, and in the event of iron deficiency the absorption rate is up to 25%. The absorption rate is regulated by the organism as a function of iron requirement and the size of the iron store. In this context, the human organism uses both divalent and trivalent iron ions. Iron(III) compounds are conventionally dissolved in the stomach at a sufficiently acid pH and are thus made available for absorption. Absorption of the iron takes place in the upper small intestine by mucosa cells. In this context, for absorption trivalent non-haem iron is first reduced to Fe²⁺ e.g. by ferrireductase (duodenal cytochrome b at the membrane) in the membrane of intestinal cells, so that it can then be transported by the transport protein DMT1 (divalent metal transporter 1) into the intestinal cells. On the other hand, haem iron enters into the enterocytes unchanged via the cell membrane. In the enterocytes, iron is either stored as depot iron in ferritin or released into the blood by the transport protein ferroportin, bonded to transferrin. Hepcidin plays a central role in this operation, since it is the essential regulation factor of iron uptake. The divalent iron transported into the blood by the ferroportin is converted into trivalent iron by oxidases (ceruloplasmin, hephaestin), which is then transported to the relevant places in the organism by means of transferrin (see for example: “Balancing acts: molecular control of mammalian iron metabolism”. M. W. Hentze, Cell 117, 2004, 285-297.)

The regulation of the iron level in this context is controlled or regulated by hepcidin.

Hepcidin is a peptide hormone which is produced in the liver. The prevailing active form has 25 amino acids (see for example: “Hepcidin, a key regulator of iron metabolism and mediator of anemia of inflammation”. T. Ganz Blood 102, 2003, 783-8), although two forms shortened at the amino end, hepcidin-22 and hepcidin-20, have been found. Hepcidin acts on iron uptake via the intestine, via the placenta and on the release of iron from the reticuloendothelial system. In the body, hepcidin is synthesized from so-called pro-hepcidin in the liver, pro-hepcidin being coded by the so-called HAMP gene. If the organism is adequately supplied with iron and oxygen, increased hepcidin is formed. In the mucosa cells of the small intestine and in the macrophages, hepcidin binds to ferroportin, by means of which iron is conventionally transported out of the cell interior into the blood.

The transport protein ferroportin is a membrane transport protein comprising 571 amino acids which is formed and located in the liver, spleen, kidneys, heart, intestine and placenta. In particular, in this context ferroportin is located in the basolateral membrane of intestinal epithelial cells. The ferroportin bound in this way effects export of iron into the blood here. In this context, ferroportin very probably transports iron as Fe²⁺. If hepcidin is bound to ferroportin, ferroportin is transported into the cell interior and degraded, as a result of which the release of iron from the cells is then almost completely blocked. If the ferroportin is inactivated via hepcidin, the iron stored in the mucosa cells therefore cannot be transported away, and the iron is lost with the natural exfoliation of cells via the stool. As a result, absorption of iron in the intestine is reduced by hepcidin. On the other hand, if the iron content in the serum is lowered, hepcidin production in the hepatocytes of the liver is reduced, so that less hepcidin is released and therefore less ferroportin is inactivated, as a result of which an increased amount of iron can be transported into the serum.

Ferroportin is moreover located to a high degree in the reticuloendothelial system (RES), to which the macrophages also belong.

Hepcidin plays an important role here in the event of impaired iron metabolism in the context of chronic inflammations, since interleukin-6 in particular is increased with such inflammations, which leads to an increase in the hepcidin level. Increased hepcidin is bound to the ferroportin of the macrophages by this means, as a result of which release of iron is blocked here, which in the end then leads to an inflammation-related anaemia (ACD or AI).

Since the organism of mammals cannot actively excrete iron, iron metabolism is essentially controlled via cellular release of iron from macrophages, hepatocytes and enterocytes by way of hepcidin.

Hepcidin thus plays an important role in functional anaemia. In this case, in spite of a full iron store, the iron requirement of bone marrow for erythropoiesis is not met sufficiently. The reason for this is assumed to be an increased hepcidin concentration, which in particular limits the transport of iron from the macrophages by blocking the ferroportin and thus greatly reduces the release of iron recycled by phagocytosis.

In the event of a disturbance in the hepcidin regulation mechanism, a direct effect thus manifests itself on iron metabolism in the organism. For example, if hepcidin expression is prevented, for example by a genetic defect, this leads directly to an overloading of iron, which is known as the iron storage disease haemochromatosis.

On the other hand, overexpression of hepcidin, for example due to inflammation processes, for example with chronic inflammations, results directly in reduced serum iron levels. In pathological cases this can lead to a reduced content of haemoglobin, reduced erythrocyte production and therefore to an anaemia.

The duration of use of chemotherapeutics in carcinoma treatments can be significantly reduced by an existing anaemia, since the state of reduced formation of red blood corpuscles caused by the chemotherapeutics employed is intensified still further by an existing anaemia.

Further symptoms of anaemias include tiredness, pallor and reduced attention capacities. The clinical symptoms of anaemia include low serum iron contents (hypoferraemia), low haemoglobin contents, low haematocrit level and a reduced number of red blood corpuscles, reduced reticulocytes and increased values of soluble transferrin receptors.

Iron deficiency symptoms or iron anaemias are conventionally treated by supplying iron. In this context, substitution with iron takes place either by the oral route or by intravenous administration of iron. Erythropoietin and other erythropoiesis-stimulating substances can moreover also be employed in the treatment of anaemias to give a boost to the formation of red blood corpuscles.

Anaemias which are caused by chronic diseases, e.g. chronic inflammatory diseases, can be treated only inadequately with such conventional treatment methods. Cytokines, such as in particular inflammatory cytokine, in particular play a particular role in anaemias which are based on chronic inflammation processes. An overexpression of hepcidin occurs in particular with such chronic inflammatory diseases and is known to lead to a reduced availability of iron for the formation of the red blood corpuscles.

From this emerges the need for an effective treatment method for hepcidin-mediated or -imparted anaemias, in particular those which cannot be treated with conventional iron substitution, such as those anaemias which are caused by chronic inflammatory diseases (ACD and AI).

Anaemia is to be attributed inter alia to those chronic inflammatory diseases mentioned, and to malnutrition or low-iron diets or unbalanced, low-iron eating habits. Anaemias moreover occur due to reduced or poor absorption of iron, for example due to gastrectomies or diseases such as Crohn's disease. An iron deficiency can also occur as a result of an increased blood loss, e.g. due to an injury, heavy menstrual bleeding or blood donation. An increased iron requirement in the growth phase of adolescents and children and in pregnant women is also known. Since an iron deficiency leads not only to a reduced formation of red blood corpuscles but therefore also to a poor supply of oxygen to the organism, which can lead to the abovementioned symptoms, such as tiredness, pallor and lack of concentration and also precisely in adolescents to long-term negative effects on cognitive development, a particularly effective therapy in addition to the known conventional substitution therapy is also of particular interest for this sector.

Compounds which bind to hepcidin or to ferroportin and therefore inhibit the binding of hepcidin to ferroportin and therefore in turn prevent the inactivation of ferroportin by hepcidin, or compounds which, although hepcidin is bound to ferroportin, prevent the internalization of the hepcidin-ferroportin complex, and in this manner prevent the inactivation of the ferroportin by the hepcidin, can be called in general terms hepcidin antagonists.

By using such hepcidin antagonists, there is moreover also generally the possibility, for example by inhibiting hepcidin expression or by blocking the hepcidin-ferroportin interaction, of acting directly on the regulation mechanism of hepcidin and therefore of preventing via this route blocking of the iron transport pathway from tissue macrophages, liver cells and mucosa cells into the serum via the transport protein ferroportin. With such hepcidin antagonists or ferroportin expression inhibitors, substances are therefore available which are suitable for the preparation of pharmaceutical compositions or medicaments in the treatment of anaemias, in particular anaemias with chronic inflammatory diseases. These substances can be employed for treatment of such disorders and the resulting diseases, since these have a direct influence on the increase in the release of recycled haem iron by macrophages and effect an increase in the iron absorption of iron released from food in the intestinal tract. Such substances, inhibitors of hepcidin expression or hepcidin antagonists, can therefore be used for treatment of iron metabolism disorders, such as iron deficiency diseases, anaemias and anaemia-related diseases. In particular, this also includes those anaemias which are caused by acute or chronic inflammatory diseases, such as, for example, osteoarticular diseases, such as rheumatoid polyarthritis, or diseases which are associated with inflammatory syndromes. Such substances can therefore be of particular benefit in particular in the indications of cancer, in particular colorectal cancer, multiple myeloma, ovarian and endometrial cancer and prostate cancer, CKD 3-5 (chronic kidney disease stage 3-5) CHF (chronic heart failure), RA (rheumatoid arthritis), SLE (systemic lupus erythematosus) and IBD (inflammatory bowel disease).

### PRIOR ART

Hepcidin antagonists or compounds which have an inhibiting or assisting action on the biochemical regulation pathways in iron metabolism are known in principle from the prior art.

Thus, for example, WO 2008/036933 describes double-stranded dsRNA which has an inhibiting action on the expression of human HAMP genes in cells and therefore already suppresses the formation of hepcidin, which is coded by the HAMP gene, at a very early stage in the iron metabolism signal pathway. As a result, less hepcidin is formed, so that hepcidin is not available for the inhibition of ferroportin, so that the transport of iron from the cell into the blood by ferroportin can take place unimpeded.

Further compounds which aim directly at reduction of hepcidin expression are known from US 2005/020487, which describes compounds which have an HIF-α stabilizing action and therefore lead to a reduction in hepcidin expression.

The subject matter of US 2007/004618 is siRNA, which has a directly inhibiting action on hepcidin mRNA expression.

All these compounds or methods are therefore those which start in the iron metabolism pathway before formation of the hepcidin and already regulate its general formation downwards. In addition, however, such substances and compounds are also known and described in the prior art which bind in the body to hepcidin which has already formed and therefore inhibit its binding action on the membrane transport protein ferroportin, so that an inactivation of ferroportin by hepcidin is no longer possible. Such compounds are therefore so-called hepcidin antagonists, those based on hepcidin antibodies being known in particular from this group. Such documents are furthermore known in the prior art which describe various mechanisms for action on hepcidin expression, for example by antisense RNA or DNA molecules, ribozymes and anti-hepcidin antibodies. Such mechanisms are described, for example, in EP 1 392 345.

WO09/058,797 furthermore discloses anti-hepcidin antibodies and the use thereof for specific binding to human hepcidin-25, and therefore the use thereof for therapeutic treatment of low iron contents, in particular of anaemias.

Further compounds which act as hepcidin antagonists and are formed from the group of hepcidin antibodies are known from EP 1 578 254, WO08/097,461, US2006/019339, WO09/044,284 or WO09/027,752.

In addition, antibodies which bind to ferroportin-1 and therefore activate ferroportin in order to assist in the iron transport from the cell into the serum by this means are also known. Such ferroportin-1 antibodies are known, for example, from US2007/218055.

All these compounds described which can act as hepcidin antagonists or can display an inhibiting action in hepcidin expression are higher molecular weight compounds, in particular those which are chiefly obtainable by genetic engineering processes.

In addition, low molecular weight compounds which play a role in iron metabolism and which can have either an inhibiting or also an assisting action are also known.

WO08/109,840 thus describes certain tricyclic compounds which can be employed in particular for treatment of disorders in iron metabolism, such as, for example, ferroportin disorders, these compounds being able to act by regulation of DMT-1 in the form of inhibition or activation. In this context, the compounds of this WO08/109,840 are described in particular as DMT-1 inhibitors, whereby they can preferably be employed on diseases with increased iron accumulation or iron storage diseases, such as haemochromatosis.

WO08/121,861 also discloses low molecular weight compounds which have a regulating action on the DMT-1 mechanism. Certain pyrazole and pyrrole compounds are dealt with here, treatment of iron overloading disorders, for example on the basis of ferroportin disorders, also being described here in particular.

The subject matter of US2008/234384 is furthermore certain diaryl and diheteroaryl compounds for treatment of disorders in iron metabolism, such as, for example, ferroportin disorders, which likewise by their action as DMT-1 inhibitors can be employed in particular for treatment of disorders on the basis of increased iron accumulation. In this document, however, possible DMT-1 regulatory mechanisms which can be employed for use on iron deficiency symptoms are also mentioned quite generally.

The same applies to WO08/151,288, which describes certain aromatic and heteroaromatic compounds with an action on DMT-1 regulation and therefore for treatment of disorders in iron metabolism.

The low molecular weight compounds described in the prior art which have an action on iron metabolism are therefore based on DMT-1 regulatory mechanisms and are disclosed in particular for use as agents for treatment of iron accumulation disorders or iron overloading syndromes, such as haemochromatosis.

“Hepcidin—Central-regulator of iron-metabolism” (Atanasiu Valeriu et al., European Journal of Haematology, 78 (1), 2007) gives an overview of hepcidin and its function. However, no indications of low molecular weight antagonists, in particular those with a quinoline structure, emerge from this.

Chemical compounds on the structural basis of quinolines thus have not yet hitherto been described in connection with treatment of disorders in iron metabolism. Furthermore, no low molecular weight chemical structures which display their action as hepcidin antagonists and as a result are suitable for treatment of disorders in iron metabolism have yet been described hitherto.

The present invention also provides novel quinoline compounds of the general structural formula (I) according to the present invention.

US 2009/0053192 A1 discloses tissue-nonspecific alkaline phosphatase (TNAP) activators and the use thereof for bone mineralization, in particular in the treatment of hypophosphatasia and osteoporosis. The group of TNAP activators disclosed therein also includes in particular the quinoline compound 7-(morpholin-4-yl-pyridin-2-yl-methyl)-quinolin-8-ol, corresponding to Example Compound 1 of the present invention. However, an action in the treatment of disorders in iron metabolism does not emerge from this.

“Identification and Biochemical Characterization of Small-Molecule Inhibitors of Clostridium botulinum Neurotoxin Serotype A” (Roxas-Duncan et al., Antimicrobial Agents and Chemotherapy; 08/2009) furthermore discloses selected low molecular weight compounds, also including in particular some selected quinoline compounds for treatment of botulism. From here also no indication of an action of such quinoline compounds in the treatment of disorders in iron metabolism emerges.

In “The reaction of aldehydes and aromatic amines with 8-quinolinol” (Phillips et al.; Journal of the American Chemical Society, 75, 1953), the reaction of 8-quinolinol with aldehydes and aromatic amines to form specific arylamine-substituted 8-quinolinols is described. However, an action of the specific quinoline compounds disclosed therein for medical use, in particular in the treatment of disorders in iron metabolism, is not mentioned therein.

In “Antiamebic Agents. III. Basic derivatives of Chloro-8-quinolinols (Burckhalter et al.; Journal of the American Chemical Society, 76, 1954), the anti-amoeba action of selected specific quinoline compounds, including also Example Compound 31 of the present invention, is described, and the use thereof as an antiparasitic agent. An indication of an action of such selected quinoline compounds in the treatment of disorders in iron metabolism also does not emerge therefrom.

In “Synthesis of some 7-substituted 8-hydroxyquinoline derivatives of thiazoles & oxazoles as potential pesticides” (Nath et al., Indian Journal of Chemistry, vol. 20B, no. 7, 1981), the reaction of 8-quinolinol with benzaldehyde and aromatic amines to form specific thiazole- and oxazolamine-substituted 8-quinolinols and the action thereof as pesticides with a fungicidal and antibacterial action is described. A medical use of the specific quinoline compounds disclosed therein or in particular an action in the treatment of disorders in iron metabolism does not emerge therefrom.

In “High-throughput screen for novel antimicrobials using a whole animal infection model” (Moy et al., ACS Chemical Biology, 4 (7), 06/2009), novel compounds with an antimicrobial action and the medical use thereof are described, one of the active substances being 5-chloro-7-[3-fluorophenyl-(pyridin-2-ylamino)-methyl]-quinolin-8-ol. An indication of an action in the treatment of disorders in iron metabolism also does not emerge therefrom.

In “Synthesis and cytotoxicity evaluation of some 8-hydroxyquinoline derivatives” (Shen Ai-Yu et al., Journal of Pharmacy and Pharmacology, 51 (5), 1999), the synthesis of 7-pyrrolidinemethyl-8-hydroxyquinoline, 7-morpholinemethyl-8-hydroxyquinoline (corresponding to Example Compound 22 of the present invention), 7-piperidinemethyl-8-hydroxyquinoline and 7-diethylaminomethyl-8-hydroxyquinoline by reaction of 8-hydroxyquinoline with secondary amines and formaldehyde and the action of these compounds as anticancer agents are described. An indication of an action in the treatment of disorders in iron metabolism also does not emerge therefrom.

### OBJECT

The object of the present invention was to provide in particular such compounds which can be employed for use for iron deficiency disorders or anaemias, in particular ACD and AI, and which act in iron metabolism in particular as hepcidin antagonists and therefore display an antagonistic and via this a regulating action in the hepcidin-ferroportin interaction in iron metabolism. It was furthermore in particular an object of the present invention to provide in this context such compounds which are chosen from the group of low molecular weight compounds and which generally can be prepared by simpler synthesis routes than the antagonistic or hepcidin-inhibiting compounds obtainable by genetic engineering processes, such as RNA, DNA or antibodies.

### DESCRIPTION OF THE INVENTION

The inventors have found that certain compounds from the group of quinolines have an action as hepcidin antagonists.

The invention provides compounds of the general structural formula (I)

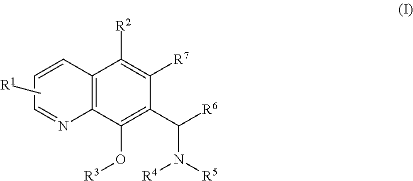

wherein
R¹, R² and R⁷ are identical or different and are each chosen from the group consisting of:
hydrogen,
hydroxyl,
halogen,
cyano,
nitro,
carboxyl,
sulfonic acid radical (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heteroaryl;
   R⁶ is chosen from the group consisting of:
hydrogen,
optionally substituted alkyl,
optionally substituted alkenyl,
optionally substituted alkynyl,
optionally substituted aryl,
optionally substituted heteroaryl;
   R³ is chosen from the group consisting of:
hydrogen,
optionally substituted alkyl,
optionally substituted alkenyl,
optionally substituted alkynyl,
optionally substituted acyl,
optionally substituted aryl,
optionally substituted heteroaryl; and
   R⁴ and R⁵ are identical or different and are each chosen from the group consisting of:
hydrogen,
optionally substituted alkyl,
optionally substituted alkenyl,
optionally substituted alkynyl,
optionally substituted acyl,
optionally substituted aryl,
optionally substituted heteroaryl, or
wherein R⁴ and R⁵ together with the nitrogen atom to which they are bonded form a saturated or unsaturated, optionally substituted 5- to 8-membered ring which can optionally contain further hetero atoms;
   or pharmaceutically acceptable salts thereof.

In the context of the overall invention, the abovementioned substituent groups are defined as follows:

Optionally substituted alkyl preferably includes:

straight-chain or branched alkyl having 1 to 8, preferably 1 to 6, particularly preferably 1 to 4 carbon atoms, cycloalkyl having 3 to 8, preferably 5 or 6 carbon atoms, which can optionally in each case carry preferably 1 to 3 identical or different substituents which, for example, are chosen from the group consisting of: hydroxyl, halogen, cyano, alkoxy, as defined below, carboxyl, acyl, as defined below, cycloalkyl, as defined below, aryl, as defined below, heteroaryl, as defined below, amino, and mercapto. In this context, halogen here and in the context of the present invention includes fluorine, chlorine, bromine and iodine, preferably fluorine or chlorine. Furthermore, one or more, more preferably 1 to 3 carbon atoms can be replaced by hetero-analogous groups which contain nitrogen, oxygen or sulfur. This means in particular that, for example, one or more methylene groups in the alkyl radicals can be replaced by NH, O or S. Furthermore, one or more H atoms of a methyl group, preferably 1 to 3 H atoms, can be replaced by fluorine.

Examples of alkyl radicals having 1 to 8 carbon atoms include: a methyl group, an ethyl group, an n-propyl group, an i-propyl group, an n-butyl group, an i-butyl group, a sec-butyl group, a t-butyl group, an n-pentyl group, an i-pentyl group, a sec-pentyl group, a t-pentyl group, a 2-methylbutyl group, an n-hexyl group, a 1-methylpentyl group, a 2-methylpentyl group, a 3-methylpentyl group, a 4-methylpentyl group, a 1-ethylbutyl group, a 2-ethylbutyl group, a 3-ethylbutyl group, a 1,1-dimethylbutyl group, a 2,2-dimethylbutyl group, a 3,3-dimethylbutyl group, a 1-ethyl-1-methylpropyl group, an n-heptyl group, a 1-methylhexyl group, a 2-methylhexyl group, a 3-methylhexyl group, a 4-methylhexyl group, a 5-methylhexyl group, a 1-ethylpentyl group, a 2-ethylpentyl group, a 3-ethylpentyl group, a 4-ethylpentyl group, a 1,1-dimethylpentyl group, a 2,2-dimethylpentyl group, a 3,3-dimethylpentyl group, a 4,4-dimethylpentyl group, a 1-propylbutyl group, an n-octyl group, a 1-methylheptyl group, a 2-methylheptyl group, a 3-methylheptyl group, a 4-methylheptyl group, a 5-methylheptyl group, a 6-methylheptyl group, a 1-ethylhexyl group, a 2-ethylhexyl group, a 3-ethylhexyl group, a 4-ethylhexyl group, a 5-ethylhexyl group, a 1,1-dimethylhexyl group, a 2,2-dimethylhexyl group, a 3,3-dimethylhexyl group, a 4,4-dimethylhexyl group, a 5,5-dimethylhexyl group, a 1-propylpentyl group, a 2-propylpentyl group etc. Those having 1 to 6 carbon atoms, in particular methyl, ethyl, n-propyl and i-propyl, are preferred. Methyl is most preferred.

Examples of alkyl groups which arise by replacement with one or more hetero-analogous groups, such as —O—, —S— or —NH—, are preferably those in which one or more methylene groups are replaced by —O— to form an ether group, such as methoxymethyl, ethoxymethyl, 2-methoxyethylene etc. According to the invention, polyether groups are also included in the definition of alkyl.

Cycloalkyl radicals having 3 to 8 carbon atoms preferably include: a cyclopropyl group, a cyclobutyl group, a cyclopentyl group, a cyclohexyl group, a cycloheptyl group and a cyclooctyl group. A cyclopropyl group, a cyclobutyl group, a cyclopentyl group and a cyclohexyl group are preferred. Heterocyclic alkyl radicals which are formed from cycloalkyl by replacement of methylene by hetero-analogous groups are, for example, 5- or 6-membered heterocyclic radicals, such as tetrahydrofuryl, pyrrolidinyl, morpholinyl, piperidinyl or tetrahydropyranyl, which can optionally be fused with aromatic rings, etc.

In particular, examples of a linear or branched alkyl radical having 1 to 8 carbon atoms and substituted by halogen include:

a fluoromethyl group, a difluoromethyl group, a trifluoromethyl group, a chloromethyl group, a dichloromethyl group, a trichloromethyl group, a bromomethyl group, a dibromomethyl group, a tribromomethyl group, a 1-fluoroethyl group, a 1-chloroethyl group, a 1-bromoethyl group, a 2-fluoroethyl group, a 2-chloroethyl group, a 2-bromoethyl group, a 1,2-difluoroethyl group, a 1,2-dichloroethyl group, a 1,2-dibromoethyl group, a 2,2,2-trifluoroethyl group, a heptafluoroethyl group, a 1-fluoropropyl group, a 1-chloropropyl group, a 1-bromopropyl group, a 2-fluoropropyl group, a 2-chloropropyl group, a 2-bromopropyl group, a 3-fluoropropyl group, a 3-chloropropyl group, a 3-bromopropyl group, a 1,2-difluoropropyl group, a 1,2-dichloropropyl group, a 1,2-dibromopropyl group, a 2,3-difluoropropyl group, a 2,3-dichloropropyl group, a 2,3-dibromopropyl group, a 3,3,3-trifluoropropyl group, a 2,2,3,3,3-pentafluoropropyl group, a 2-fluorobutyl group, a 2-chlorobutyl group, a 2-bromobutyl group, a 4-fluorobutyl group, a 4-chlorobutyl group, a 4-bromobutyl group, a 4,4,4-trifluorobutyl group, a 2,2,3,3,4,4,4-heptafluorobutyl group, a perfluorobutyl group, a 2-fluoropentyl group, a 2-chloropentyl group, a 2-bromopentyl group, a 5-fluoropentyl group, a 5-chloropentyl group, a 5-bromopentyl group, a perfluoropentyl group, a 2-fluorohexyl group, a 2-chlorohexyl group, a 2-bromohexyl group, a 6-fluorohexyl group, a 6-chlorohexyl group, a 6-bromohexyl group, a perfluorohexyl group, a 2-fluoroheptyl group, a 2-chloroheptyl group, a 2-bromoheptyl group, a 7-fluoroheptyl group, a 7-chloroheptyl group, a 7-bromoheptyl group, a perfluoroheptyl group, etc. Fluoroalkyl, difluoroalkyl and trifluoroalkyl are to be mentioned in particular.

Examples of an alkyl radical substituted by hydroxyl include the abovementioned alkyl radicals which contain 1 to 3 hydroxyl radicals, such as, for example, hydroxymethyl, 2-hydroxyethyl, 3-hydroxypropyl etc.

Examples of an alkyl group substituted by cycloalkyl include the abovementioned alkyl radicals which contain 1 to 3, preferably one (optionally substituted) cycloalkyl or heterocyclyl group, such as, for example: cyclohexylmethyl, 2-cyclohexylethyl, 2- or 3-cyclohexylpropyl etc., or such as, for example, morpholinylalkyl, such as 2-morpholinylethyl, morpholinylmethyl etc. Morpholinylmethyl is preferred.

Examples of an alkyl group substituted by aryl or heteroaryl preferably include: straight-chain or branched alkyl having 1 to 8, preferably 1 to 4 carbon atoms, as described above, which is substituted by optionally substituted aryl and/or heteroaryl, as described below. Preferred arylalkyl and/or heteroarylalkyl are benzyl, alkoxybenzyl, such as, in particular, trimethoxybenzyl, pyridylmethyl, furylmethyl, pyrimidylmethyl and pyrrolylmethyl.

Optionally substituted alkoxy includes an optionally substituted alkyl-O group, wherein reference may be made to the above definition with respect to the optionally substituted alkyl group. Preferred alkoxy groups are linear or branched alkoxy groups having up to 6 carbon atoms, such as a methoxy group, an ethoxy group, an n-propyloxy group, an i-propyloxy group, an n-butyloxy group, an i-butyloxy group, a sec-butyloxy group, a t-butyloxy group, an n-pentyloxy group, an i-pentyloxy group, a sec-pentyloxy group, a t-pentyloxy group, a 2-methylbutoxy group, an n-hexyloxy group, an i-hexyloxy group, a t-hexyloxy group, a sec-hexyloxy group, a 2-methylpentyloxy group, a 3-methylpentyloxy group, a 1-ethylbutyloxy group, a 2-ethylbutyloxy group, a 1,1-dimethylbutyloxy group, a 2,2-dimethylbutyloxy group, a 3,3-dimethylbutyloxy group, a 1-ethyl-1-methylpropyloxy group etc. A methoxy group, an ethoxy group, an n-propyloxy group, an i-propyloxy group, an n-butyloxy group, an i-butyloxy group, a sec-butyloxy group, a t-butyloxy group are preferred. The methoxy group is particularly preferred.

Optionally substituted alkenyl in the entire context of the invention preferably includes:

straight-chain or branched-chain alkenyl having 2 to 8 carbon atoms and cycloalkenyl having 3 to 8 carbon atoms, which can optionally be substituted by preferably 1 to 3 identical or different substituents, such as hydroxyl, halogen or alkoxy. Examples include: vinyl, 1-methylvinyl, allyl, 1-butenyl, isopropenyl, cyclopropenyl, cyclobutenyl, cyclopentenyl, cyclohexenyl. Vinyl or allyl are preferred.

With respect to the definition of the optionally substituted alkynyl, reference is made to the above definition of the optionally substituted alkyl, wherein the optionally substituted alkynes include at least one C≡C triple bond. Examples include: ethynyl, propynyl, butynyl, pentynyl and variants thereof optionally substituted as defined above. Ethynyl and optionally substituted ethynyl is preferred.

Optionally substituted aryl in the entire context of the invention preferably includes:

aromatic hydrocarbon radicals having 6 to 14 carbon atoms (the carbon atoms of the possible substituents not being included), which can be mono- or bicyclic and which can be substituted by preferably 1 to 3 identical or different substituents chosen from hydroxyl, halogen, as defined above, cyano, amino, aminocarbonyl, as defined below, mercapto, alkyl, as defined above, acyl, as defined below, and alkoxy, as defined above. Aromatic hydrocarbon radicals having 6 to 14 carbon atoms include, for example: phenyl, naphthyl, phenanthrenyl and anthracenyl, which can optionally be substituted once or several times by identical or different radicals. Phenyl is preferred.

Optionally substituted heteroaryl in the entire context of the invention preferably includes:

heteroaromatic hydrocarbon radicals having 4 to 9 carbon atoms (the carbon atoms of the possible substituents not being included), which contain 1 to 3 identical or different hetero atoms from the series S, O, N and which therefore form 5- to 12-membered heteroaromatic radicals, which can be mono- or bicyclic and which can be substituted by preferably 1 to 3 identical or different substituents chosen, for example, from hydroxyl, halogen, as defined above, cyano, amino, mercapto, alkyl, as defined above, acyl, as defined below, and alkoxy, as defined above.

Heteroaryl includes, for example: pyridyl, pyridyl N-oxide, pyrimidyl, pyridazinyl, pyrazinyl, thienyl, furyl, pyrrolyl, pyrazolyl, imidazolyl, thiazolyl, oxazolyl or isoxazolyl, indolizinyl, indolyl, benzo[b]thienyl, benzo[b]furyl, indazolyl, quinolyl, isoquinolyl, naphthyridinyl, quinazolinyl. 5- or 6-membered aromatic heterocyclyls, such as e.g. pyridyl, pyridyl N-oxide, pyrimidyl, pyridazinyl, furanyl and thienyl, are preferred. Pyridyl, pyrimidyl and furanyl are preferred. Particularly preferred heteroaryl includes: pyridyl, such as pyridin-2-yl, pyridin-3-yl, pyridin-4-yl, pyrimidinyl, such as pyrimidin-2-yl and pyrimidin-5-yl, pyrazin-2-yl, and imidazolyl, such as imidazol-2-yl and imidazol-3-yl, furanyl, such as furan-2-yl and furan-3-yl and thienyl, such as Chien-2-yl and thien-3-yl.

Examples of an aryl group substituted by halogen preferably include: aryl, as described above, which is substituted by 1 to 3 halogen atoms, such as, for example, 2-chloro- or fluorophenyl, 3-chloro- or fluorophenyl, 4-chloro- or fluorophenyl, 2,4-di-(chloro- and/or fluoro)phenyl, 2,5-di-(chloro- and/or fluoro)phenyl, 2,6-di-(chloro- and/or fluoro)phenyl, 3,5-di-(chloro- and/or fluoro)phenyl, 3,6-di-(chloro- and/or fluoro)phenyl, 2,4,6-tri-(chloro- and/or fluoro)phenyl etc. 2-Chlorophenyl, 4-chlorophenyl and 4-fluorophenyl are preferred.

Examples of an aryl or heteroaryl group substituted by alkyl preferably include: aryl and/or heteroaryl, as described above, which is substituted by straight-chain or branched, optionally substituted alkyl having 1 to 8, preferably 1 to 4 carbon atoms, as described above. Preferred alkylaryl and/or alkylheteroaryl are toluoyl, methylpyridyl, methylfuryl, methylpyrimidyl and methylpyrrolyl.

Examples of an aryl or heteroaryl group substituted by substituted alkyl preferably include: aryl and/or heteroaryl, as described above, which is substituted by straight-chain or branched, substituted alkyl having 1 to 8, preferably 1 to 4 carbon atoms, as described above, reference being made to the above definitions with respect to the substitution of the alkyl substituents. A preferred substituted alkyl substituent includes in particular: arylalkyl and/or heteroarylalkyl, such as, in particular, benzyl, alkoxybenzyl, such as, in particular, trimethoxybenzyl, pyridylmethyl, furylmethyl, pyrimidylmethyl and pyrrolylmethyl. Preferred arylalkyl-substituted heteroaryls include: benzylimidazolyl, benzylpyridyl, benzylfuryl, benzylpyrimidyl and methylpyrrolyl, benzylimidazolyl being particularly preferred.

Examples of an aryl and/or heteroaryl group substituted by alkoxy preferably include: aryl or heteroaryl, as described above, which is substituted by 1 to 3 alkoxy radicals, as described above, such as, preferably, 2-methoxyphenyl, 3-methoxyphenyl, 4-methoxyphenyl, 2-ethoxyphenyl, 3-ethoxyphenyl, 4-ethoxyphenyl, 2,4-di-methoxyphenyl etc., and 2-alkoxypyridyl, 3-alkoxypyridyl, 4-alkoxypyridyl, 2-alkoxyfuryl, 3-alkoxyfuryl, 2-alkoxypyrimidyl, 4-alkoxypyrimidyl, 5-alkoxypyrimidyl, 2-alkoxypyrrolyl, 3-alkoxypyrrolyl, 3,5-di-alkoxypyridin-2-yl, 2,5-di-alkoxypyrimidyl, a methoxy group being preferred in particular in these, such as: 2-methoxypyridyl, 3-methoxypyridyl, 4-methoxypyridyl, 2-methoxyfuryl, 3-methoxyfuryl, 2-methoxypyrimidyl, 5-methoxypyrimidyl, 3-methoxypyrrolyl, 3,5-di-methoxypyridin-2-yl, 2,5-Di-methoxypyrimidyl etc. 2-Methoxyphenyl, 3-methoxyphenyl, 4-methoxyphenyl and 2-methoxypyridyl, 3-methoxypyridyl and 4-methoxypyridyl are particularly preferred.

Examples of an aryl and/or heteroaryl group substituted by aminocarbonyl preferably include: aryl or heteroaryl, as described above, which is substituted by 1 to 3 aminocarbonyl radicals, as described below, such as, preferably, benzylamide.

Examples of a cyano-substituted aryl and/or heteroaryl group preferably include: aryl or heteroaryl, as described above, which is substituted by 1 to 3 cyano radicals, such as, preferably, benzonitrile.

Optionally substituted acyl here and in the following includes: optionally substituted aliphatic acyl (alkanoyl=alkyl-CO—, wherein reference may be made to the above definition of optionally substituted alkyl with respect to the alkyl group), optionally substituted aromatic or heteroaromatic acyl (aroyl or hetaroyl=aryl-CO— or heteroaryl-CO—, wherein reference may be made to the above definition of optionally substituted aryl and optionally substituted heteroaryl with respect to aryl and heteroaryl), such as C₁ to C₆ alkanoyl, such as formyl, acetyl, propionyl, butyryl, isobutyryl, valeryl, isovaleryl, pivaloyl, hexanoyl etc., and C₆ to C₁₀ aroyl, such as benzoyl, such as trimethoxy-substituted benzoyl, toluoyl, xyloyl etc., C₆ to C₁₀ heteroaroyl, such as furanoyl, pyridinoyl etc.

Optionally substituted amino in the entire context of the invention preferably includes: amino, mono- or dialkylamino, mono- or diarylamino, alkylarylamino, mono- or diacylamino, wherein reference may be made to the corresponding above definition for optionally substituted alkyl, optionally substituted aryl and optionally substituted acyl with respect to alkyl, aryl and acyl. Mono- or dialkylamino in this context includes in particular: straight-chain or branched mono- or dialkylamino having 1 to 8, preferably 1 to 4 saturated or unsaturated carbon atoms, optionally substituted as described above, in each alkyl group, in particular methylamine, dimethylamino.

Optionally substituted aminocarbonyl in the context of the entire invention preferably represents carbamoyl (H₂NCO—) or mono- or dialkylaminocarbonyl (H(alkyl)N—CO— or (alkyl)₂N—CO—), wherein reference may be made to the above explanations for optionally substituted alkyl with respect to the definition of alkyl.

Furthermore, optionally substituted aminosulfonyl in the context of the entire invention represents in particular sulfamoyl (H₂N—SO₂—) or mono- or dialkylaminosulfonyl (alkyl)₂N—SO₂, wherein reference may be made to the above explanations for optionally substituted alkyl with respect to the definition of alkyl.

Optionally substituted alkoxycarbonyl (RO(O═)C—) includes the abovementioned optionally substituted alkoxy with respect to the definition of alkoxy, and methoxycarbonyl and ethoxycarbonyl are preferred.

In the general formula (I), the style of writing for the substituent(s) R¹:

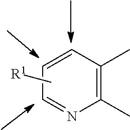

means that R¹ denotes the three substituent positions (2, 3 and 4) of the quinoline skeleton identified with the arrows. In this context, R¹ can be hydrogen, which means that the quinoline is not substituted at the positions mentioned, or R¹ in the context of the definitions given in claim 1 can include one, two or three identical or different substitutions on the positions mentioned.

### PREFERRED EMBODIMENTS

In a preferred embodiment, the compound of the formula (I) has the following substituent definitions:

R¹, R² and R⁷ are identical or different and are each chosen from the group consisting of:
hydrogen,
hydroxyl,
halogen,
optionally substituted alkyl,
optionally substituted alkoxy,
optionally substituted aryl,
optionally substituted heteroaryl;
   R⁶ is chosen from the group consisting of:
hydrogen,
optionally substituted alkyl,
optionally substituted aryl,
optionally substituted heteroaryl;
   R³ is chosen from the group consisting of:
hydrogen,
optionally substituted alkyl,
optionally substituted aryl,
optionally substituted heteroaryl;
optionally substituted acyl; and
   R⁴ and R⁵ are identical or different and are each chosen from the group consisting of:
hydrogen,
optionally substituted alkyl,
optionally substituted aryl,
optionally substituted heteroaryl,
   or R⁴ and R⁵ together with the nitrogen atom to which they are bonded form a saturated or unsaturated, optionally substituted 5- to 8-membered ring which can optionally contain further hetero atoms.

In a further more preferred embodiment, the compound of the formula (I) has the following substituent definitions:

R¹, R² and R⁷ are identical or different and are each chosen from the group consisting of:
hydrogen,
hydroxyl,
halogen,
optionally substituted alkyl,
optionally substituted alkoxy;
   R⁶ is chosen from the group consisting of:
hydrogen,
optionally substituted alkyl,
optionally substituted aryl,
optionally substituted heteroaryl;
   R³ is chosen from the group consisting of:
hydrogen,
optionally substituted alkyl,
optionally substituted acyl; and
   R⁴ and R⁵ are identical or different and are each chosen from the group consisting of:
hydrogen,
optionally substituted aryl,
optionally substituted heteroaryl,
   or R⁴ and R⁵ together with the nitrogen atom to which they are bonded form a saturated or unsaturated, optionally substituted 5- to 6-membered ring which can optionally contain further hetero atoms.

In a further more preferred embodiment, the compound of the formula (I) has the following substituent definitions:

### R¹:

hydrogen;

### R²:

hydrogen or
halogen;

### R³:

hydrogen, optionally substituted acyl, in particular optionally substituted aroyl or optionally substituted heteroaroyl;
   R⁴ and R⁵ are identical or different and denote:
hydrogen,
optionally substituted aryl,
optionally substituted heteroaryl, or
R⁴ and R⁵ together with the nitrogen atom to which they are bonded form a saturated or unsaturated, optionally substituted 6-membered ring which can contain further hetero atoms.

### R⁶:

hydrogen,
optionally substituted aryl, or
optionally substituted heteroaryl; and

### R⁷:

hydrogen.

In a further more preferred embodiment, the compound of the formula (I) has the following substituent definitions:

R¹ is hydrogen,
R² is chosen from
hydrogen or
chlorine
   R³ is chosen from
hydrogen, optionally substituted furoyl or optionally substituted benzoyl;
   R⁴ and R⁵ are identical or different and denote:
hydrogen,
optionally substituted heteroaryl, or
R⁴ and R⁵ together with the nitrogen atom to which they are bonded form a saturated, optionally substituted 6-membered ring which contains a further hetero atom;
   R⁶ denotes:
hydrogen,
optionally halogen-substituted phenyl or
pyridinyl; and
   R⁷ is hydrogen.

In a further preferred embodiment, at least one of the substituents R², R³, R⁴, R⁵, R⁶ and R⁷ of the compound of the formula (I) has the definition as in the in the last two abovementioned further more preferred embodiments.

A further more preferred embodiment relates to compounds of the formula (I) with the following substituent definitions:

R¹, R² and R⁷ are identical or different and are each chosen from the group consisting of:
hydrogen,
hydroxyl,
halogen,
optionally substituted alkyl,
optionally substituted alkoxy;
   R⁶ is chosen from the group consisting of:
hydrogen,
optionally substituted alkyl,
optionally substituted aryl,
optionally substituted heteroaryl;
   R³ is chosen from the group consisting of:
hydrogen,
optionally substituted alkyl,
optionally substituted acyl; and
   R⁴ and R⁵ are identical or different and are each chosen from the group consisting of:
hydrogen,
optionally substituted alkyl,
optionally substituted aryl,
optionally substituted heteroaryl;
   or R⁴ and R⁵ together with the nitrogen atom to which they are bonded form a saturated or unsaturated, optionally substituted 5- to 6-membered ring which can optionally contain further hetero atoms.

In a further more preferred embodiment, the compound of the formula (I) has the following substituent definitions:

### R¹:

hydrogen;

### R²:

hydrogen,
halogen or
optionally substituted alkyl, in particular cycloalkyl-substituted alkyl;

### R³:

hydrogen,
optionally substituted alkyl, in particular optionally substituted arylalkyl or optionally substituted heteroarylalkyl, or
optionally substituted acyl, in particular optionally substituted aroyl or optionally substituted heteroaroyl;
   R⁴ and R⁵ are identical or different and denote:
hydrogen,
optionally substituted alkyl,
optionally substituted heteroaryl, or
R⁴ and R⁵ together with the nitrogen atom to which they are bonded form a saturated or unsaturated, optionally substituted 6-membered ring which can contain further hetero atoms.

### R⁶:

hydrogen,
optionally substituted aryl, or
optionally substituted heteroaryl; and

### R⁷:

hydrogen.

In a further more preferred embodiment, the compound of the formula (I) has the following substituent definitions:

R¹ is hydrogen,
R² is chosen from
hydrogen,
chlorine or
morpholinylalkyl, such as morpholinylmethyl;
   R³ is chosen from
hydrogen,
optionally substituted benzyl or
optionally substituted furoyl or optionally substituted benzoyl;
   R⁴ and R⁵ are identical or different and denote:
hydrogen,
optionally substituted alkyl,
optionally substituted heteroaryl, or
R⁴ and R⁵ together with the nitrogen atom to which they are bonded form a saturated, optionally substituted 6-membered ring which contains one or no further hetero atom;
   R⁶ denotes:
hydrogen,
optionally halogen-, alkyl-, alkoxy-, aminocarbonyl- or cyano-substituted phenyl or
optionally substituted pyridinyl, pyrazinyl, imidazolyl or thienyl; and
   R⁷ is hydrogen.

In preferred embodiments of the general formula (I), the individual substituents each have the following definitions:

R¹ is preferably hydrogen (at all three positions, as explained above).

R² is hydrogen or halogen, preferably chlorine.

R³ is hydrogen or optionally substituted acyl, in particular optionally substituted aroyl or optionally substituted heteroaroyl; preferably (optionally substituted) furoyl or optionally substituted benzoyl; preferably alkoxy-substituted benzoyl, more preferably methoxy-substituted benzoyl, such as trimethoxybenzoyl.

R⁴ and R⁵ are identical or different and denote:
hydrogen,
optionally substituted aryl,
optionally substituted heteroaryl, preferably pyridinyl, such as pyridin-2-yl or pyridin-3-yl or
R⁴ and R⁵ together with the nitrogen atom to which they are bonded form a saturated or unsaturated, optionally substituted 6-membered ring which can contain further hetero atoms chosen from O, N, S, preferably O, preferably morpholino.

R⁶ is hydrogen, optionally substituted aryl, such as halogen-substituted phenyl, such as chlorophenyl, such as 4-chlorophenyl, or optionally substituted heteroaryl, such as pyridin-2-yl or pyridin-3-yl.

R⁷ is hydrogen.

In more preferred embodiments of the general formula (I), the individual substituents each have the following definitions:

R¹ is preferably hydrogen (at all three positions, as explained above).

R² is hydrogen or halogen, such as preferably chlorine, or morpholinylalkyl, such as preferably morpholinylmethyl.

R³ is hydrogen or optionally substituted alkyl, in particular optionally substituted arylalkyl or heteroarylalkyl; preferably (optionally substituted) benzyl, preferably alkoxy-substituted benzyl, more preferably methoxy-substituted benzyl, such as trimethoxybenzyl, or R³ is optionally substituted acyl, in particular optionally substituted aroyl or optionally substituted heteroaroyl; preferably (optionally substituted) furoyl or optionally substituted benzoyl; preferably alkoxy-substituted benzoyl, more preferably methoxy-substituted benzoyl, such as trimethoxybenzoyl.

R⁴ and R⁵ are identical or different and denote:
hydrogen,
optionally substituted alkyl,
optionally substituted aryl,
optionally substituted heteroaryl, preferably pyridinyl, such as pyridin-2-yl or pyridin-3-yl or
R⁴ and R⁵ together with the nitrogen atom to which they are bonded form a saturated or unsaturated, optionally substituted 6-membered ring which can contain further hetero atoms chosen from O, N, S, preferably O, preferably morpholino, piperidine or piperazine optionally substituted once or several times by identical or different substituents, wherein reference may be made to the above substituent definitions with respect to the possible substituents of the heterocyclyls formed by R⁴ and R⁵, from which (optionally substituted) alkyl, alkoxycarbonyl, aryl and/or heteroaryl substituents are particularly preferred. Preferred alkyl substituents are, in particular, one or more methyl or ethyl groups, hydroxyalkyl, such as, in particular, hydroxyethyl, arylalkyl, such as, in particular benzyl; preferred alkoxycarbonyl substituents are methoxycarbonyl or ethoxycarbonyl; preferred aryl substituents are optionally halogen-substituted phenyl, preferably chloro- or fluorophenyl; preferred heteroaryl substituents are pyridyl or pyridinyl.

In a particularly preferred embodiment, R⁴ and R⁵ together with the nitrogen atom to which they are bonded form morpholino, 2,4-dimethylmorpholino, piperidine, benzylpiperidine, fluorophenyl-piperidine, N-methylpiperazine, N-hydroxyethylpiperazine, N-benzylpiperazine, N-ethoxycarbonylpiperazine or N-pyridinyl-piperazine.

R⁶ is hydrogen, optionally substituted aryl, such as halogen-substituted phenyl, such as fluorophenyl, such as 4-fluorophenyl, or chlorophenyl, such as 2-chlorophenyl or 4-chlorophenyl, alkyl-substituted phenyl, such as toluoyl, such as 2-toluoyl or 4-toluoyl, alkoxy-substituted phenyl, such as methoxyphenyl, such as 2-methoxyphenyl or 4-methoxyphenyl, aminocarbonyl-substituted phenyl, such as 4-benzamide, or cyano-substituted phenyl, such as 4-benzonitrile, or optionally substituted heteroaryl, such as (optionally substituted) pyridin-2-yl or pyridin-3-yl or pyridin-4-yl, such as alkoxy-substituted pyridinyl, such as methoxypyridinyl, such as 6-methoxypyridin-3-yl, or (optionally substituted) pyrazinyl or optionally substituted imidazolyl, such as imidzol-2-yl or imidazol-3-yl, such as alkyl-substituted imidazolyl, such as arylalkyl-substituted imidazolyl, such as 1-benzyl-imidazol-2-yl, or (optionally substituted) thienyl, such as thien-2-yl.

R⁷ is hydrogen.

Particularly preferred compounds of the general formula (I) are shown in the following table:

| | |
|---|---|
| | (I) |
| 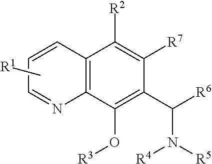 | |

| Example | Compound | R¹ | R² | R³ | R⁴ | R⁵ | R⁶ | R⁷ |
|---|---|---|---|---|---|---|---|---|
| 1 | 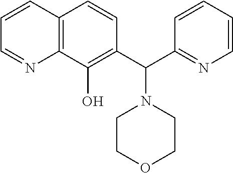 | H | H | H | 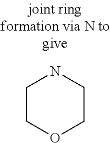 | | 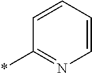 | H |
| 2 | 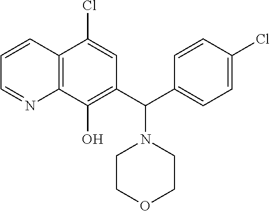 | H | Cl | H | 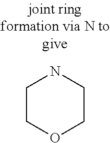 | | 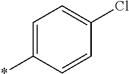 | H |
| 3 | 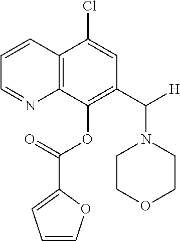 | H | Cl | 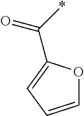 | 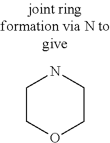 | | H | H |
| 4 | 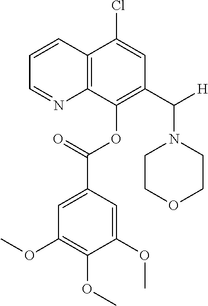 | H | Cl | 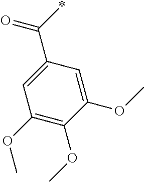 | 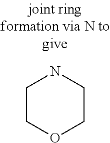 | | H | H |
| 5 | 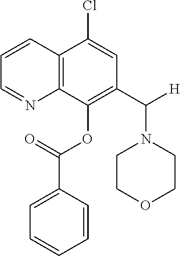 | H | Cl | 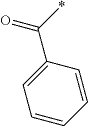 | 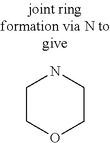 | | H | H |
| 6 | 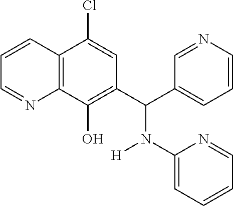 | H | Cl | H | H | 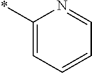 | 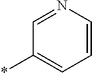 | H |
| 7 | 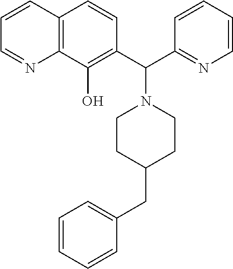 | H | H | H | 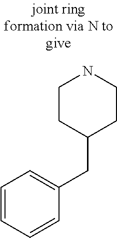 | | 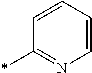 | H |
| 8 | 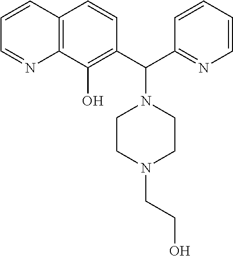 | H | H | H | 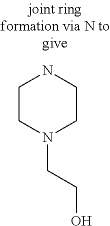 | | 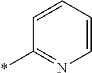 | H |
| 9 | 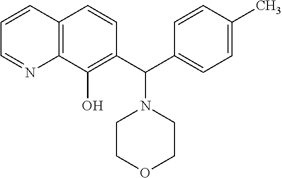 | H | H | H | 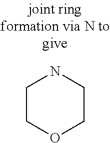 | | 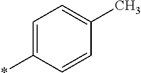 | H |
| 10 | 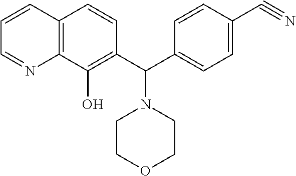 | H | H | H | 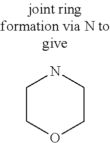 | | 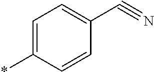 | H |
| 11 | 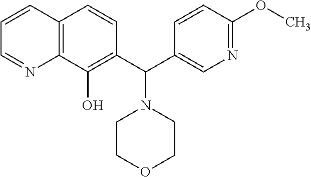 | H | H | H | 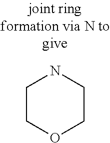 | | 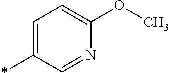 | H |
| 12 | 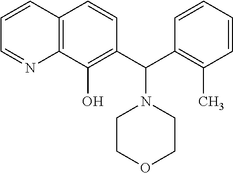 | H | H | H | 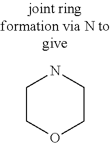 | | 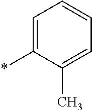 | H |
| 13 | 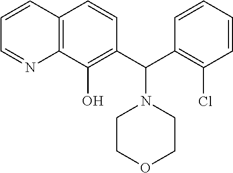 | H | H | H | 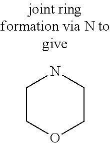 | | 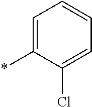 | H |
| 14 | 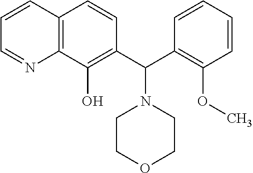 | H | H | H | 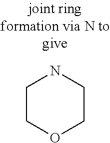 | | 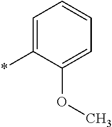 | H |
| 15 | 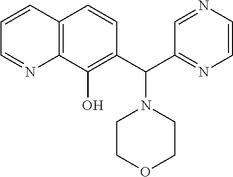 | H | H | H | 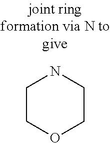 | | 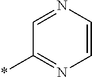 | H |
| 16 | 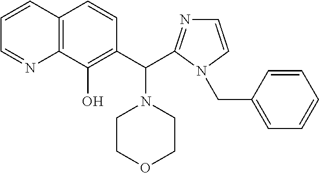 | H | H | H | 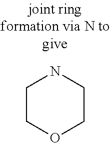 | | 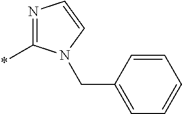 | H |
| 17 | 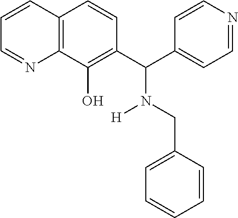 | H | H | H | H | 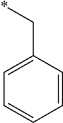 | 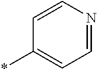 | H |
| 18 | 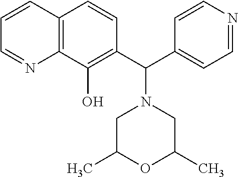 | H | H | H | 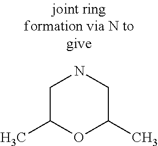 | | 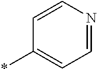 | H |
| 19 | 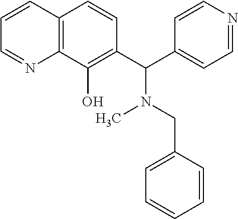 | H | H | H | CH₃ | 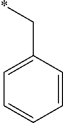 | 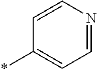 | H |

| | | | | | | | |
|---|---|---|---|---|---|---|---|
| 20 | 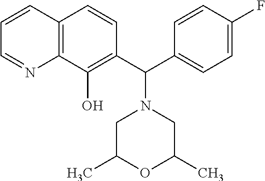 | H | H | H | 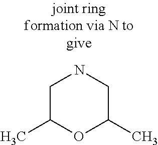 | 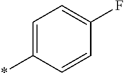 | H |
| 21 | 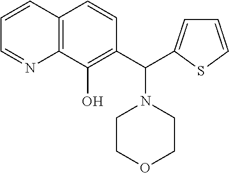 | H | H | H | 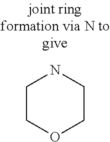 | 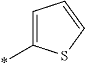 | H |
| 22 | 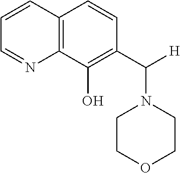 | H | H | H | 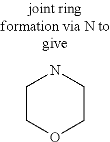 | H | H |
| 23 | 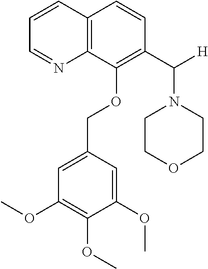 | H | H | 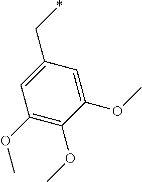 | 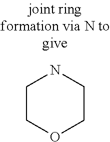 | H | H |
| 24 | 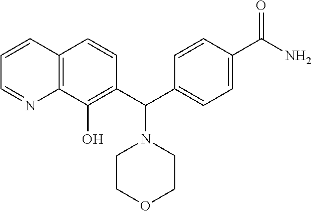 | H | H | H | 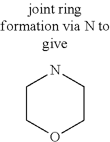 | 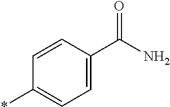 | H |
| 25 | 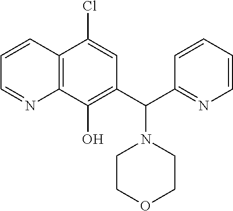 | H | Cl | H | 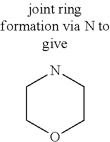 | 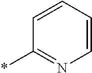 | H |
| 26 | 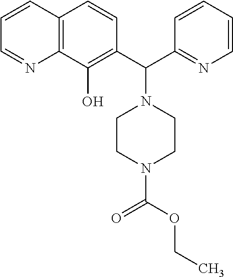 | H | H | H | 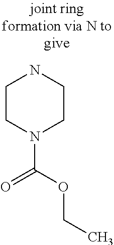 | 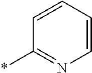 | H |
| 27 | 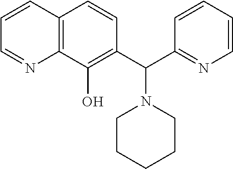 | H | H | H | 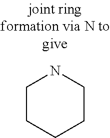 | 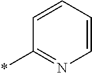 | H |
| 28 | 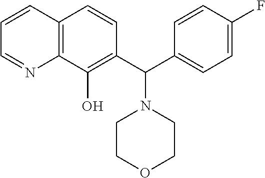 | H | H | H | 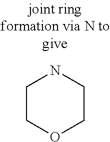 | 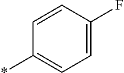 | H |
| 29 | 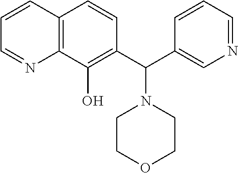 | H | H | H | 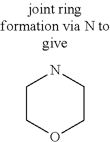 | 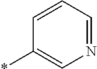 | H |
| 30 | 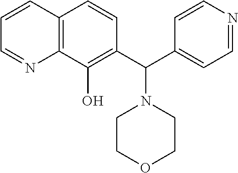 | H | H | H | 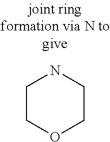 | 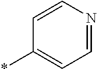 | H |
| 31 | 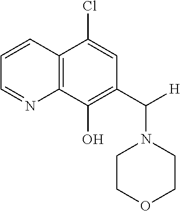 | H | Cl | H | 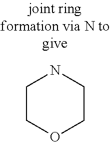 | H | H |
| 32 | 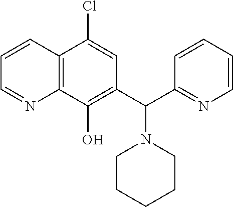 | H | Cl | H | 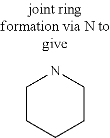 | 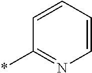 | H |
| 33 | 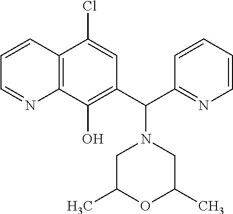 | H | Cl | H | 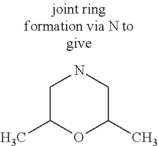 | 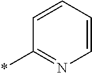 | H |
| 34 | 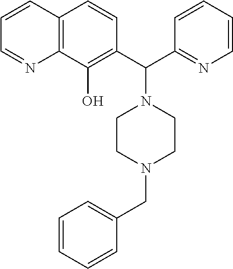 | H | H | H | 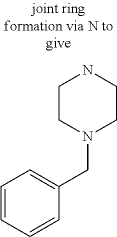 | 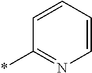 | H |
| 35 | 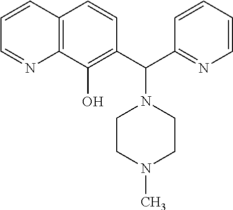 | H | H | H | 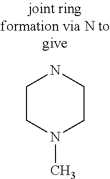 | 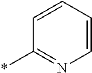 | H |
| 36 | 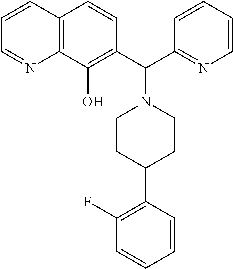 | H | H | H | 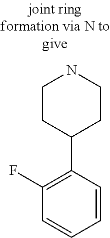 | 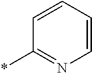 | H |
| 37 | 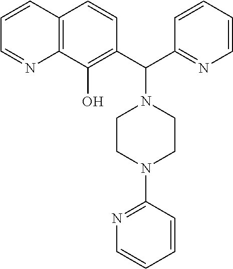 | H | H | H | 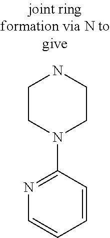 | 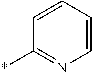 | H |
| 38 | 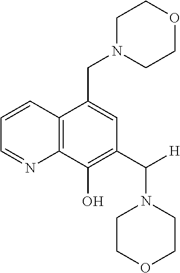 | H | 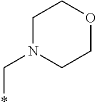 | H | 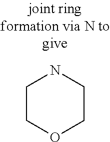 | H | H |
| 39 | 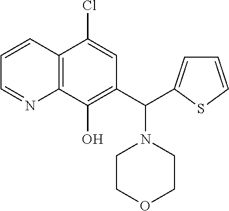 | H | Cl | H | 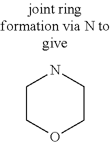 | 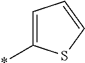 | H |
| 40 | 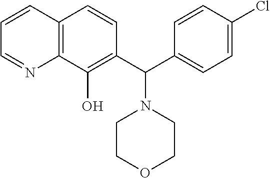 | H | H | H | 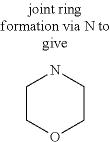 | 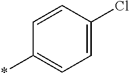 | H |
| 41 | 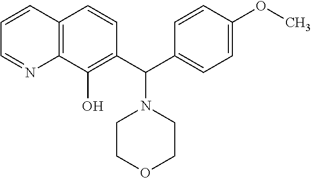 | H | H | H | 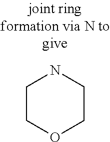 | 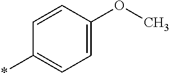 | H |

and pharmaceutically acceptable salts thereof.

In particular, the present invention also relates to novel compounds of the general formula (I) with the meaning of the substituents as described above, the following compounds being excluded.

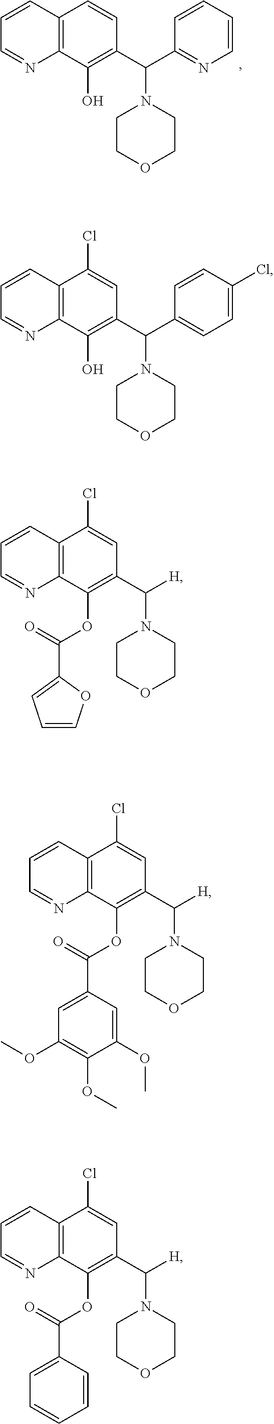

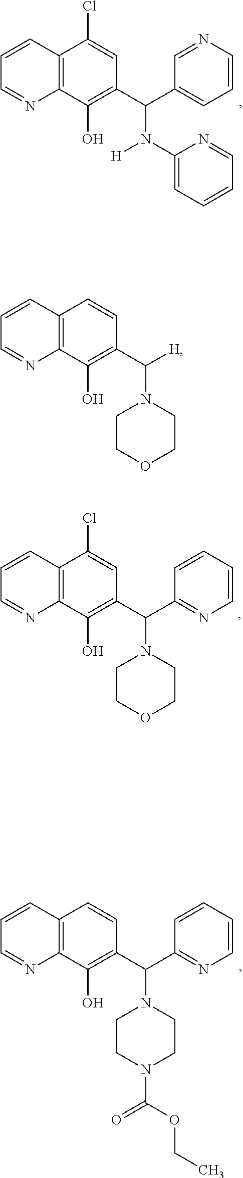

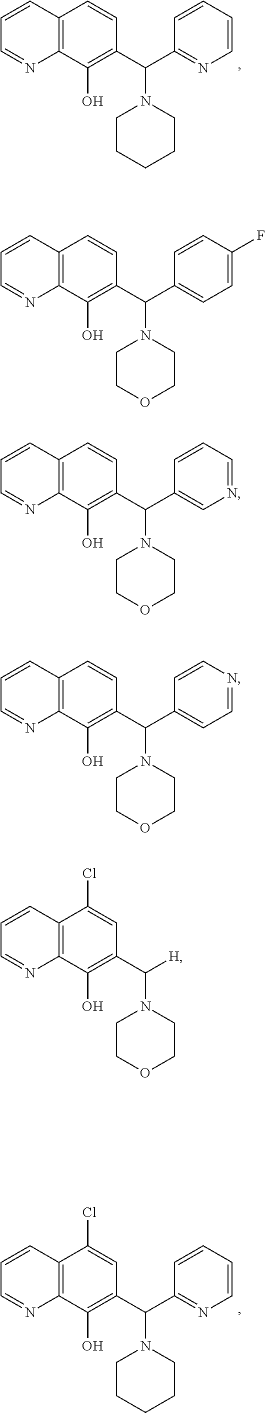

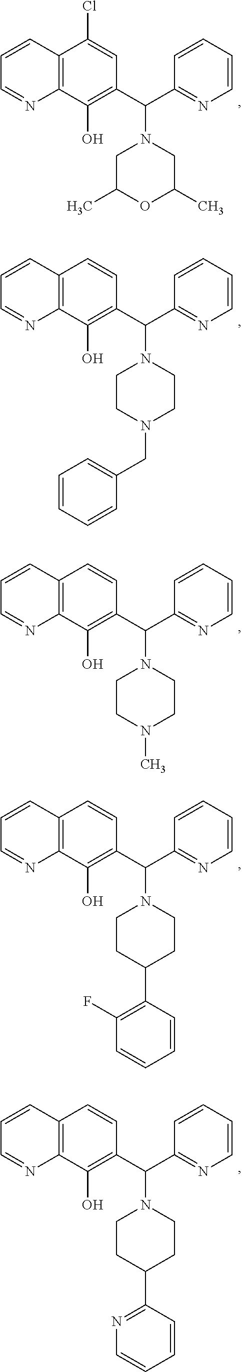

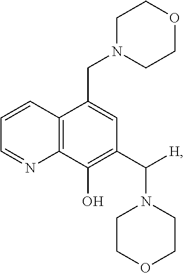

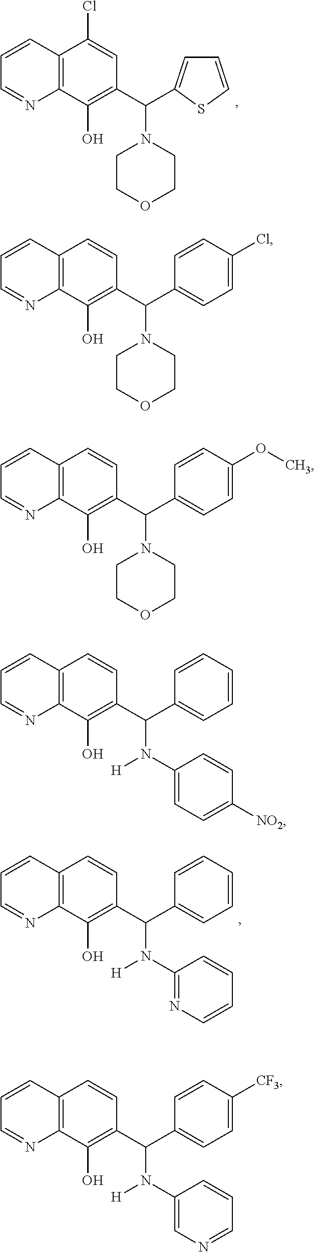

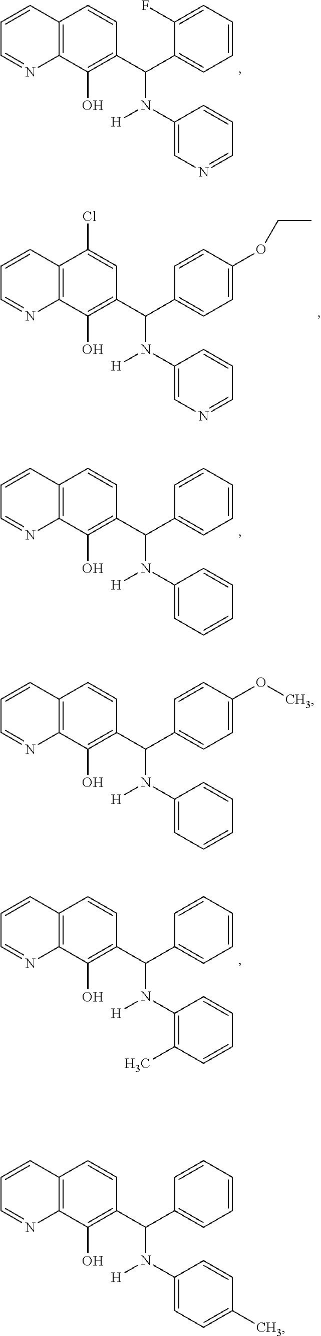

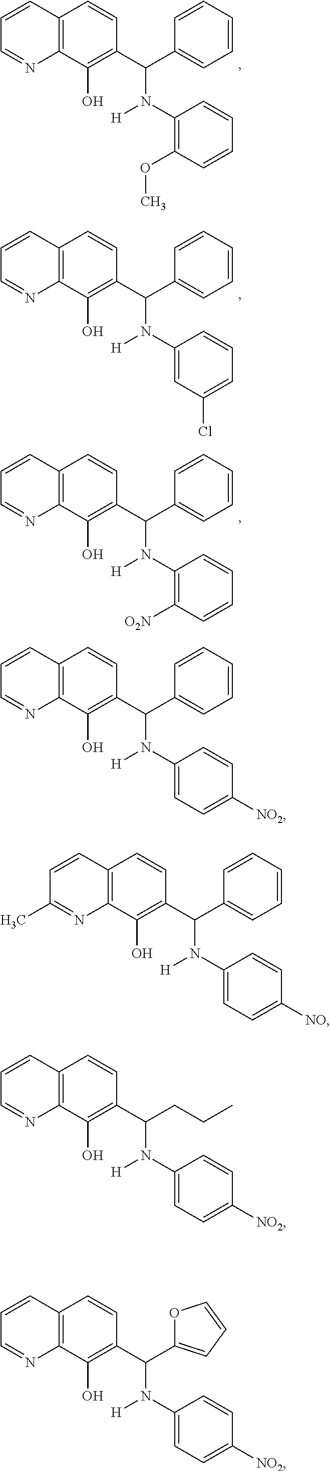

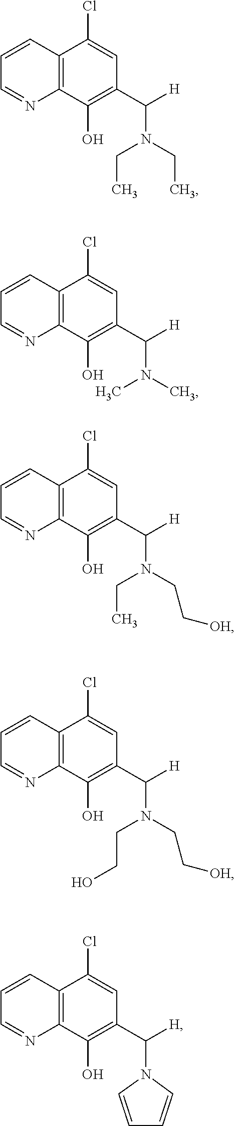

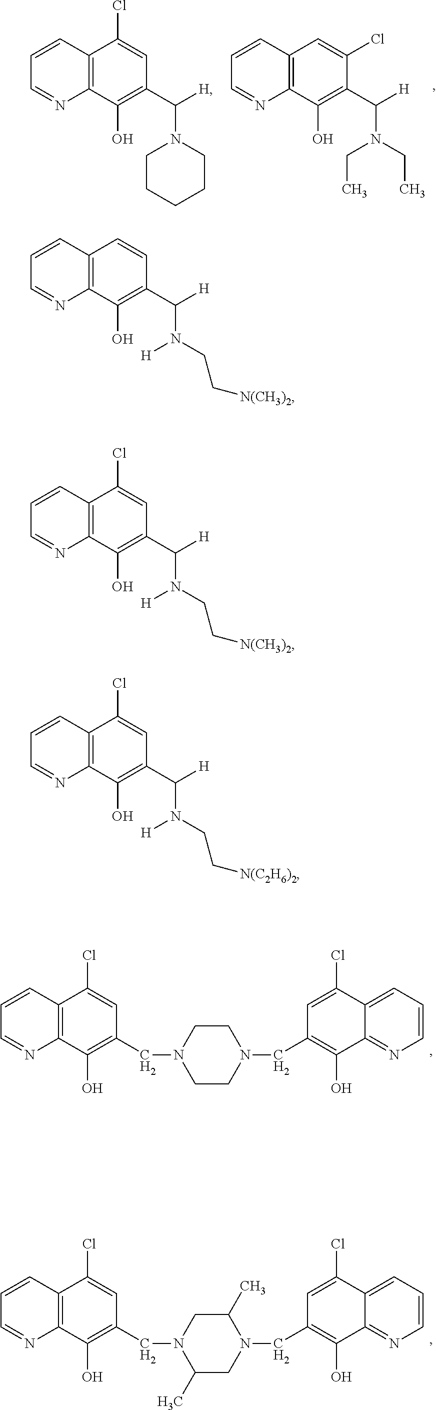

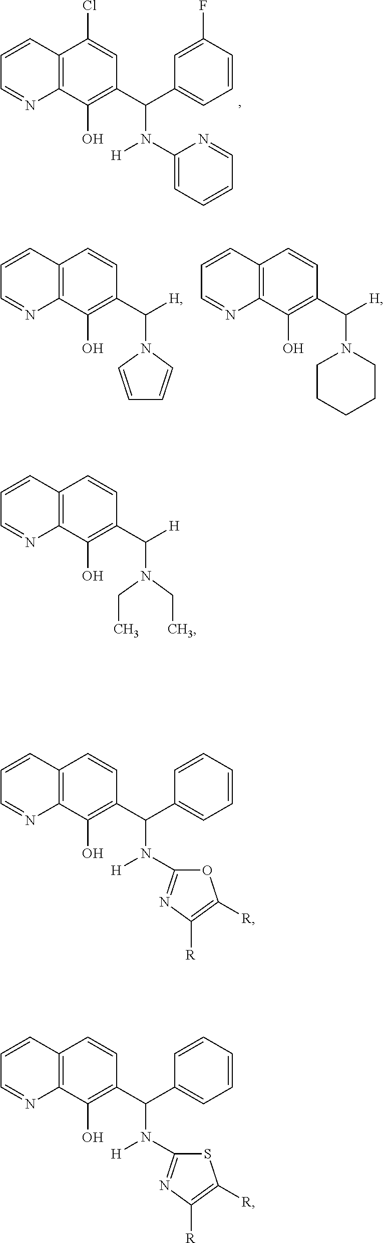

R=optionally substituted R=optionally substituted

In principle, in the context of the present invention it is possible to combine the individual preferred, more preferred or particularly preferred meanings for the substituents R¹ to R⁷ with one another. That is to say that the present invention includes compounds of the general formula (I) in which, for example, the substituent R⁶ and/or the substituents R⁴ and R⁵ have a preferred or more preferred meaning and the substituents R¹, R² and/or R³ have the general meaning or the substituent R⁶ and/or the substituents R⁴ and R⁵ have a general meaning and the substituents R¹, R² and/or R³ have a preferred or more preferred meaning etc.

Depending on their structure, if asymmetric carbon atoms are present the compounds according to the invention can exist in stereoisomeric forms (enantiomers, diastereomers). The invention therefore includes the use of the enantiomers or diastereomers and their particular mixtures. The enantiomerically pure forms can optionally be obtained by conventional processes of optical resolution, such as by fractional crystallization of diastereomers therefrom by reaction with optically active compounds. If the compounds according to the invention can occur in tautomeric forms, the present invention includes the use of all the tautomeric forms.

An asymmetric carbon atom can be present, for example, at the marked position:

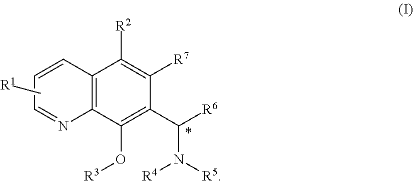

The compounds provided according to the invention can be present as mixtures of various possible isomeric forms, in particular of stereoisomers, such as e.g. E, Z, syn and anti, and optical isomers. Both the E and the Z isomers and the optical isomers, and any desired mixtures of these isomers are claimed.

The compounds according to the invention of the general structural formula (I) can in principle be obtained by the processes explained in the following.

The starting substance for the synthesis of compounds of the general formula (I) wherein R¹ is hydrogen is the commercially obtainable 8-hydroxyquinoline (II). In this, (II) is reacted with a suitable aldehyde of the general formula (III) and an amine of the general formula (IV) in a Mannich reaction under reaction conditions familiar to the person skilled in the art [Phillips, JACS, 75, 1953, 3768; Tripathy, JICSAH, 35, 1958, 407-409; Banerjee, JICSAH, 66, 1989, 319-321], e.g. in the sense of an aminoalkylation to give a product of the general formula (I′) Product (I′) can then optionally be derivatized further by methods familiar to the person skilled in the art to give compounds of the general formula (I) wherein R¹ is hydrogen. The reaction with R³—X can take place therein, for example, in the presence of sodium hydride (NaH).

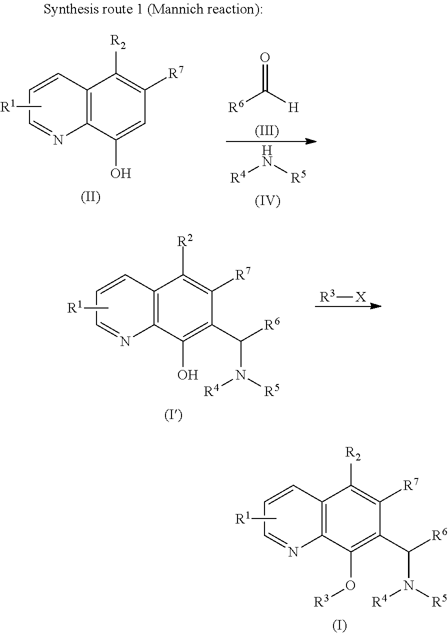

where R¹, R² and R⁷=H

In the synthesis route shown by way of example here, the substituents R² and R⁷ of the general formula (I) are likewise hydrogen. For the preparation of corresponding compounds of the formula (I) wherein R² and R⁷ are substituents which differ from hydrogen, either 8-hydroxyquinolines (II) which are already correspondingly substituted on R² and/or R⁷ are employed as starting compounds, or the compounds obtainable by the synthesis route described are then reacted further by suitable methods generally known to the person skilled in the art to give the correspondingly R²- and/or R⁷-substituted compounds (I).

In order to obtain compounds of the formula (I) according to the invention in which R¹ is a substituent which differs from hydrogen, various preparation processes are available, depending on the desired position of the R¹ substituent.

Thus, compounds of the general structural formula (I) wherein R¹ is a substituent in the ortho position to the nitrogen atom of the quinoline base skeleton are represented by the formula (Ia):

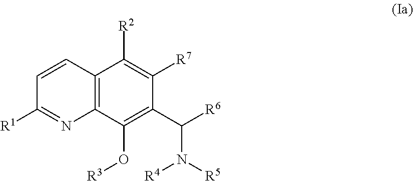

Compounds (Ia) substituted in this way by R¹ (where R¹≠H) are accessible, for example, by combination of a Doebner-v. Miller quinaldine synthesis and the Mannich from synthesis route 1 shown above, the Doebner-v. Miller quinaldine synthesis preceding the Mannich reaction and delivering the starting substances for the Mannich reaction.

In the Doebner-v. Miller quinaldine synthesis, commercially available 2-hydroxyaniline (VI) is reacted as the starting substance with α,β-unsaturated aldehydes of the general formula (VII) under reaction conditions familiar to the person skilled in the art to give the corresponding hydroxyquinolines of the general formula (VIIIa).

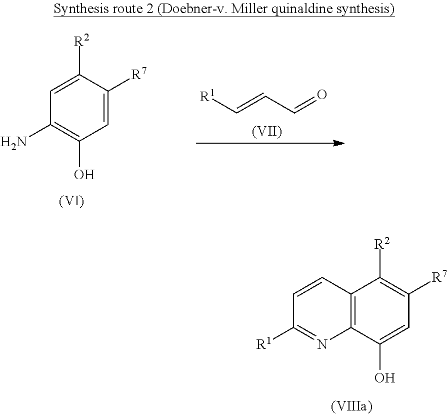

where R² and R⁷=H

Substances of the general formula (VIIIa) can then be converted into the products of the general formula (Ia) analogously to the process described under synthesis route I by means of a Mannich reaction by reaction with products of the formulae (III) and (IV) to give the correspondingly substituted compounds (Va) and optionally subsequent derivatization with an R³ substituent.

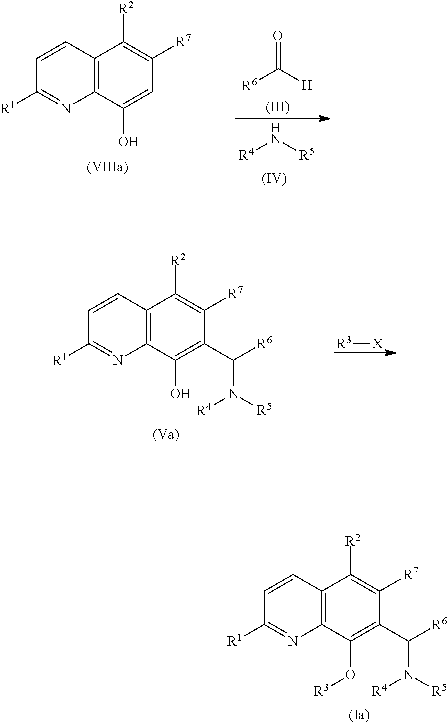

where R² and R⁷=H and R¹≠H

Compounds of the general structural formula (I) wherein R¹ is a substituent in the meta or para position instead of substitution in the ortho position to the quinoline nitrogen are represented by the formulae (Ib) and (Ic).

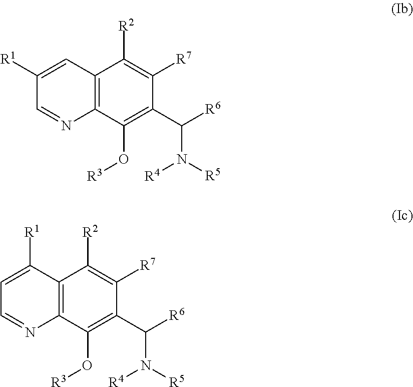

These can be prepared either likewise by means of suitable substituted aldehydes of the general formula (VII) via the Doebner-v. Miller quinaldine synthesis with a subsequent Mannich reaction, or in suitable cases also by combination of the Mannich reaction from synthesis route 1 shown above with a Friedländer synthesis, the Friedländer synthesis preceding the Mannich reaction and delivering the starting substances for the Mannich reaction [M. Phillips, JACS, 70, 1947, 410];

The Friedländer synthesis is based on an alkaline condensation of o-aminobenzaldehyde with the general structural formula (X) with aldehydes or ketones (XI) which have an active CH₂ group in the adjacent position to the keto group [C. C. Cheng, Org Rxs, 28, 1982, 37], as shown in the following reaction equation. The Friedländer synthesis can also be carried out under acid catalysis. This is often a better route to the desired product precisely in sterically bulkier systems.

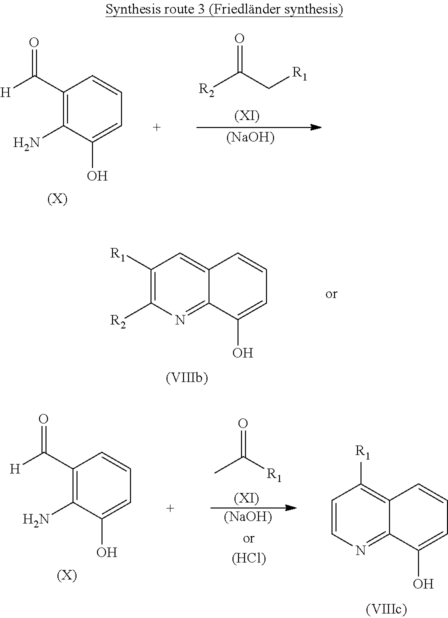

The substituted quinoline base skeletons obtainable by this means can be subsequently converted into the compounds of the general structural formulae (I) or (Ia), (Ib) and (Ic) by suitable substitution reactions generally known to the person skilled in the art.

In the context of the invention, compounds R—X, such as, in particular, R³—X, but optionally also R¹—X, R²—X or R⁷—X, and R⁸—C(═O)—X, as defined below, are those wherein R¹, R², R³ and R⁷ have the meanings as defined above, and wherein X is a usual leaving group, such as, for example, halogen.

In order to obtain compounds of the general formula (I) in which R³ represents an acyl group, the compounds of the formula (I) obtainable by the processes described above can be converted into compounds of the general formula (I″) in accordance with the following reaction equation:

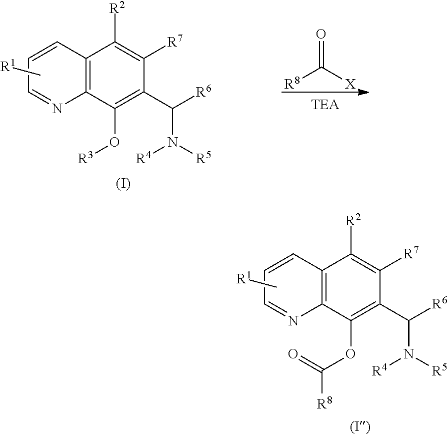

In particular, processes according to the synthesis route described in detail in the following, wherein the meaning of the substituents R¹ to R⁸ corresponds to the above definitions and wherein the abbreviations used have the meaning as defined in particular in the following preparation examples, are preferred.

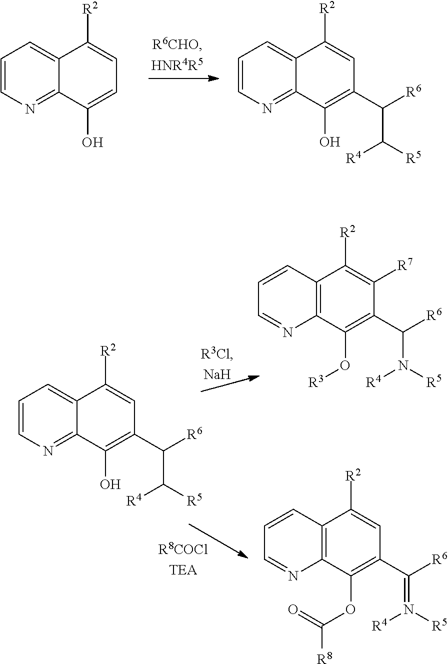

In the context of the invention, in the compounds R⁸ is substituents of the optionally substituted acyl group R³ according to the invention which are suitable and preferred according to the invention, as defined in the context of the present invention. R⁸ thus has the meanings as defined above, in particular R⁸ preferably has the meaning of (optionally substituted) alkyl, aryl or heteroaryl.

The reaction paths shown here are reaction types which are known per se and which can be carried out in a manner known per se. By reaction with a pharmaceutical acceptable base or acid, corresponding salts are obtained.

The reaction of the various reaction partners can be carried out in various solvents, and in this respect is not subject to a particular limitation. Corresponding examples of suitable solvents are thus water, dichloroethane, methylene chloride, dimethoxyethane, diglyme, acetonitrile, butyronitrile, THF, dioxane, ethyl acetate, butyl acetate, dimethylacetamide, toluene and chlorobenzene. It is moreover possible to carry out the reaction in an essentially homogeneous mixture of water and solvents if the organic solvent is miscible with water.

The reaction according to the invention of the reaction partners is carried out, for example, at room temperature. However, temperatures above room temperature, for example up to 50° C., and temperatures below room temperature, for example down to −20° C. or less, can also be used.

The pH at which the reaction according to the invention of the reaction partners is carried out in the Doebner-v. Miller quinaldine synthesis is suitably adjusted.

The pH in the Doebner-v. Miller quinaldine synthesis is preferably adjusted by addition of an acid. In principle both organic and inorganic acids can be used as acids. Preferably, inorganic acids, such as, for example, HCl, HBr, HF, H₂SO₄, H₃PO₄, or organic acids, such as CF₃COOH, CH₃COOH, p-toluenesulfonic acid are used. Particularly preferably, inorganic acids, very particularly preferably HCl and H₂SO₄, are used.

The pH in the Friedländer synthesis is expediently adjusted, preferably by addition of a base. In principle both organic and inorganic bases can be used as bases. Preferably, inorganic bases, such as, for example, LiOH, NaOH, KOH, Ca(OH)₂, Ba(OH)₂, Li₂CO₃, K₂CO₃, Na₂CO₃, NaHCO₃, NaH, or organic bases, such as amines (such as, for example, preferably triethylamine (TEA), diethylisopropylamine), Bu₄NOH, piperidine, morpholine, alkylpyridines, are used. Particularly preferably, inorganic bases, very particularly preferably LiOH, NaOH and KOH, are used.

If the Friedländer synthesis is carried out under acid catalysis, the pH is preferably adjusted by addition of an acid, such as, preferably, those such as are mentioned above for the Doebner-v. Miller quinaldine synthesis.

A person skilled in the art is in a position here to choose the most suitable solvent and the optimum reaction conditions, in particular with respect to temperature, pH and solvent, for the corresponding synthesis route.

Preferably, after the reaction of the reaction partners described above, the end products according to the invention is obtained from the resulting crude product by means of preparative HPLC under neutral conditions and/or by means of column chromatography.

The inventors have found, surprisingly, that the compounds provided by the present invention and represented by the general structural formula (I) show an action as a hepcidin antagonist and are therefore suitable for use as medicaments for treatment of hepcidin-mediated diseases and the symptoms accompanied by or associated with these. In particular, the compounds according to the invention are suitable in use for treatment of disorders in iron metabolism, in particular for treatment of iron deficiency diseases and/or anaemias, in particular ACD and AI.

The medicaments containing the compounds of the general structural formula (I) are suitable in this context for use in human and veterinary medicine.

The present invention thus also provides the compounds of the general structural formula (I) according to the invention for use as medicaments.

Compounds of the general structural formula (I) are preferred for use as medicaments, the following compounds being excluded:

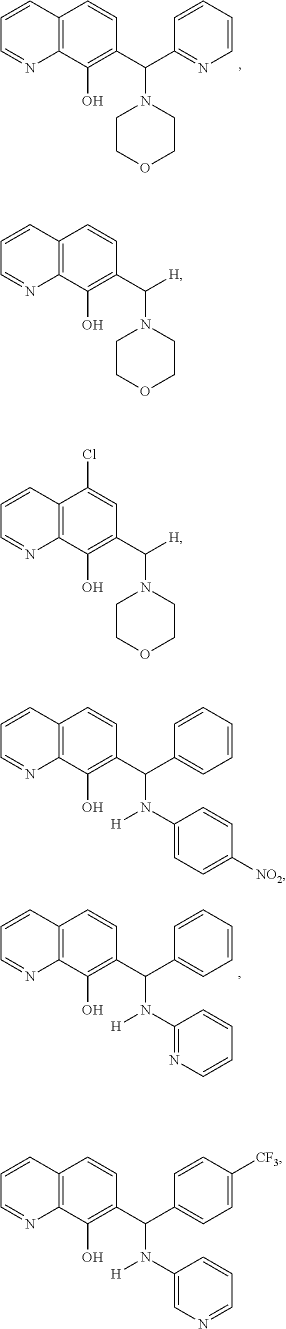

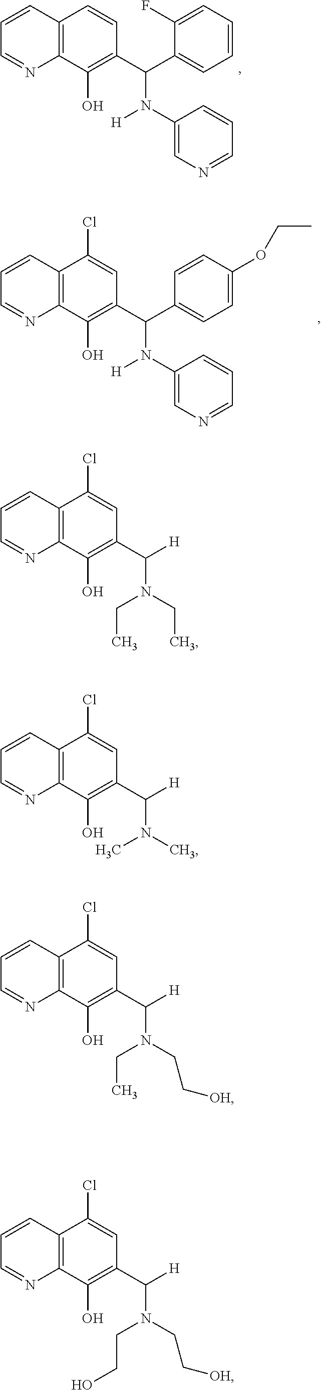

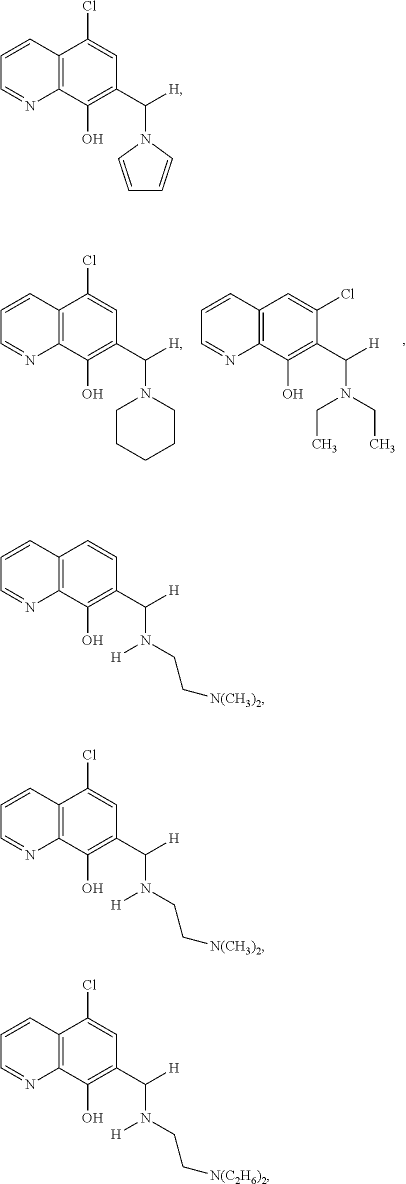

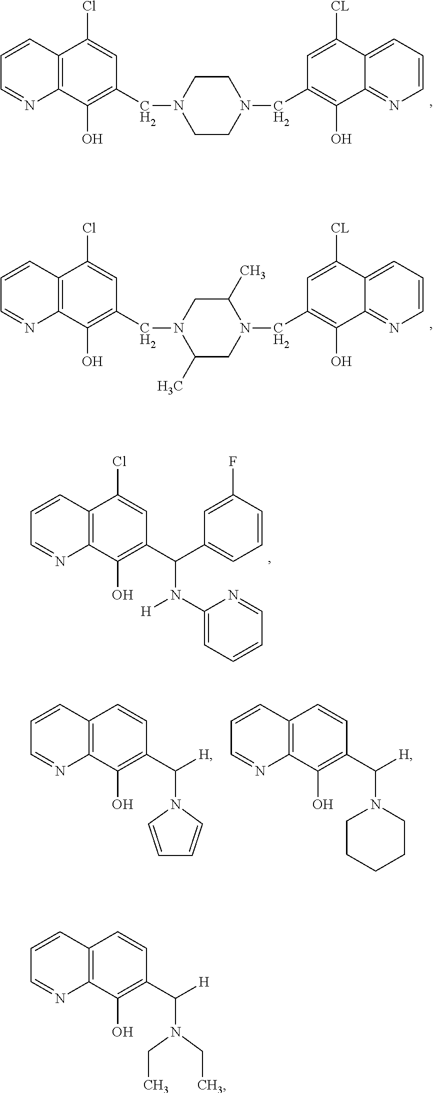

In a further more preferred embodiment of compounds of the general structural formula (I) for use as medicaments, the following compounds are moreover also excluded:

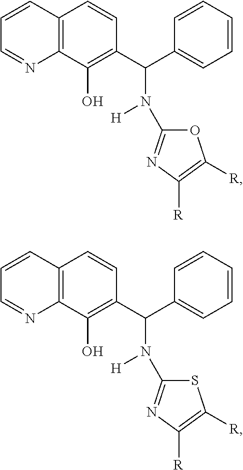

R=optionally substituted R=optionally substituted

The compounds according to the invention are therefore also suitable for the preparation of a medicament for treatment of patients suffering from symptoms of an iron deficiency anaemia, such as, for example: tiredness, lack of drive, lack of concentration, low cognitive efficiency, difficulties in finding the correct words, forgetfulness, unnatural pallor, irritability, accelerated heart rate (tachycardia), sore or swollen tongue, enlarged spleen, pregnancy cravings (pica), headaches, loss of appetite, increased susceptibility to infections, depressive moods or suffering from ACD or AL.

The compounds according to the invention are therefore also suitable for the preparation of a medicament for treatment of patients suffering from symptoms of an iron deficiency anaemia.

Administration can take place over a period of several months until the iron status improves, reflected, for example, by the haemoglobin value, the transferrin saturation and the ferritin value of the patient, or until the desired improvement is achieved in an impairment of the state of health caused by iron deficiency anaemia or by ACD or AI.

The preparation according to the invention can be taken by children, adolescents and adults.

The compounds of the present invention can furthermore also be used in combination with further active compounds or medicaments known in the treatment of disorders in iron metabolism and/or with active compounds or medicaments which are administered concomitantly with agents for treatment of diseases which are associated with disorders in iron metabolism, in particular with iron deficiency and/or anaemias. Examples of such agents for treatment of disorders in iron metabolism and further diseases associated with iron deficiency and/or anaemias which can be used in combination can include, for example, iron-containing compounds, such as e.g. iron salts, iron-carbohydrate complex compounds, such as iron-maltose or iron-dextrin complex compounds, vitamin D and/or derivatives thereof.

The compounds used in combination with the compounds according to the invention can be administered in this context either orally or parenterally, or the administration of the compounds according to the invention and of the compounds used in combination can take place by combination of the administration possibilities mentioned.

The compounds according to the invention and the combinations of the compounds according to the invention with further active compounds or medicaments can be employed in the treatment of disorders in iron metabolism, such as, in particular, iron deficiency diseases and/or anaemias, in particular anaemias with cancer, anaemia induced by chemotherapy, anaemia induced by inflammation (AI), anaemias with congestive cardiac insufficiency (CHF; congestive heart failure), anaemia with chronic renal insufficiency stage 3-5 (CKD 3-5; chronic kidney diseases stage 3-5), anaemia induced by chronic inflammation (ACD), anaemia with rheumatic arthritis (RA; rheumatoid arthritis), anaemia with systemic lupus erythematosus (SLE) and anaemia with inflammatory intestinal diseases (IBD; inflammatory bowel disease) or used for the preparation of medicaments for treatment of these diseases.

The compounds according to the invention and the above-mentioned combinations of the compounds according to the invention with further active compounds or medicaments can be used in particular for the preparation of medicaments for treatment of iron deficiency anaemia, such as iron deficiency anaemias in pregnant women, latent iron deficiency anaemia in children and adolescents, iron deficiency anaemia as a result of gastrointestinal abnormalities, iron deficiency anaemia as a result of blood losses, such as by gastrointestinal haemorrhages (e.g. as a result of ulcers, carcinomas, haemorrhoids, inflammatory disorders, intake of acetylsalicylic acid), menstruation, injuries, iron deficiency anaemia as a result of psilosis (sprue), iron deficiency anaemia as a result of reduced uptake of iron from the diet, in particular in selectively eating children and adolescents, weak immune system caused by iron deficiency anaemia, impaired cerebral performance caused by iron deficiency anaemia, restless leg syndrome.

The use according to the invention leads to an improvement in the iron, haemoglobin, ferritin and transferrin values which, especially in adolescents and children, but also in adults, are accompanies by an improvement in the short term memory test (STM), in the long term memory test (LTM), in the Raven's progressive matrices test, in the Wechsler adult intelligence scale (WAIS) and/or in the emotional coefficient (Baron EQ-i, YV test; youth version), or to an improvement in neutrophile levels, antibody levels and/or lymphocyte function.

The present invention furthermore relates to pharmaceutical compositions comprising one or more compounds of the formula (I) according to the invention and optionally one or more further pharmaceutically active compounds and optionally one or more pharmacologically acceptable carriers and/or auxiliary substances and/or solvents.

In this context, the pharmaceutical carriers, auxiliary substances or solvents are conventional substances. The pharmaceutical compositions mentioned are suitable, for example, for intravenous, intraperitoneal, intramuscular, intravaginal, intrabuccal, percutaneous, subcutaneous, mucocutaneous, oral, rectal, transdermal, topical, intradermal, intragastral or intracutaneous administration and are present, for example, in the form of pills, tablets, tablets resistant to gastric juice, film-coated tablets, layered tablets, sustained release formulations for oral, subcutaneous or cutaneous administration (in particular as patches), depot formulation, sugar-coated tablets, small suppositories, gels, ointments, syrup, granules, suppositories, emulsions, dispersions, microcapsules, microformulations, nanoformulations, liposomal formulations, capsules, capsules resistant to gastric juice, powders, powders for inhalation, microcrystalline formulations, sprays for inhalation, dusting powders, drops, nasal drops, nasal sprays, aerosols, ampoules, solutions, juices, suspensions, infusion solutions or injection solutions etc.

Preferably, the compounds according to the invention and pharmaceutical compositions comprising such compounds are administered orally and/or parenterally, in particular intravenously.

For this, the compounds according to the invention are preferably present in pharmaceutical compositions in the form of pills, tablets, tablets resistant to gastric juice, film-coated tablets, layered tablets, sustained release formulations for oral administration, depot formulations, sugar-coated tablets, granules, emulsions, dispersions, microcapsules, microformulations, nanoformulations, liposomal formulations, capsules, capsules resistant to gastric juice, powders, microcrystalline formulations, dusting powders, drops, ampoules, solutions, suspensions, infusion solutions or injection solutions.

The compounds according to the invention can be administered in a pharmaceutical composition which can comprise various organic or inorganic carriers and/or auxiliary materials such as are conventionally used for pharmaceutical purposes, in particular for solid medicament formulations, such as, for example, excipients (such as sucrose, starch, mannitol, sorbitol, lactose, glucose, cellulose, talc, calcium phosphate, calcium carbonate), binders (such as cellulose, methylcellulose, hydroxypropylcellulose, polypropylpyrrolidone, gelatine, gum arabic, polyethylene glycol, sucrose, starch), disintegrating agents (such as starch, hydrolysed starch), carboxymethylcellulose, calcium salt of carboxymethylcellulose, hydroxypropyl-starch, sodium glycol starch, sodium bicarbonate, calcium phosphate, calcium citrate), lubricants and slip agents (such as magnesium stearate, talc, sodium lauryl sulfate) a flavouring agent (such as citric acid, menthol, glycine, orange powder), preservatives (such as sodium benzoate, sodium bisulfite, methylparaben, propylparaben), stabilizers (such as citric acid, sodium citrate, acetic acid, and multicarboxylic acids from the Titriplex series, such as e.g. diethylenetriaminepentaacetic acid (DTPA), suspending agents (such as methylcellulose, polyvinylpyrrolidone, aluminium stearate), dispersing agents, diluents (such as water, organic solvents), beeswax, cacao butter, polyethylene glycol, white petrolatum etc.

Liquid medicament formulations, such as solutions, suspensions and gels, conventionally contain a liquid carrier, such as water and/or pharmaceutically acceptable organic solvents. Such liquid formulations can furthermore also contain pH-adjusting agents, emulsifiers or dispersing agents, buffering agents, preservatives, wetting agents, gelling agents (for example methylcellulose), colouring agents and/or aroma substances. The compositions can be isotonic, that is to say these can have the same osmotic pressure as blood. The isotonicity of the composition can be adjusted using sodium chloride or other pharmaceutically acceptable agents, such as, for example, dextrose, maltose, boric acid, sodium tartrate, propylene glycol or other inorganic or organic soluble substances. The viscosity of the liquid compositions can be adjusted using a pharmaceutically acceptable thickening agent, such as methylcellulose, Other suitable thickening agents include, for example, xanthan, carboxymethylcellulose, hydroxypropylcellulose, carbomer and the like. The preferred concentration of the thickening agent will depend on the agent chosen. Pharmaceutically acceptable preservatives can be used to increase the life of the liquid composition. Benzyl alcohol may be suitable, although a large number of preservatives, including, for example, paraben, thimerosal, chlorobutanol or benzalkonium chloride, can likewise be used.

The active compound can be administered with a unit dose of from 0.001 mg/kg to 500 mg/kg of body weight, for example up to 1 to 4 times a day. However, the dosage can be increased or reduced, depending on the age, weight, condition of the patient, severity of the disease or nature of the administration.

Particular embodiments of the invention relate to:

1. Compounds of the general formula (I)

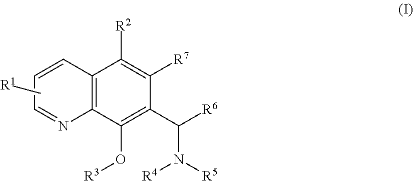

wherein
R¹, R² and R⁷ are identical or different and are each chosen from the group consisting of:
hydrogen,
hydroxyl,
halogen,
cyano,
nitro,
carboxyl,
sulfonic acid radical (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heteroaryl;
   R⁶ is chosen from the group consisting of:
hydrogen,
optionally substituted alkyl,
optionally substituted alkenyl,
optionally substituted alkynyl,
optionally substituted aryl,
optionally substituted heteroaryl;
   R³ is chosen from the group consisting of:
hydrogen,
optionally substituted alkyl,
optionally substituted alkenyl,
optionally substituted alkynyl,
optionally substituted acyl,
optionally substituted aryl,
optionally substituted heteroaryl; and
   R⁴ and R⁵ are identical or different and are each chosen from the group consisting of:
hydrogen,
optionally substituted alkyl,
optionally substituted alkenyl,
optionally substituted alkynyl,
optionally substituted acyl,
optionally substituted aryl,
optionally substituted heteroaryl, or
wherein R⁴ and R⁵ together with the nitrogen atom to which they are bonded form a saturated or unsaturated, optionally substituted 5- to 8-membered ring which can optionally contain further hetero atoms;
   or pharmaceutically acceptable salts thereof.

2. Compounds according to embodiment 1, wherein

R¹, R² and R⁷ are identical or different and are each chosen from the group consisting of:
hydrogen,
hydroxyl,
halogen,
optionally substituted alkyl,
optionally substituted alkoxy,
optionally substituted aryl,
optionally substituted heteroaryl;
   R⁶ is chosen from the group consisting of:
hydrogen,
optionally substituted alkyl,
optionally substituted aryl,
optionally substituted heteroaryl;
   R³ is chosen from the group consisting of:
hydrogen,
optionally substituted alkyl,
optionally substituted aryl,
optionally substituted heteroaryl;
optionally substituted acyl; and
   R⁴ and R⁵ are identical or different and are each chosen from the group consisting of:
hydrogen,
optionally substituted alkyl,
optionally substituted aryl,
optionally substituted heteroaryl,
   or R⁴ and R⁵ together with the nitrogen atom to which they are bonded form a saturated or unsaturated, optionally substituted 5- to 8-membered ring which can optionally contain further hetero atoms;
   or pharmaceutically acceptable salts.

3. Compounds according to embodiment 1 or 2, wherein

R¹, R² and R⁷ are identical or different and are each chosen from the group consisting of:
hydrogen,
hydroxyl,
halogen,
optionally substituted alkyl,
optionally substituted alkoxy;
   R⁶ is chosen from the group consisting of:
hydrogen,
optionally substituted alkyl,
optionally substituted aryl,
optionally substituted heteroaryl;
   R³ is chosen from the group consisting of:
hydrogen,
optionally substituted alkyl,
optionally substituted acyl; and
   R⁴ and R⁵ are identical or different and are each chosen from the group consisting of:
hydrogen,
optionally substituted aryl,
optionally substituted heteroaryl,
   or wherein R⁴ and R⁵ together with the nitrogen atom to which they are bonded form a saturated or unsaturated, optionally substituted 5- to 6-membered ring which can optionally contain further hetero atoms;
   or pharmaceutically acceptable salts thereof.

4. Compounds according to one or more of embodiments 1 to 3, with the meanings:

### R¹:

hydrogen;

### R²:

hydrogen or
halogen;

### R³:

hydrogen, or
optionally substituted acyl, in particular optionally substituted aroyl or optionally substituted heteroaroyl;
   R⁴ and R⁵ are identical or different and denote:
hydrogen,
optionally substituted aryl,
optionally substituted heteroaryl, or
R⁴ and R⁵ together with the nitrogen atom to which they are bonded form a saturated or unsaturated, optionally substituted 6-membered ring which can contain further hetero atoms.

### R⁶:

hydrogen,
optionally substituted aryl, or
optionally substituted heteroaryl; and

### R⁷:

hydrogen;
   or pharmaceutically acceptable salts thereof.

5. Compounds according to one or more of embodiments 1 to 4, wherein

R¹ is hydrogen,
R² is chosen from
hydrogen or
chlorine;
   R³ is chosen from
hydrogen, or
optionally substituted furoyl or optionally substituted benzoyl;
   R⁴ and R⁵ are identical or different and denote:
hydrogen,
optionally substituted heteroaryl, or
R⁴ and R⁵ together with the nitrogen atom to which they are bonded form a saturated, optionally substituted 6-membered ring which contains a further hetero atom;
   R⁶ denotes:
hydrogen,
optionally halogen-substituted phenyl or
pyridinyl; and
   R⁷ is hydrogen,
   or pharmaceutically acceptable salts thereof.

6. Compounds according to one or more of embodiments 1, 2 or 3, wherein at least one of the substituents R¹, R², R³, R⁴, R⁵, R⁶ and R⁷ is as defined in embodiment 4 or 5.

7. Compounds according to one or more of embodiments 1 to 6, chosen from

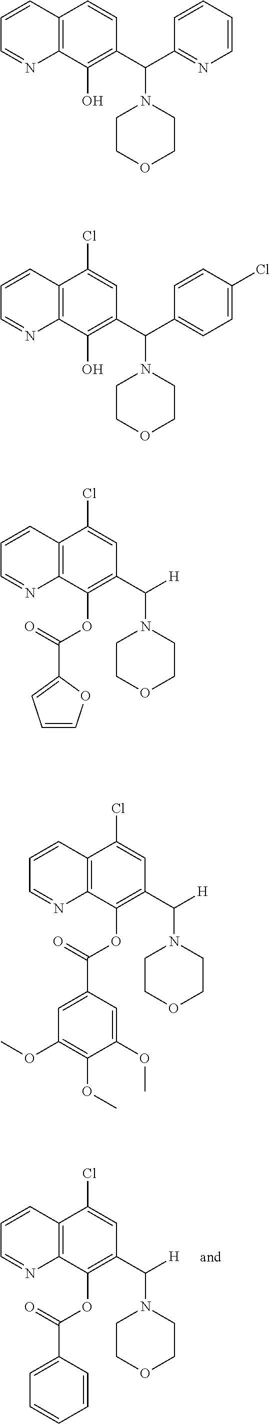

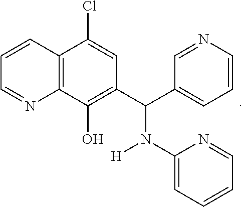

or pharmaceutically acceptable salts thereof.

8. Process for the preparation of the compounds according to one or more of embodiments 1 to 7, which comprises the steps:
a) reaction of the compound of the formula (II)

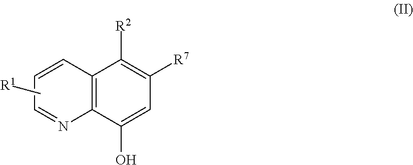

wherein R¹, R² and R⁷ have the abovementioned meanings,
with compounds of the formulae (III) and (IV):

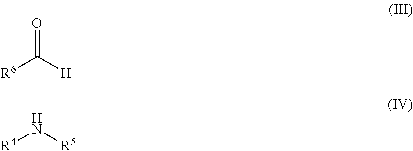

wherein R⁴, R⁵ and R⁶ have the abovementioned meanings,
in the sense of an aminoalkylation to give compounds of the formula (I′):

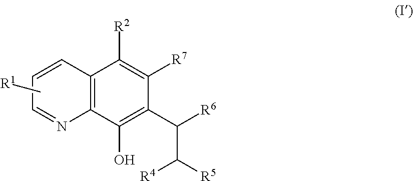

and
(b) reaction of the compound of the formula (I′) with a compound of the formula

R³—X,
wherein R³ is as defined above, and X is a usual leaving group, such as halogen, to give compounds of the formula (I).

9. Compounds according to one or more of embodiments 1 to 7 for use as medicaments.

10. Compounds according to one or more of embodiments 1 to 7 for use in the treatment of disorders in iron metabolism, in particular for use for iron deficiency diseases and/or anaemias, in particular anaemias with cancer, anaemia induced by chemotherapy, anaemia induced by inflammation (AI), anaemias with congestive cardiac insufficiency (CHF; congestive heart failure), anaemia with chronic renal insufficiency stage 3-5 (CKD 3-5; chronic kidney diseases stage 3-5), anaemia induced by chronic inflammation (ACD), anaemia with rheumatic arthritis (RA; rheumatoid arthritis), anaemia with systemic lupus erythematosus (SLE) and anaemia with inflammatory intestinal diseases (IBD; inflammatory bowel disease).

11. Composition comprising one or more of the compounds according to one or more of embodiments 1 to 7 and one or more pharmaceutical carriers and/or auxiliary substances and/or solvents

12. Combination preparation comprising one or more of the compounds according to one or more of embodiments 1 to 7 and at least one further pharmaceutically active compound, in particular a compound for treatment of disorders in iron metabolism and the accompanying symptoms, preferably an iron-containing compound.

13. Use of the compounds according to one or more of embodiments 1 to 7, of the composition according to embodiment 11 and of the combination preparation according to embodiment 12 for the preparation of a medicament for treatment of hepcidin-mediated diseases and the accompanying symptoms.

14. Use of the compounds according to one or more of embodiments 1 to 7, of the composition according to embodiment 11 and of the combination preparation according to embodiment 12 for the preparation of a medicament for treatment of disorders in iron metabolism, in particular iron deficiency diseases and/or anaemias, in particular ACD and AI and the accompanying symptoms.

15. Use of the compounds according to one or more of embodiments 1 to 7, of the composition according to embodiment 11 and of the combination preparation according to embodiment 12 for the preparation of a medicament for oral or parenteral administration.

The invention is illustrated in more detail by the following examples. The examples are given merely by way of example and the person skilled in the art is in a position to extend the specific examples to further compounds claimed.

### EXAMPLES

### Pharmacological Action Studies

The following materials were used:

| Reagents | Batch no. | Comments |
|---|---|---|
| MDCK-FPN-HaloTag clone 7 | | |
| Hepcidin 100 μM stock | Lot# 571007 | Peptides International |
| solution in water | | |
| HaloTag ®TMR ligand | Lot# 257780 | Promega, cat# G8251 |
| Opera confocal plate | | PerkinElmer |
| imager | | |
| Perkin Elmer 384 Cell carrier | | cat# 6007430 |
| plates | | |
| Paraformaldehyde | Lot# 080416 | Electron Microscopy |
| | | Sciences |
| | | cat# 15710-S |
| Draq5 | | Biostatus, cat no: |
| | | DR51000 |

The hepcidin-antagonistic action of the quinoline compounds of the present invention was determined by means of the “ferroportin internalization assay” described in the following.

### Principle of the “Ferroportin Internalization Assay”

Organic compounds of low molecular weight which counteract the biological actions of hepcidin on its receptor, the iron exporter ferroportin (Fpn), were identified on the basis of their ability to inhibit hepcidin-induced internalization of Fpn in living cells. For this purpose, a stable cell line (Madin-Darby canine kidney, MDCK) was produced which constitutively expresses human ferroportin fused recombinantly at its C terminus with a fluorescent reporter protein (HaloTag®, Promega Corp.). The internalization of Fpn was monitored by labelling these cells with fluorescent ligands (HaloTag®-TMR, tetramethylrhodamine) which join covalently on to the HaloTag reporter gene fused with the Fpn. Imaging by confocal fluorescence microscopy showed a cell surface location of Fpn in the absence of hepcidin and the absence of Fpn surface staining in the presence of hepcidin. Optimized image analysis algorithms were used to ascertain the cell surface and to quantify the corresponding membrane fluorescence associated with the Fpn-HaloTag fusion protein. This assay allows a quantitative image-based analysis in order to quickly evaluate compounds which can block hepcidin-induced internalization of Fpn. This assay is a direct in vitro pendant of the in vivo action mechanism proposed for medicament candidates and is therefore suitable as an initial assay with a high throughput for identifying compounds which counteract the action of hepcidin on its receptor ferroportin.

### Detailed Assay Procedure

7,500 cells per well (MDCK-FPN-HaloTag) were transinoculated in 50 μl DMEM medium (Dulbeccos modified Eagle medium with 10% foetal bovine serum (FBS), which contained 1% penicillin, 1% streptomycin and 450 μg/ml of G-418) in microtitre plates with 384 wells, (384 Cell carrier plates, Perkin Elmer, cat. no. 6007430), followed by incubation overnight at 37° C./5% CO₂.
The volume of the medium was reduced to 10 μl, and 10 μl of 5 μM HaloTag-TMR ligands (Promega, cat. no. G 8251) were added in DMEM medium in order to stain the Fpn-HaloTag fusion protein.
15 min incubation at 37° C./5% CO₂
The HaloTag-TMR ligand was removed and the cells were washed with fresh DMEM medium and the volume was reduced to 20 μl of DMEM medium.
3 μl per well of a solution of the test compound (dissolved DMSO) were added (10 μl final volume).
7 μl of 43 μM hepcidin (Peptides International, cat. no. PLP-4392-s, 100 μM stock solution diluted in water in DMEM medium) were added per well up to a final hepcidin concentration of 100 nM.
The cells were incubated overnight at 37° C./5% CO₂.
The cells were fixed by adding paraformaldehyde (PFA, Electron Microscopy Sciences, cat. no. 15710-S) directly to the cells up to a final concentration of 4%, followed by incubation at room temperature for 15-20 minutes.
The PFA solution was removed and the cells were washed with PBS (phosphate-buffered saline solution), in each case 30 μl remaining in the plate.
20 μl of Draq5 (Biostatus, cat. no. DR 51000) were added up to a final concentration of 2.5 μM in order to stain the cell nuclei, and the plates were sealed with a foil plate seal.
The plates were analysed with the Opera Plate Imager (Opera Confocal Plate Imager, Perkin Elmer) with 7 images per well; 440 ms exposure time per image, 1 μM focal point height.

### Analysis of the Data

Optimized algorithms were used for the image analysis to ascertain and quantify the fluorescence associated with the cell surface as a measure of the cell surface location of Fpn-HaloTag.
The final display corresponded to the percentage content of cells which showed membrane fluorescence: wells treated with 1000 nM hepcidin gave the lowest values (negative control display=0% inhibition of the Fpn internalization) and wells which were not treated with hepcidin resulted in the maximum percentage content of cells with membrane fluorescence (positive control display 100% inhibition of the Fpn internalization).
On each plate, the median value of the 6 positive and 6 negative control values was used to calculate the percentage inhibition of the compounds tested according to the following formula:

$$I = 100 \times \frac{{R}_{neg} - {R}_{compound}}{{R}_{neg} - {R}_{pos}}$$

where:
   Rₚₒₛ positive control display value (median)
   R_{neg} negative control display value (median)
   R_{compound} display value of the compound investigated
   I percentage inhibition by the particular compound
In dose/effect studies, dilution series (11 concentrations, 1:2 dilution steps) of the compounds were tested (concentration range from 0.04 to 40 μM), and standardized signal values of replicated tests (average of 6 titrations on independent plates) were used to fit the curves by a robust standard dose/effect model with four parameters (lower asymptote, upper asymptote, IC50, gradient).

The following results were obtained:

| | | | I [%] |
|---|---|---|---|
| | | | (median |
| | | | in- |
| | | | hibition |
| | | | [%] at |
| | | Ferro- | 10 μM |
| | | portin | sub- |
| Ex- | | IC50 | stance |
| ample | Compound | [μM] | conc.) |
| 1 | 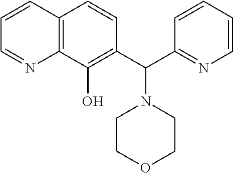 | <50 | >50 |
| 2 | 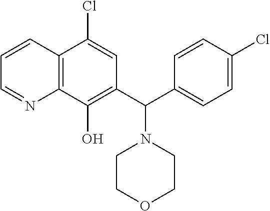 | <50 | <50 |
| 3 | 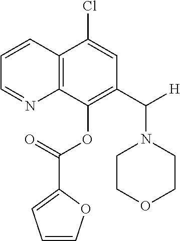 | <50 | <50 |
| 4 | 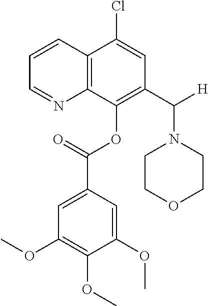 | <50 | <50 |
| 5 | 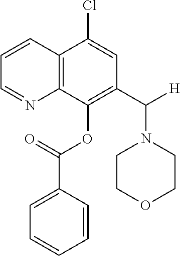 | <50 | <50 |
| 6 | 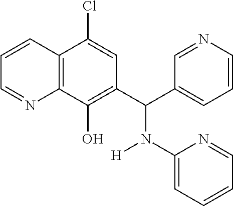 | <50 | <50 |
| 7 | 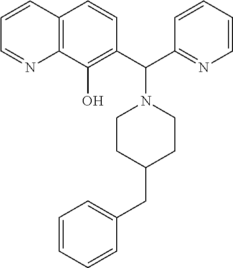 | <50 | |
| 8 | 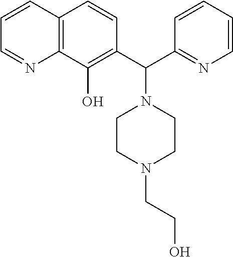 | <50 | |
| 9 | 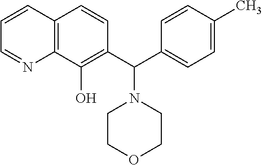 | <50 | |
| 10 | 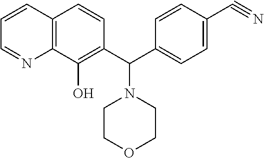 | <50 | |
| 11 | 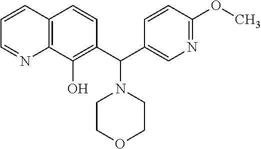 | <50 | |
| 12 | 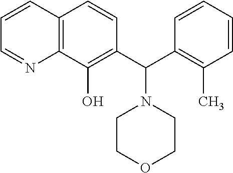 | <50 | |
| 13 | 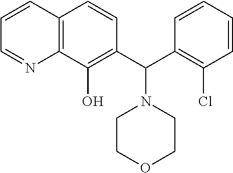 | <50 | |
| 14 | 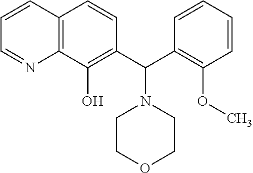 | <50 | |
| 15 | 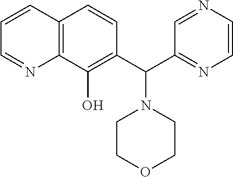 | <50 | |
| 16 | 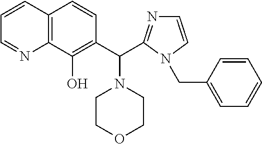 | <50 | |
| 17 | 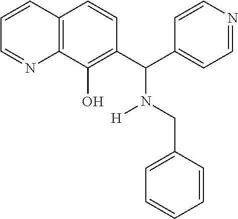 | <50 | |
| 18 | 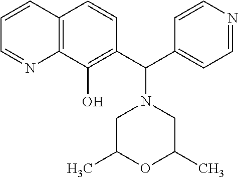 | <50 | |
| 19 | 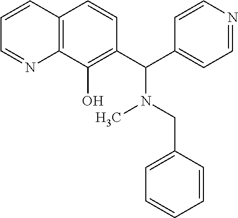 | <50 | |
| 20 | 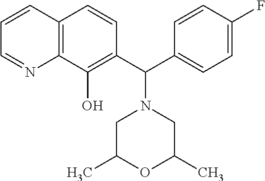 | <50 | |
| 21 | 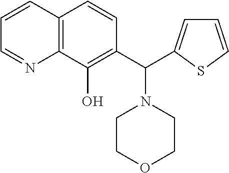 | <50 | |
| 22 | 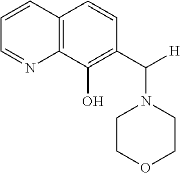 | <50 | |
| 23 | 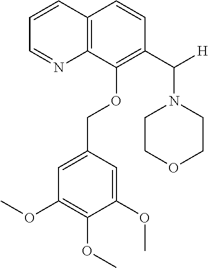 | >100 | |
| 24 | 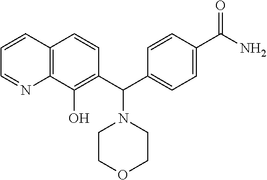 | <50 | |
| 25 | 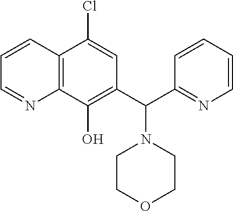 | <50 | |
| 26 | 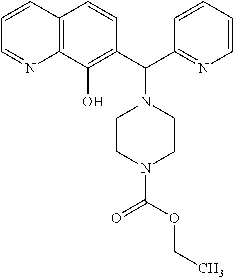 | <50 | |
| 27 | 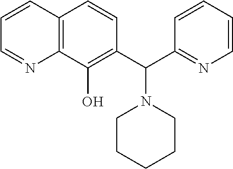 | <50 | |
| 28 | 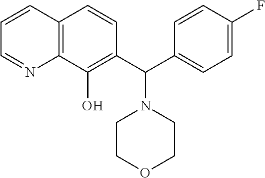 | <50 | |
| 29 | 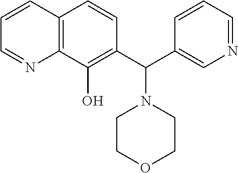 | <50 | |
| 30 | 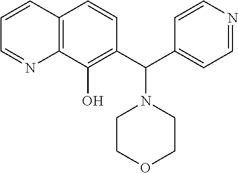 | <50 | |
| 31 | 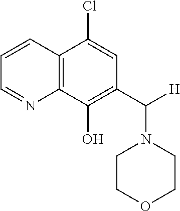 | <50 | |
| 32 | 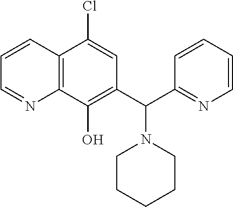 | <50 | |
| 33 | 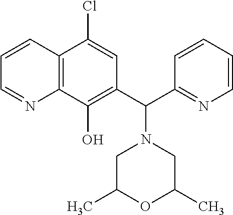 | <50 | |
| 34 | 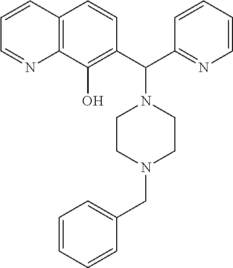 | <50 | |
| 35 | 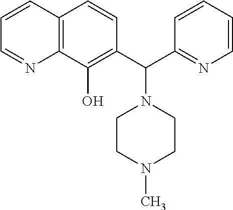 | <50 | |
| 36 | 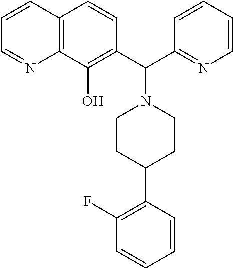 | <50 | |
| 37 | 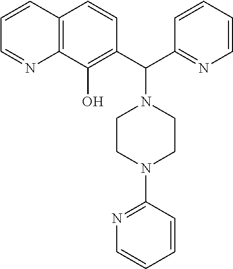 | <50 | |
| 38 | 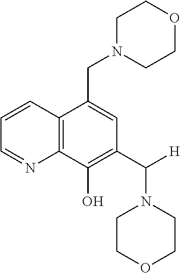 | >100 | |
| 39 | 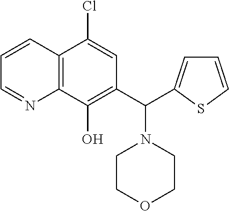 | <50 | |
| 40 | 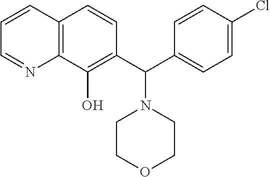 | <50 | |
| 41 | 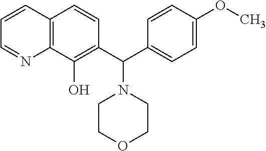 | <50 | |

### Preparation Examples

### A) Purification by Means of Preparative HPLC and Column Chromatography

The following preparation examples were carried out according to the preparation process according to the invention with subsequent purification by means of preparative HPLC and/or by means of column chromatography under the following conditions:

### Preparative HPLC (Neutral Conditions):

Method: Gilson semi-prep HPLC with 119 UV detector and 5.11 Unipoint Control Software

Stationary phase/column: Waters SunFire Prep C18 OBD (5 μm, 19×100 mm), room temperature

Mobile phase:
A—water
B—acetonitrile

Flow rate: 20 ml/min

Injection volume: 1,000

Detection: UV

Eluent:

| Time (min) | Solvent |
|---|---|
| 0.0-2.0 | 5% B + 95% A |
| 2.0-2.5 | constant gradient to 10% B + 90% A |
| 2.5-14.5 | constant gradient to 100% B |
| 14.5-16.5 | 100% B |
| 16.5-16.7 | constant gradient to 5% B + 95% A |
| 16.7-17.2 | 5% B + 95% A |

### Column Chromatography:

The “flash” silica gel chromatography was carried out by means of silica gel 230-400 mesh or on prepacked silica gel columns.

### B) Analytical HPLC

The detection and determination of the purity of the compounds were in each case carried out by means of HPLC MS (high performance liquid chromatography with mass spectrometry (MS)) or by means of HPLC with UV detection (PDA; photo diode array).

The following method was used here:

Method: MS19_{—}7MIN_HIRES_POS/high resolution method

Stationary phase/column: Waters Atlantis dC18 100×2.1 mm, 3 μm column, 40° C.

Mobile phase:
A—0.1% formic acid (water)
A—0.1% formic acid (acetonitrile)

Flow rate: 0.6 ml/min

Injection volume: 3 μl

UV detector: 215 nm (nominal)

or

MS detection: TIC (total ion count)

| | | |
|---|---|---|
| | | Organic |
| | Time | content |
| Gradient | (min) | (%) |

| | |
|---|---|
| 0.00 | 5 |
| 5.00 | 100 |
| 5.40 | 100 |
| 5.42 | 5 |

| Time (min) | Solvent |
|---|---|
| 0.0-5.0 | constant gradient from 95% A + 5% B to |
| | 100% B |
| 5.0-5.4 | 100% B |
| 5.4-5.42 | constant gradient from 100% B to 95% A + |
| | 5% B |
| 5.42-7.0 | 95% A + 5% B |

HPLC-MS system: Shimadzu LCMS 2010EV system

Mass range: 100-1,000 m/z

Scan speed: 2,000 amu/sec

### Compound According to Example 1

### 7-(Morpholin-4-yl-pyridin-2-yl-methyl)-quinolin-8-ol

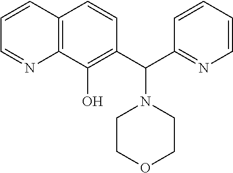

EOAI3333419 VIT-1197/1003

MW: 321.38

Manufacturer: Enamine

HPLCMS: [m/z]: 322

FIG. 1 shows the result.

### Compound According to Example 2

### 5-Chloro-7-[(4-chloro-phenyl)-morpholin-4-yl-methyl]-quinolin-8-ol

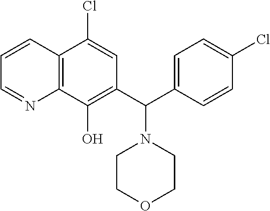

EOAI3333421 VIT-1005

MW: 389.2

Manufacturer: Enamine

HPLCMS: [m/z]: 389

FIG. 2 shows the result.

### Compound According to Example 3

### Furan-2-carboxylic acid 5-chloro-7-morpholin-4-ylmethyl-quinolin-8-yl ester

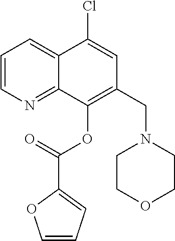

EOAI3333420 VIT-1004

MW: 372.8

Manufacturer: Enamine

HPLCMS: [m/z]: 372.9

FIG. 3 shows the result.

### Compound According to Example 4

### 3,4,5-Trimethoxy-benzoic acid 5-chloro-7-morpholin-4-ylmethyl-quinolin-8-yl ester

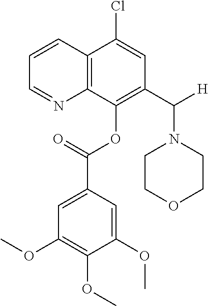

EOAI3325565 VIT-1028

MW: 472.93

Manufacturer: Enamine

HPLCMS: [m/z]: 473

FIG. 4a shows the result.

In addition, Example Compound 4 was analysed analogously by means of analytical HPLC as a commercially obtainable compound.

HP-AH002047-G10

MW: 472.92

Manufacturer: ENAMINE

UV spectrum: λ max [ηm]: 213, 283

HPLCMS: [m/z]: 473

HPLCMS: [m/z]: 537

This MS-chromatogram shows an impurity of approx. 10-13% at MW 537

FIG. 4b shows the result.

### Compound According to Example 5

### Benzoic acid 5-chloro-7-morpholin-4-ylmethyl-quinolin-8-yl ester

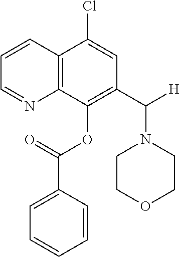

5-Chloroquinolin-8-ol (899 mg, 5.0 mmol), formaldehyde (37% aqueous solution, 580 μl) and morpholine (437 μl, 5.0 mmol) were dissolved in ethanol (30 ml) and the mixture was stirred at room temperature for 24 hours. The mixture was then heated at 80° C. for 6.5 hours. After cooling, the mixture was filtered and the filtrate was concentrated in vacuo. The crude material obtainable in this way was purified by means of column chromatography with methanol/methylene chloride (0-20%) as the eluent to give 5-chloro-7-morpholin-4-ylmethyl-quinolin-8-ol (1.02 g, 73%).

5-Chloro-7-morpholin-4-ylmethyl-quinolin-8-ol (278 mg, 1.0 mmol), benzoyl chloride (115 μl, 1.0 mmol) and triethylamine (TEA) (140 μl, 1.0 mmol) were dissolved in tetrahydrofuran (THF) (5 ml) and the mixture was stirred at room temperature for 2 hours. The mixture was concentrated in vacuo and the yield was extracted by means of water/methylene chloride. The phases were separated and the organic phase was concentrated in vacuo. The yield obtainable in this way was purified by means of column chromatography with ethyl acetate/heptane (0-20%) as the eluent to give Example Compound 5 (298 mg, 78%).

EOAI3328875 VIT-1075

MW: 382.85

HPLCMS: [m/z]: 383

FIG. 5 shows the result.

### Compound According to Example 6

### 5-Chloro-7-[pyridin-3-yl-(pyridin-2-ylamino)-methyl]-quinolin-8-ol

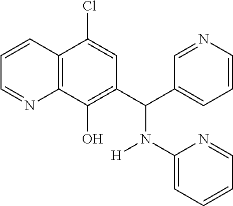

1,5-Chloroquinolin-8-ol (180 mg, 1.0 mmol), 2-pyridinecarboxaldehyde (189 μl, 2.0 mmol) and 2-aminopyridine (85 mg, 0.9 mmol) were dissolved in dioxane (2 ml) and the mixture was heated in a microwave oven at 118° C. for 10 minutes. After cooling, the mixture was concentrated in vacuo.

Purification was carried out by means of column chromatography ×3 with 2 M methanolic ammonia/methylene chloride (0-20%) as the eluent, followed by trituration from acetone to give Example Compound 6 (16 mg, 5%).

EOAI3284909 VIT-1039

MW: 362.82

HPLCMS: [m/z]: 362

FIG. 6a shows the result.

In addition, Example Compound 6 was analysed analogously by means of analytical HPLC as a commercially obtainable compound.

HP-AT003015-E05

MW: 362.81

Manufacturer: CHEMBRIDGE

UV spectrum: λ max [ηm]: 197, 248, 309

HPLCMS: [m/z]: 362

FIG. 6b shows the result.

### Compound According to Example 7

### 7-[(4-Benzyl-piperidin-1-yl)-pyridin-2-yl-methyl]-quinolin-8-ol

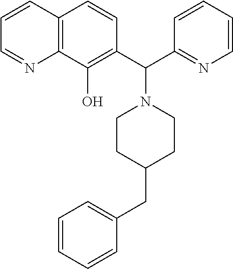

The preparation was carried out analogously to the method described for Example Compound 6 using:
8-hydroxyquinoline (145 mg, 1.0 mmol)
2-pyridinecarboxaldehyde (95 μl, 1.0 mmol) and
4-benzylpiperidine (176 μl, 1.0 mmol)
in iso-propyl alcohol (2 ml)

Purification was carried out by means of preparative HPLC to give Example Compound 7 (18 mg, 11%).

EOAI3330062 VIT-1082

MW: 409.53

HPLCMS: [m/z]: 410

FIG. 7 shows the result.

### Compound According to Example 8

### 7-{[4-(2-Hydroxy-ethyl)-piperazin-1-pyridin-2-yl-methyl}-quinolin-8-ol

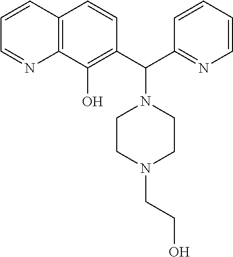

The preparation was carried out analogously to the method described for Example Compound 6 using:
8-hydroxyquinoline (145 mg, 1.0 mmol)
2-pyridinecarboxaldehyde (95 μl, 1.0 mmol) and
2-(piperidin-4-yl)ethanol (123 μl, 1.0 mmol)
in iso-propyl alcohol (2 ml)

Purification was carried out by means of preparative HPLC to give Example Compound 8 (27 mg, 19%).

EOAI3330063 VIT-1083

MW: 364.45

HPLCMS: [m/z]: 365

FIG. 8 shows the result.

### Compound According to Example 9

### 7-(Morpholin-4-yl-p-tolyl-methyl)-quinolin-8-ol

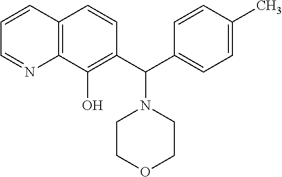

The preparation was carried out analogously to the method described for Example Compound 6 using:
8-hydroxyquinoline (290 mg, 2.0 mmol)
4-methylbenzaldehyde (474 μl, 4.0 mmol)
and morpholine (157 μl, 1.8 mmol)
in dioxane (2 ml).

Purification was carried out by means of column chromatography with 7 M methanolic ammonia/methylene chloride (0-5%) as the eluent, followed by preparative HPLC to give Example Compound 9 (18 mg, 3%).

EOAI3333335 VIT-1182

MW: 334.42

HPLCMS: [m/z]: 335

FIG. 9 shows the result.

### Compound According to Example 10

### 4-[(8-Hydroxy-quinolin-7-yl)-morpholin-4-yl-methyl]-benzonitrile

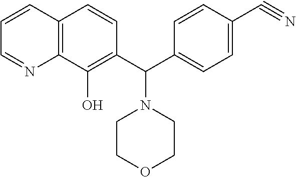

The preparation was carried out analogously to the method described for Example Compound 6 using:
8-hydroxyquinoline (290 mg, 2.0 mmol)
4-cyanobenzaldehyde (525 μl, 4.0 mmol)
and morpholine (157 μl, 1.8 mmol)
in dioxane (2 ml).

Purification was carried out by means of column chromatography with 7 M methanolic ammonia/methylene chloride (0-20%) as the eluent, followed by preparative HPLC to give Example Compound 10 (100 mg, 16%).

EOAI3333336 VIT-1181

MW: 345.40

HPLCMS: [m/z]: 345.9

FIG. 10 shows the result.

### Compound According to Example 11

### 7-[(6-Methoxy-pyridin-3-yl)-morpholin-4-yl-methyl]-quinolin-8-ol

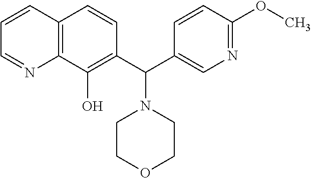

The preparation was carried out analogously to the method described for Example Compound 6 using:
8-hydroxyquinoline (290 mg, 2.0 mmol)
6-methoxypyridine-3-carbaldehyde (549 μl, 4.0 mmol)
and morpholine (157 μl, 1.8 mmol)
in dioxane (2 ml).

Purification was carried out by means of preparative HPLC to give Example Compound 11 (64 mg, 10%).

EOAI3333471 VIT-1192

MW: 351.41

HPLCMS: [m/z]: 352

FIG. 11 shows the result.

### Compound According to Example 12

### 7-(Morpholin-4-yl-o-tolyl-methyl)-quinolin-8-ol

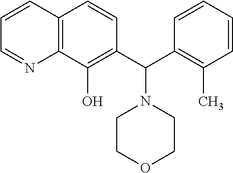

The preparation was carried out analogously to the method described for Example Compound 6 using:
8-hydroxyquinoline (290 mg, 2.0 mmol)
2-methylbenzaldehyde (463 μl, 4.0 mmol)
and morpholine (157 μl, 1.8 mmol)
in dioxane (2 ml).

Purification was carried out by means of preparative HPLC to give Example Compound 12 (77 mg, 13%).

EOAI3333472 VIT-1193

MW: 334.42

HPLCMS: [m/z]: 335

FIG. 12 shows the result.

### Compound According to Example 13

### 7-[(2-Chloro-phenyl)-morpholin-4-yl-methyl]-quinolin-8-ol

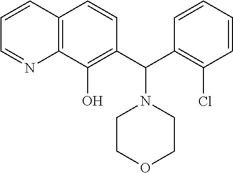

The preparation was carried out analogously to the method described for Example Compound 6 using:
8-hydroxyquinoline (290 mg, 2.0 mmol)
2-chlorobenzaldehyde (563 μl, 4.0 mmol)
and morpholine (157 μl, 1.8 mmol)
in dioxane (2 ml).

Purification was carried out by means of preparative HPLC to give Example Compound 13 (18 mg, 2%).

EOAI3333474 VIT-1195

MW: 354.84

HPLCMS: [m/z]: 355

FIG. 13 shows the result.

### Compound according to Example 14

### 7-[(2-Methoxy-phenyl)-morpholin-4-yl-methyl]-quinolin-8-ol

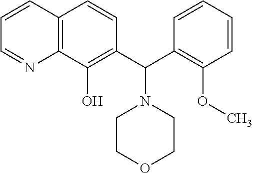

The preparation was carried out analogously to the method described for Example Compound 6 using:
8-hydroxyquinoline (290 mg, 2.0 mmol)
2-methoxybenzaldehyde (544 μl, 4.0 mmol)
and morpholine (157 μl, 1.8 mmol)
in dioxane (2 ml).

Purification was carried out by means of preparative HPLC to give Example Compound 14 (49 mg, 8%).

EOAI3333574 VIT-1206

MW: 350.42

HPLCMS: [m/z]: 351

FIG. 14 shows the result.

### Compound according to Example 15

### 7-(Morpholin-4-yl-pyrazin-2-yl-methyl)-quinolin-8-ol

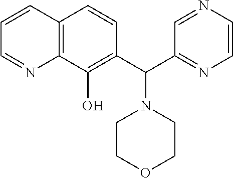

The preparation was carried out analogously to the method described for Example Compound 6 using:
8-hydroxyquinoline (145 mg, 1.0 mmol)
pyrazine-2-carbaldehyde (108 mg, 1.0 mmol)
and morpholine (79 μl, 0.9 mmol)
in dioxane (2 ml).

Purification was carried out by means of preparative HPLC to give Example Compound 15 (91 mg, 28%).

EOAI3333578 VIT-1207

MW: 322.37

HPLCMS: [m/z]: 323

FIG. 15 shows the result.

### Compound according to Example 16

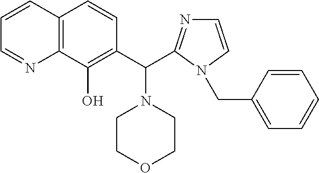

The preparation was carried out analogously to the method described for Example Compound 6 using:
8-hydroxyquinoline (251 mg, 1.72 mmol)
1-benzyl-1H-imidazole-2-carbaldehyde (644 mg, 3.45 mmol)
and morpholine (136 μl, 1.56 mmol)
in dioxane (3 ml).

Purification was carried out by means of preparative HPLC to give Example Compound 16 (270 mg, 43%).

EOAI3334110 VIT-1242

MW: 400.48

HPLCMS: [m/z]: 401

FIG. 16 shows the result.

### Compound according to Example 17

### 7-(Benzylamino-pyridin-4-yl-methyl)-quinolin-8-ol

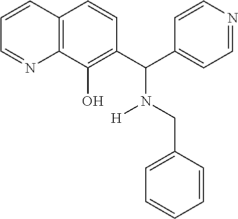

8-Hydroxyquinoline (150 mg, 1.0 mmol), 4-pyridinecarboxaldehyde (116 mg, 1.0 mmol) and benzylamine (199 mg, 2.0 mmol) were dissolved in ethanol (5 ml) and the mixture was heated under reflux for 18 hours. After cooling, the mixture was concentrated in vacuo to give the crude product.

Purification was carried out by means of preparative HPLC to give Example Compound 17 (62 mg, 18%).

EOAI3333571 VIT-1205

MW: 341.42

HPLCMS: [m/z]: 342

FIG. 17 shows the result.

### Compound according to Example 18

### 7-[(2,6-Dimethyl-morpholin-4-yl)-pyridin-4-yl-methyl]-quinolin-8-ol

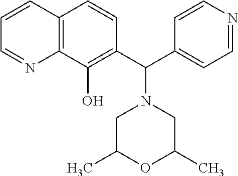

The preparation was carried out analogously to the method described for Example Compound 17 using:
8-hydroxyquinoline (250 mg, 2.0 mmol)
4-pyridinecarboxaldehyde (194 mg, 2.0 mmol)
and 2,6-dimethylmorpholine (357 mg, 3.0 mmol)

Purification was carried out by means of preparative HPLC to give Example Compound 18 (60 mg, 10%).

EOAI3333999 VIT-1239

MW: 349.44

HPLCMS: [m/z]: 350

FIG. 18 shows the result.

### Compound according to Example 19

### 7-[(Benzyl-methyl-amino)-pyridin-4-yl-methyl]-quinolin-8-ol

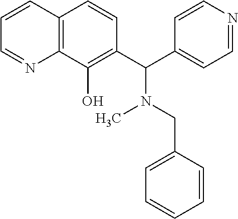

The preparation was carried out analogously to the method described for Example Compound 17 using:
8-hydroxyquinoline (250 mg, 2.0 mmol)
4-pyridinecarboxaldehyde (194 mg, 2.0 mmol)
and N-methylbenzylamine (376 mg, 3.0 mmol)

Purification was carried out by means of recrystallization from methanol to give Example Compound 19 (200 mg, 33%).

EOAI3333816 VIT-1229

MW: 355.44

HPLCMS: [m/z]: 356

FIG. 19 shows the result.

### Compound according to Example 20

### 7-[(2,6-Dimethyl-morpholin-4-yl)-(4-fluoro-phenyl)-methyl]-quinolin-8-ol

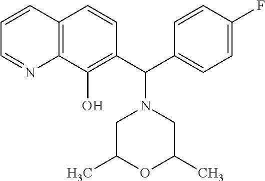

The preparation was carried out analogously to the method described for Example Compound 17 using:
8-hydroxyquinoline (250 mg, 2.0 mmol)
4-fluorobenzaldehyde (224 mg, 2.0 mmol)
and 2,6-dimethylmorpholine (357 mg, 3.0 mmol)

Purification was carried out by means of preparative HPLC to give Example Compound 20 (15 mg, 2%).

EOAI3334000 VIT-1238

MW: 366.44

HPLCMS: [m/z]: 367

FIG. 20 shows the result.

### Compound according to Example 21

### 7-(Morpholin-4-yl-thiophen-2-yl-methyl)-quinolin-8-ol

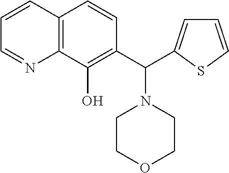

8-Hydroxyquinoline (259 mg, 1.78 mmol), 2-thiophenecarboxyaldehyde (200 mg, 1.78 mmol) and morpholine (155 mg, 1.78 mmol) were dissolved in anhydrous toluene (5 ml) under a nitrogen atmosphere. The mixture was heated at 110° C. for 18 hours. After cooling, the mixture was concentrated in vacuo and the crude material obtained in this way was dissolved in MC (methylene chloride) and washed with water and brine. The organic phase was dried by means of Na₂SO₄ and concentrated in vacuo.

Purification was carried out by means of column chromatography with methanol/MC (2%) as the eluent to give Example Compound 21 (40 mg, 6%).

EOAI3330057 VIT-1087

MW: 326.42

HPLC: [m/z]: 239.95 (M+-morpholine) structure confirmed by means of NMR.

FIG. 21 shows the result.

### Compound according to Example 22

### 7-Morpholin-4-yl-methyl-quinolin-8-ol

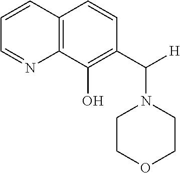

8-Hydroxyquinoline (3.0 g, 20.7 mmol), formaldehyde (37% aqueous solution, 2.4 ml) and morpholine (1.8 ml, 20.7 mmol) were dissolved in ethanol (35 ml). The mixture was stirred at room temperature for 24 hours. The mixture was concentrated in vacuo.

Purification was carried out by means of column chromatography with methanol/MC (0-10%) as the eluent to give Example Compound 22 (237 mg, 5%).

EOAI3327182 VIT-1040

MW: 244.29

HPLCMS: [m/z]: 245

FIG. 22 shows the result.

### Compound according to Example 23

### 7-Morpholin-4-ylmethyl-8-(3,4,5-trimethoxybenzyloxy)-quinoline

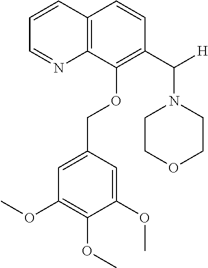

A solution of 7-morpholin-4-ylmethylquinolin-8-ol (53 mg, 0.21 mmol; obtainable e.g. according to Preparation Example 22) in DMF (1 ml) was treated with potassium carbonate (58 mg, 0.42 mmol) and 3,4,5-trimethoxybenzyl chloride (69 mg, 0.32 mmol). The mixture was stirred at room temperature for 18 hours. The solution was diluted with ethyl acetate (5 ml) and washed with water and brine. The resulting organic phase was dried by means of MgSO₄ and concentrated in vacuo.

Purification was carried out by means of column chromatography with methanol/MC (2%) as the eluent to give Example Compound 23 (40 mg, 43%).

EOAI3332897 VIT-1171

MW: 424.50

HPLCMS: [m/z]: 425

FIG. 23 shows the result.

### Compound according to Example 24

### 4-[(8-Hydroxy-quinolin-7-yl)-morpholin-4-yl-methyl]-benzamide

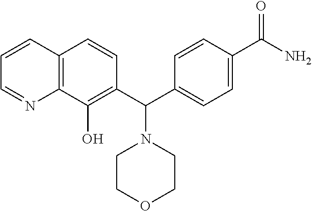

4-[(8-Hydroxy-quinolin-7-yl)-morpholin-4-yl-methyl]-benzonitrile (40 mg, 0.12 mmol; obtainable e.g. according to Preparation Example 10), was dissolved in TEA (triethylamine) (1.6 ml) and conc. sulfuric acid (0.4 ml) and the mixture was stirred for 2 days. The reaction was interrupted by addition of ice. The mixture obtained in this way was neutralized with saturated NaHCO₃ and the aqueous phase was extracted with MC. The resulting organic phase was dried by means of MgSO₄ and concentrated in vacuo.

The crude material obtained in this way was dried in a vacuum oven at 40° C. to give Example Compound 24 (24 mg, 57%).

EOAI3333473 VIT-1194

MW: 363.41

HPLCMS: [m/z]: 364

FIG. 24 shows the result.

### Compound according to Example 25

### 5-Chloro-7-(morpholin-4-yl-pyridin-2-yl-methyl)-quinolin-8-ol

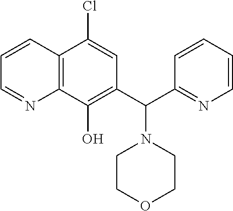

EOAI3327176 VIT-1045

MW: 355.83

Manufacturer: Life Chemicals

HPLCMS: [m/z]: 356

FIG. 25 shows the result.

### Compound according to Example 26

### 4-[(8-Hydroxy-quinolin-7-yl)-pyridin-2-yl-methyl]-piperazine-1-carboxylic acid ethyl ester

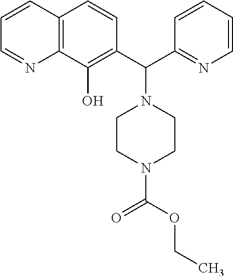

EOAI3327177 VIT-1046

MW: 392.46

Manufacturer: Life Chemicals

HPLCMS: [m/z]: 393

FIG. 26 shows the result.

### Compound according to Example 27

### 7-(Piperidin-1-yl-pyridin-2-yl-methyl)-quinolin-8-ol

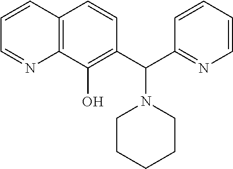

EOAI3327178 VIT-1047

MW: 319.41

Manufacturer: Life Chemicals

HPLCMS: [m/z]: 320

FIG. 27 shows the result.

### Compound according to Example 28

### 7-[(4-Fluoro-phenyl)-morpholin-4-ylmethyl-quinolin-8-ol

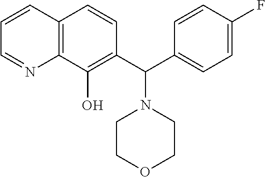

EOAI3327179 VIT-1048

MW: 338.39

Manufacturer: Life Chemicals

HPLCMS: [m/z]: 339

FIG. 28 shows the result.

### Compound according to Example 29

### 7-(Morpholin-4-yl-pyridin-3-yl-methyl)-quinolin-8-ol

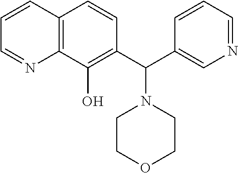

EOAI3327242 VIT-1055

MW: 321.38

Manufacturer: Life Chemicals

HPLCMS: [m/z]: 322

FIG. 29 shows the result.

### Compound according to Example 30

### 7-(Morpholin-4-yl-pyridin-4-yl-methyl)-quinolin-8-ol

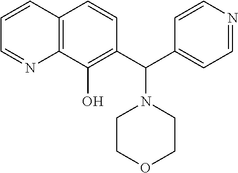

EOAI3327243 VIT-1056

MW: 321.38

Manufacturer: Life Chemicals

HPLCMS: [m/z]: 322

FIG. 30 shows the result.

### Compound according to Example 31

### 5-Chloro-7-morpholin-4-ylmethyl-quinolin-8-ol

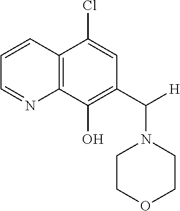

EOAI3284741 VIT-1057

MW: 278.74

Manufacturer: Life Chemicals

HPLCMS: [m/z]: 279

FIG. 31 shows the result.

### Compound according to Example 32

### 5-Chloro-7-(piperidin-1-yl-pyridin-2-yl-methyl)-quinolin-8-ol

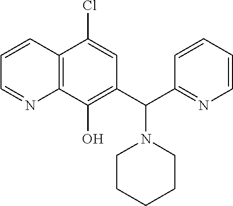

EOAI3327244 VIT-1058

MW: 353.85

Manufacturer: Life Chemicals

HPLCMS: [m/z]: 354

FIG. 32 shows the result.

### Compound according to Example 33

### 5-Chloro-7-[(2,6-dimethyl-morpholin-4-yl-pyridin-2-yl-methyl]-quinolin-8-ol

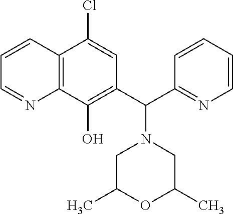

EOAI3327245 VIT-1059

MW: 383.88

Manufacturer: Enamine

HPLCMS: [m/z]: 383

FIG. 33 shows the result.

### Compound according to Example 34

### 7-[(4-Benzyl-piperazin-1-yl)-pyridin-2-yl-methyl]quinolin-8-ol

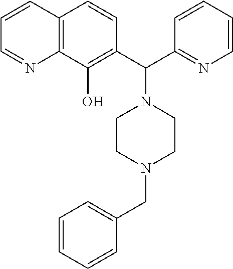

EOAI3327246 VIT-1060

MW: 410.52

Manufacturer: Enamine

HPLCMS: [m/z]: 411

FIG. 34 shows the result.

### Compound according to Example 35

### 7-[(4-Methyl-piperazin-1-yl)-pyridin-2-yl-methyl]-quinolin-8-ol

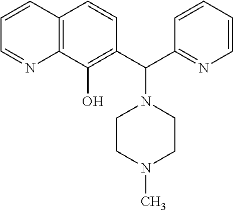

EOAI3328311 VIT-1064

MW: 334.42

Manufacturer: Enamine

HPLCMS: [m/z]: 335

FIG. 35 shows the result.

### Compound according to Example 36

### 7-{[4-(2-Fluoro-phenyl)-piperidin-1-yl]-pyridin-2-yl-methyl}-quinolin-8-ol

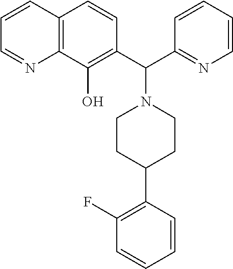

EOAI3328312 VIT-1065

MW: 413.5

Manufacturer: Enamine

HPLCMS: [m/z]: 415

FIG. 36 shows the result.

### Compound according to Example 37

### 7-[Pyridin-2-yl-(4-pyridin-2-yl-piperazin-1-yl)-methyl]-quinolin-8-ol

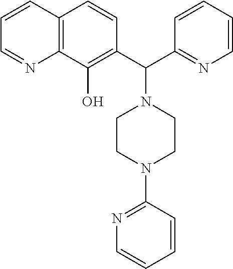

EOAI3328313 VIT-1066

MW: 397.48

Manufacturer: Enamine

HPLCMS: [m/z]: 398

FIG. 37 shows the result.

### Compound according to Example 38

### 5,7-Bis-morpholin-4-ylmethyl-quinolin-8-ol

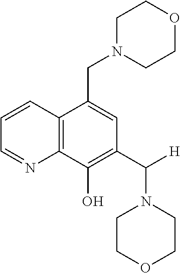

EOAI3328314 VIT-1067

MW: 343.43

Manufacturer: ChemBridge

HPLCMS: [m/z]: 344

FIG. 38 shows the result.

### Compound according to Example 39

### 5-Chloro-7-(morpholin-4-yl-thiophen-2-yl-methyl)-quinolin-8-ol

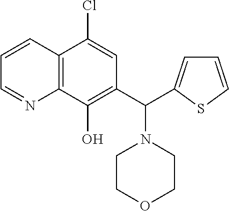

EOAI3328315 VIT-1063

MW: 360.87

Manufacturer: Enamine

HPLCMS: [m/z]: 291 (structure confirmed by means of NMR)

FIG. 39 shows the result.

### Compound according to Example 40

### 7-[(4-Chloro-phenyl)-morpholin-4-yl-methyl]-quinolin-8-ol

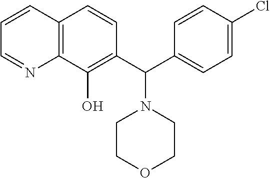

EOAI3328316 VIT-1062

MW: 354.84

Manufacturer: Enamine

HPLCMS: [m/z]: 355

FIG. 40 shows the result.

### Compound according to Example 41

### 7-[(4-Methoxy-phenyl)-morpholin-4-yl-methyl]-quinolin-8-ol

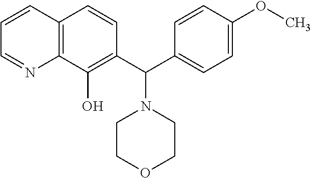

EOAI3328317 VIT-1061

MW: 350.42

Manufacturer: Enamine

HPLCMS: [m/z]: 351

FIG. 41 shows the result.

### DESCRIPTION OF THE FIGURES

FIG. 1: HPLC-MS of Example Compound 1

FIG. 2: HPLC-MS of Example Compound 2

FIG. 3: HPLC-MS of Example Compound 3

FIGS. 4a and 4b: HPLC-MS of Example Compound 4

FIG. 5: HPLC-MS of Example Compound 5

FIGS. 6a and 6b: HPLC-MS of Example Compound 6

FIG. 7: HPLC-MS of Example Compound 7

FIG. 8: HPLC-MS of Example Compound 8

FIG. 9: HPLC-MS of Example Compound 9

FIG. 10: HPLC-MS of Example Compound 10

FIG. 11: HPLC-MS of Example Compound 11

FIG. 12: HPLC-MS of Example Compound 12

FIG. 13: HPLC-MS of Example Compound 13

FIG. 14: HPLC-MS of Example Compound 14

FIG. 15: HPLC-MS of Example Compound 15

FIG. 16: HPLC-MS of Example Compound 16

FIG. 17: HPLC-MS of Example Compound 17

FIG. 18: HPLC-MS of Example Compound 18

FIG. 19: HPLC-MS of Example Compound 19

FIG. 20: HPLC-MS of Example Compound 20

FIG. 21: HPLC-MS of Example Compound 21

FIG. 22: HPLC-MS of Example Compound 22

FIG. 23: HPLC-MS of Example Compound 23

FIG. 24: HPLC-MS of Example Compound 24

FIG. 25: HPLC-MS of Example Compound 25

FIG. 26: HPLC-MS of Example Compound 26

FIG. 27: HPLC-MS of Example Compound 27

FIG. 28: HPLC-MS of Example Compound 28

FIG. 29: HPLC-MS of Example Compound 29

FIG. 30: HPLC-MS of Example Compound 30

FIG. 31: HPLC-MS of Example Compound 41

FIG. 32: HPLC-MS of Example Compound 32

FIG. 33: HPLC-MS of Example Compound 33

FIG. 34: HPLC-MS of Example Compound 34

FIG. 35: HPLC-MS of Example Compound 35

FIG. 36: HPLC-MS of Example Compound 36

FIG. 37: HPLC-MS of Example Compound 37

FIG. 38: HPLC-MS of Example Compound 38

FIG. 39: HPLC-MS of Example Compound 39

FIG. 40: HPLC-MS of Example Compound 40

FIG. 41: HPLC-MS of Example Compound 41

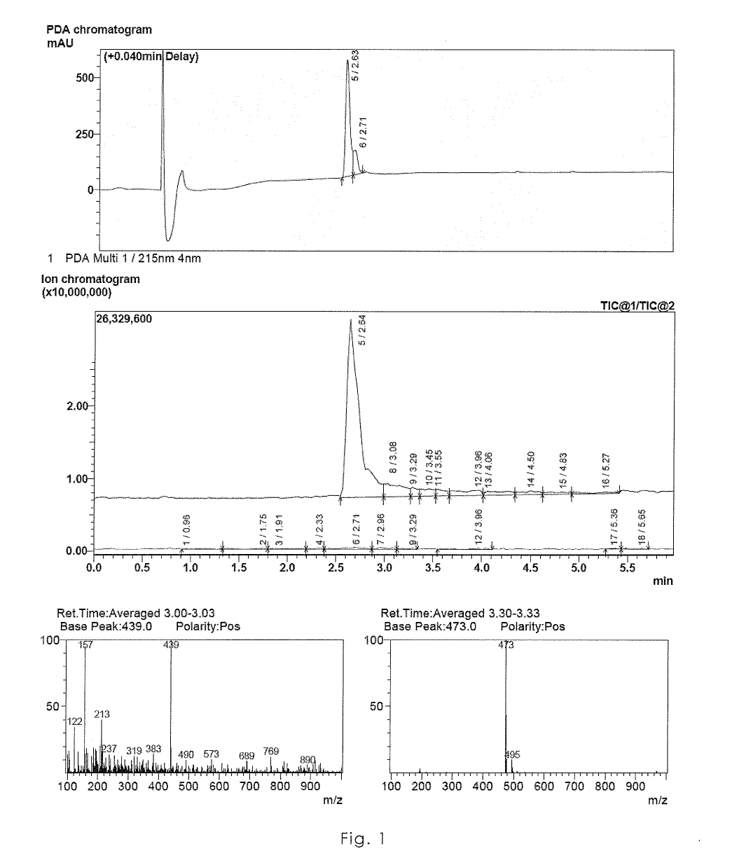

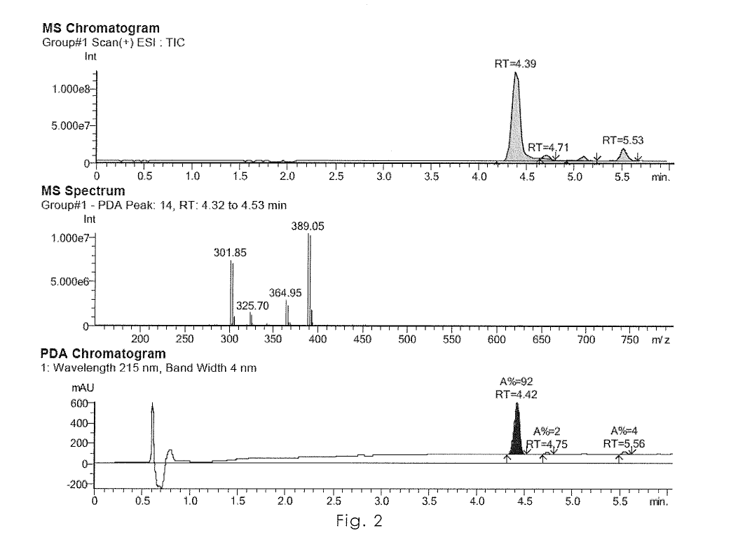

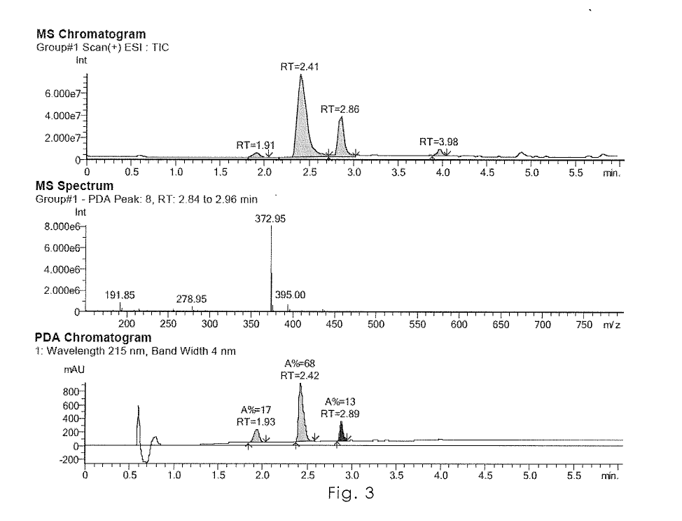

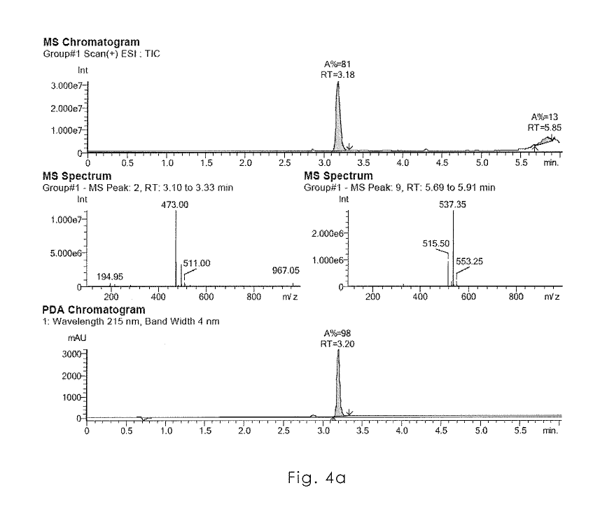

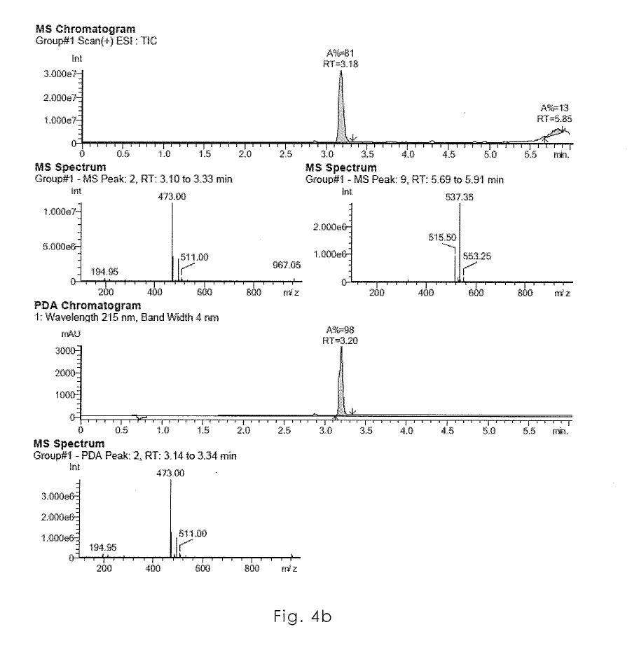

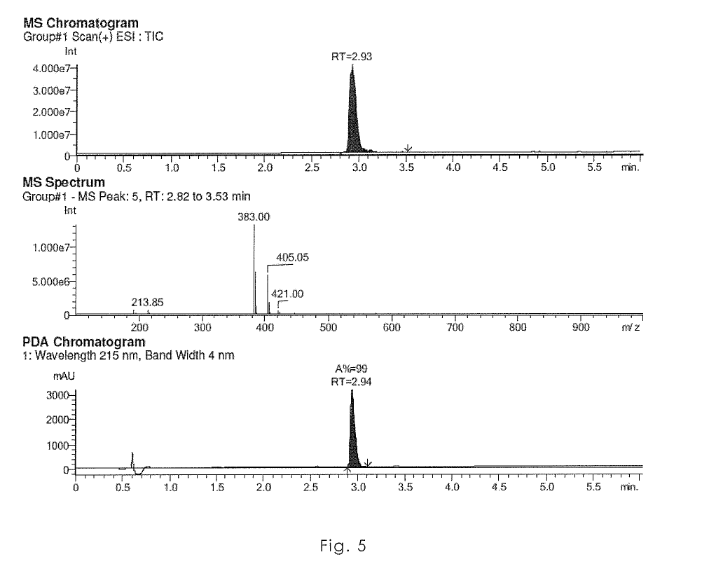

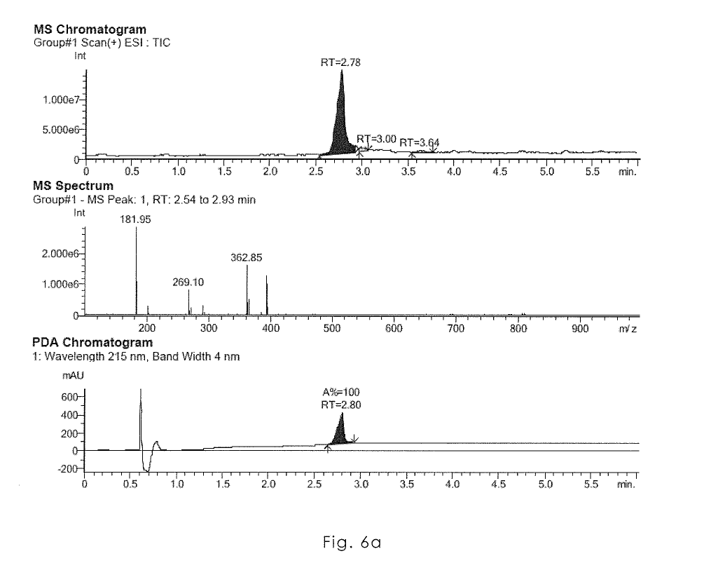

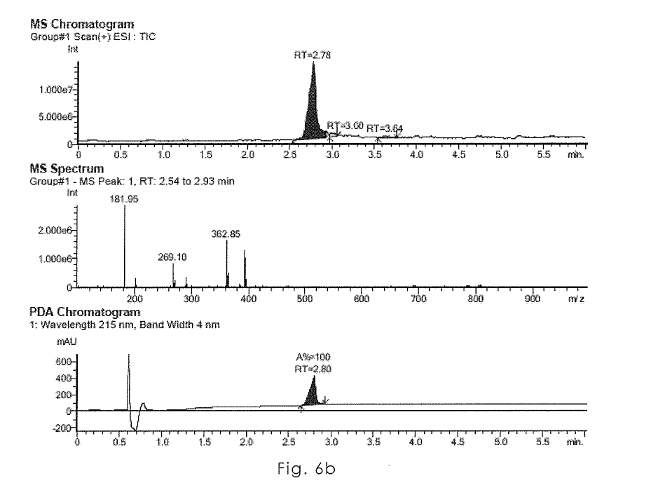

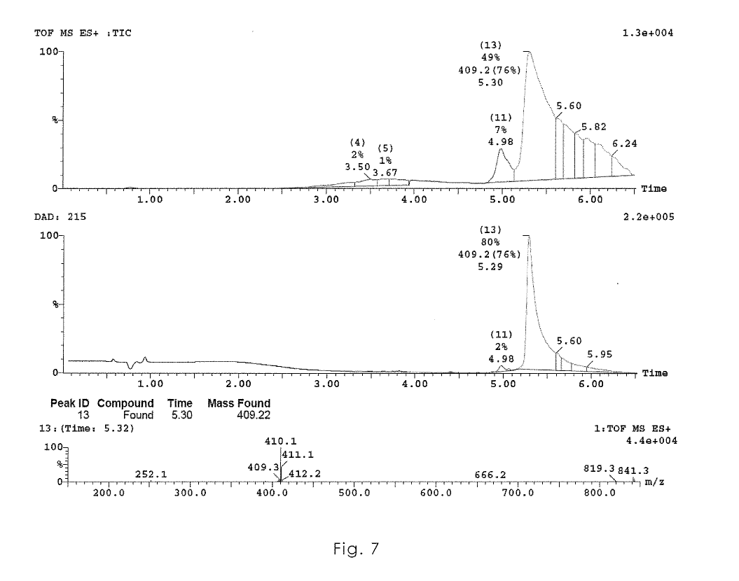

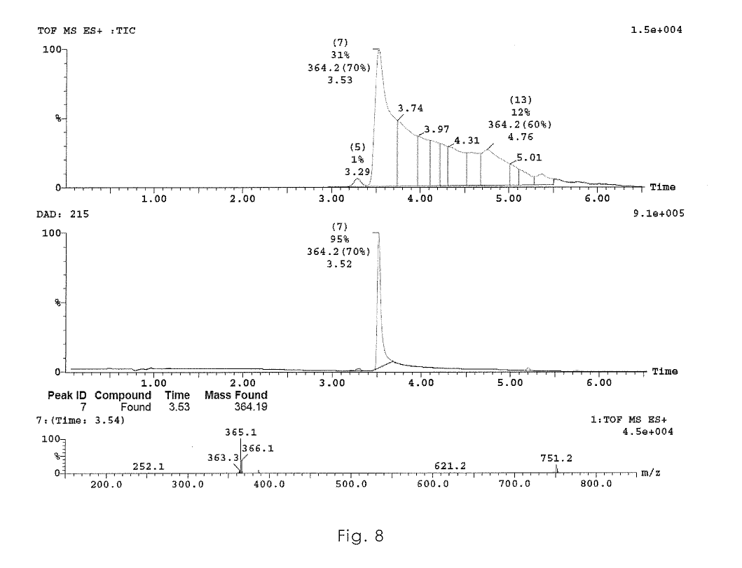

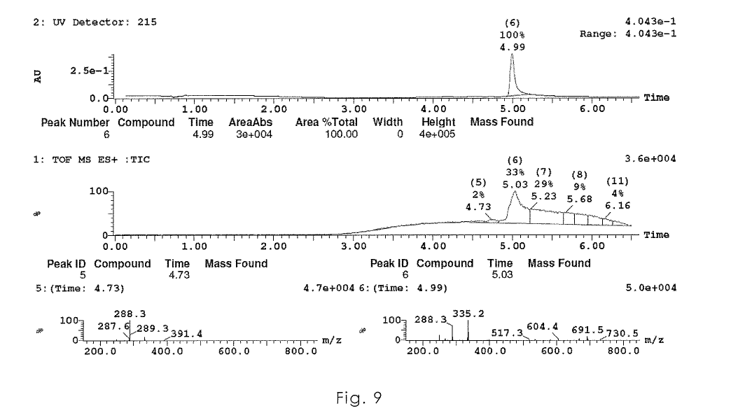

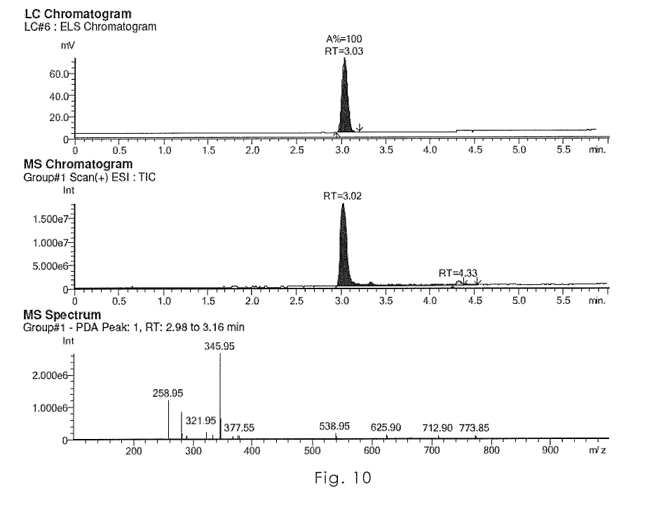

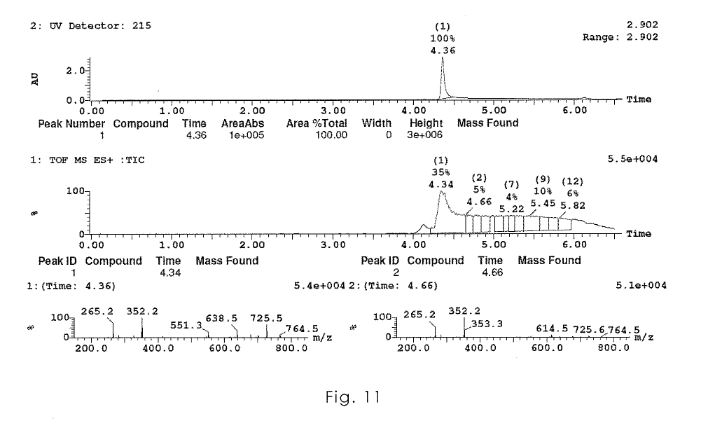

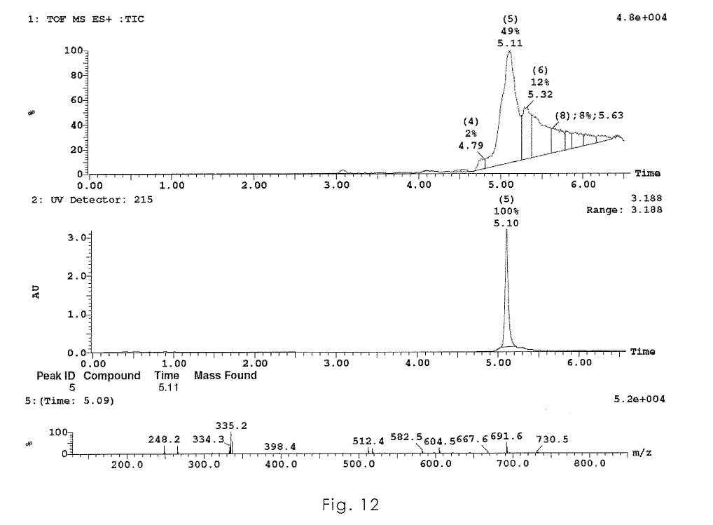

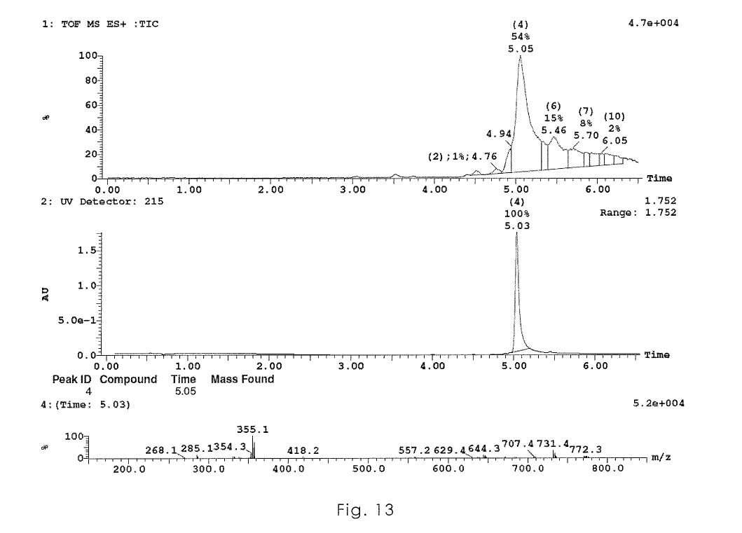

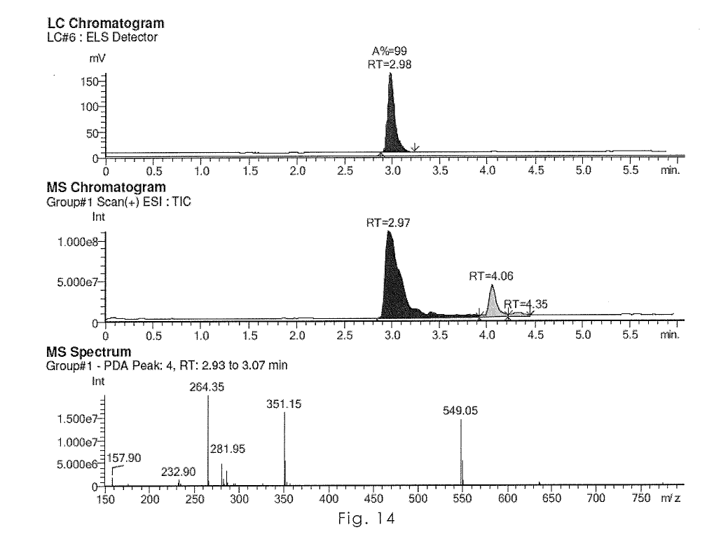

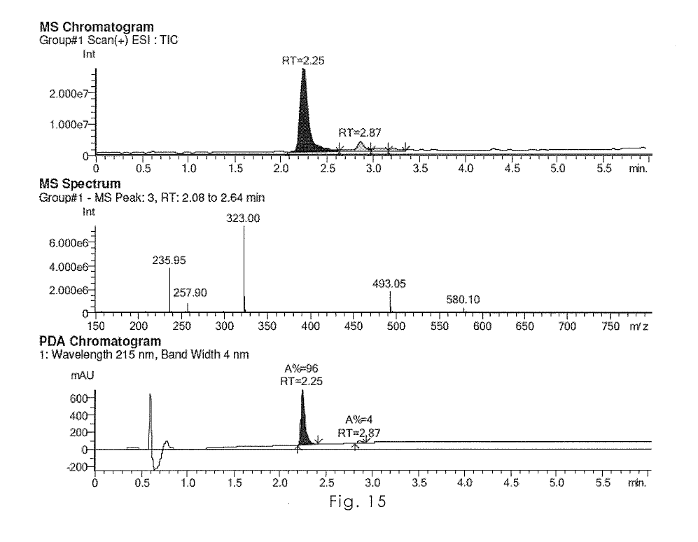

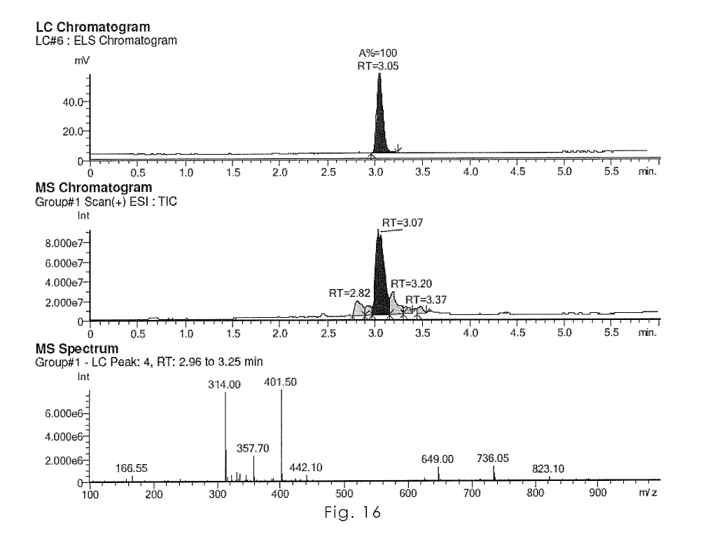

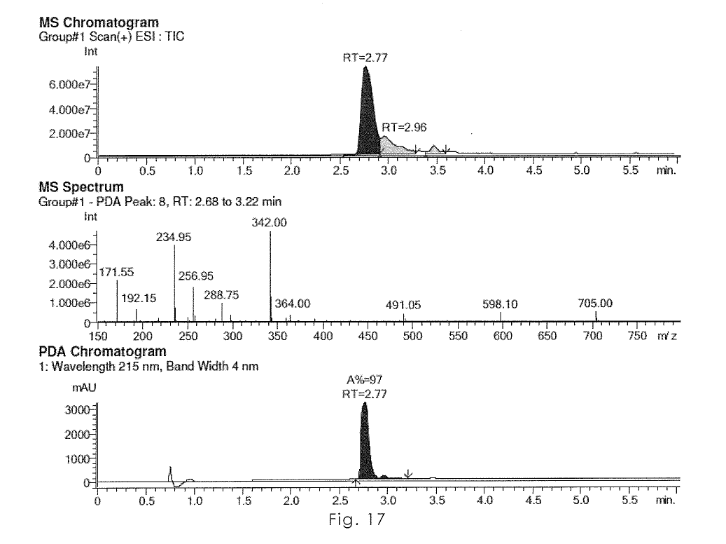

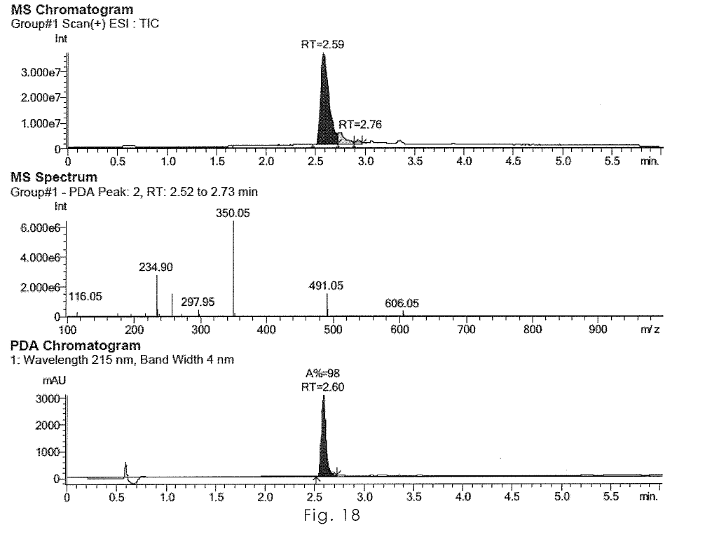

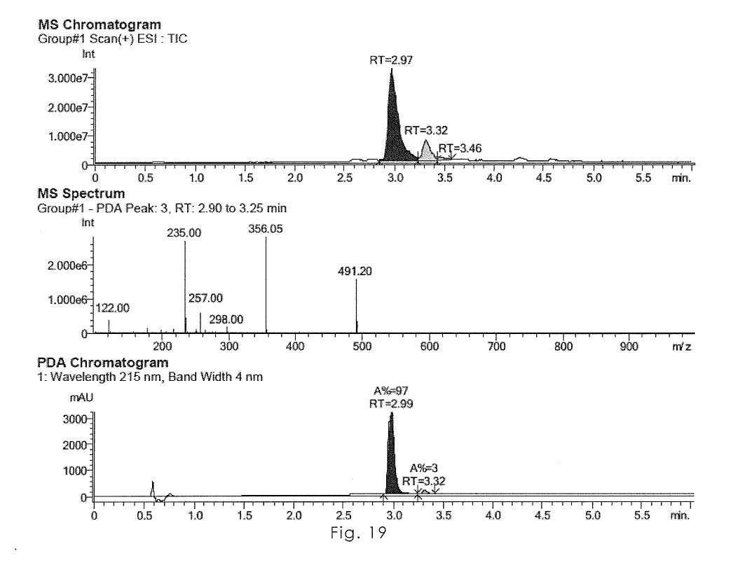

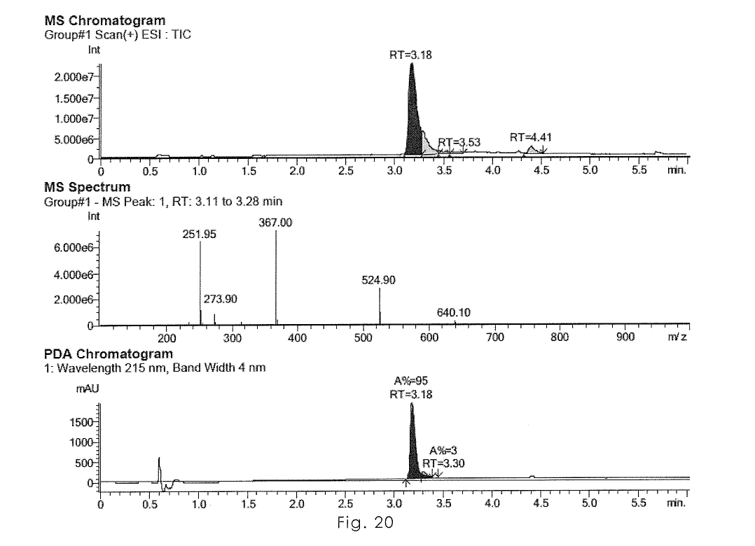

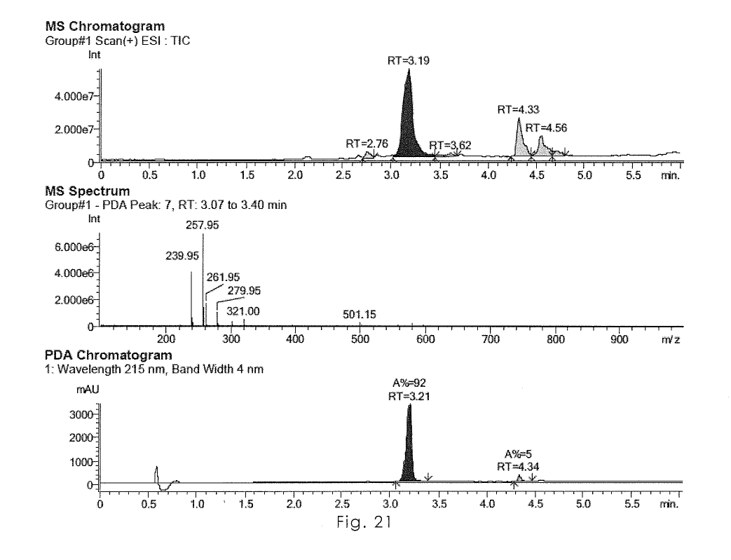

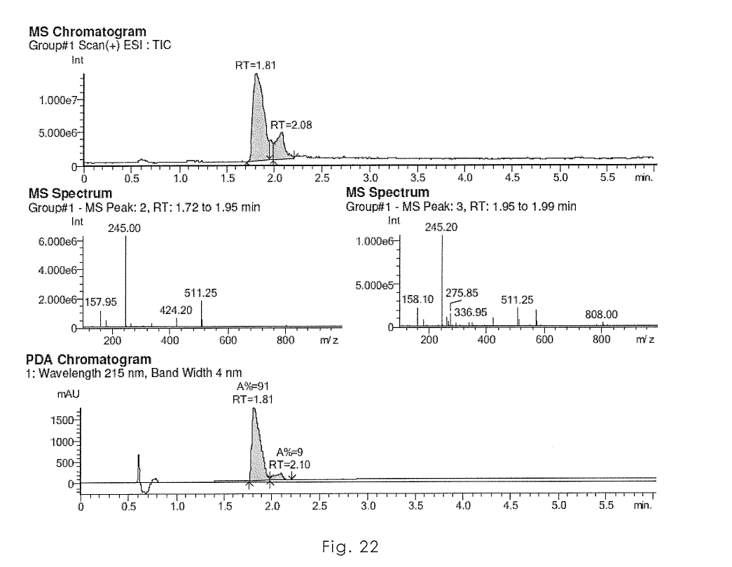

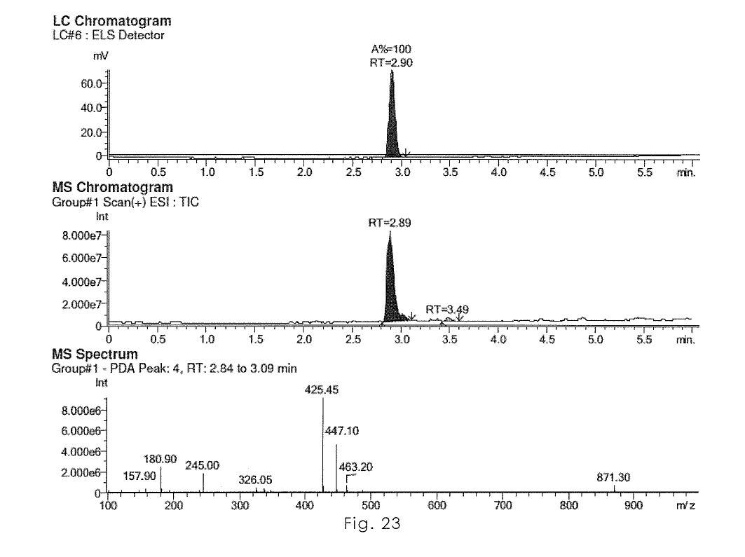

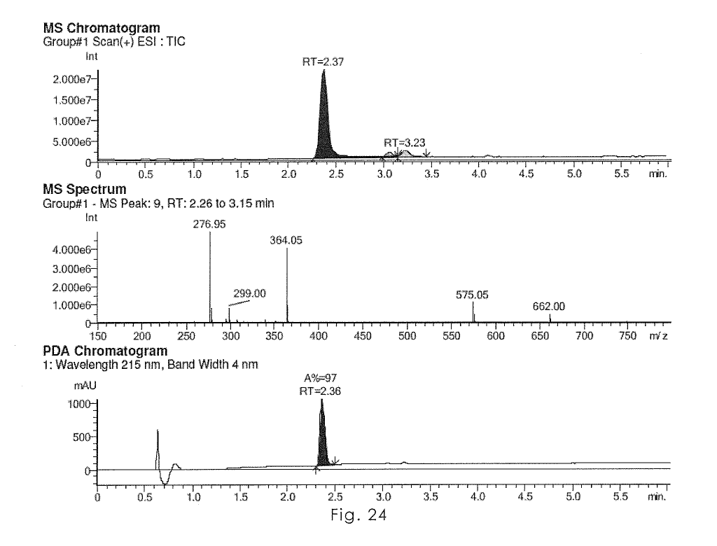

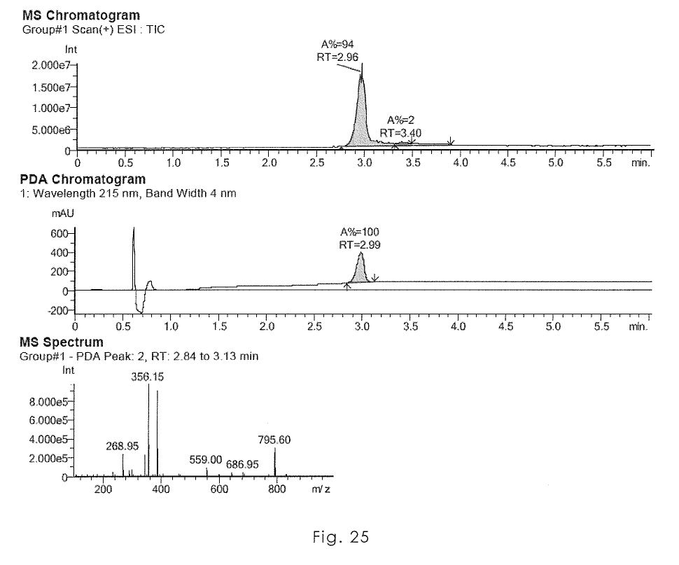

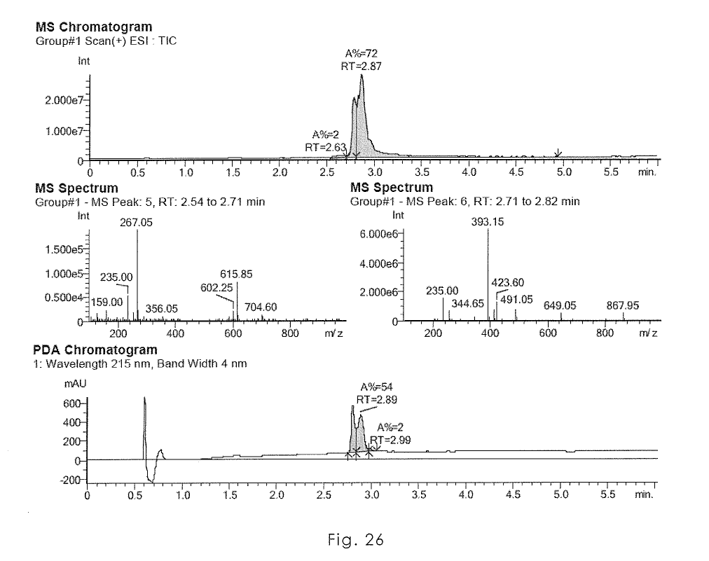

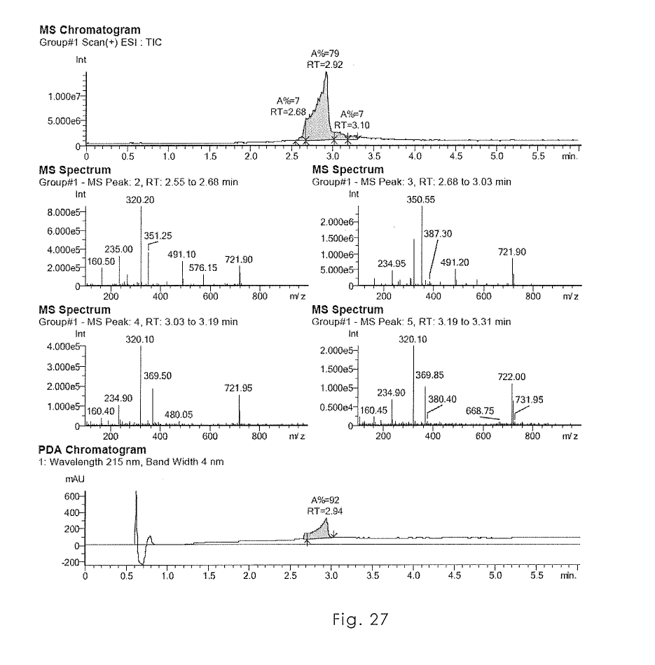

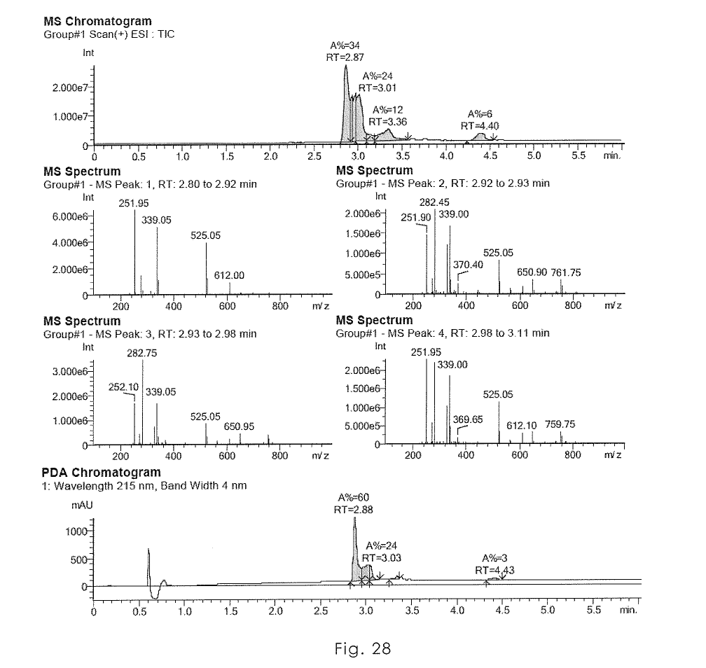

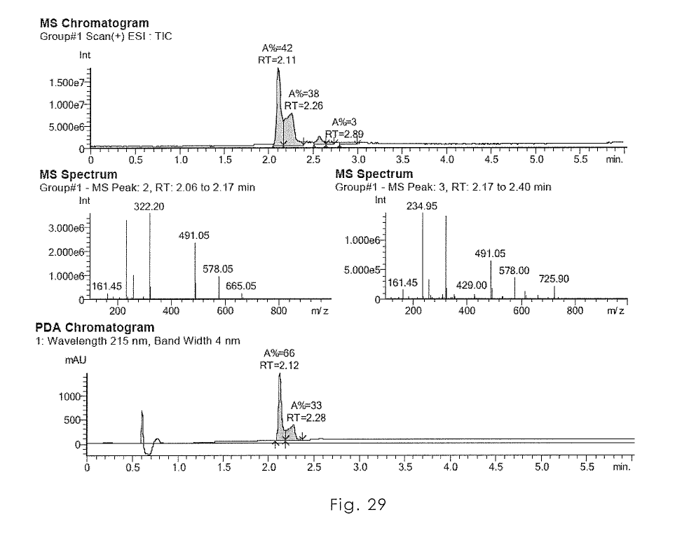

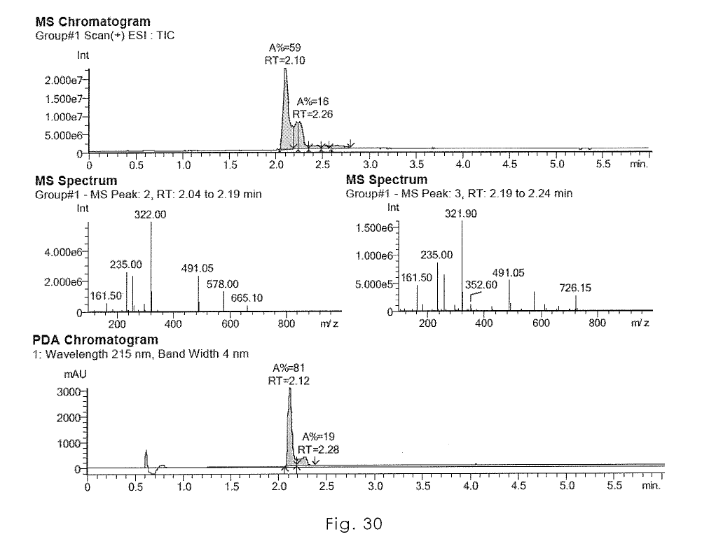

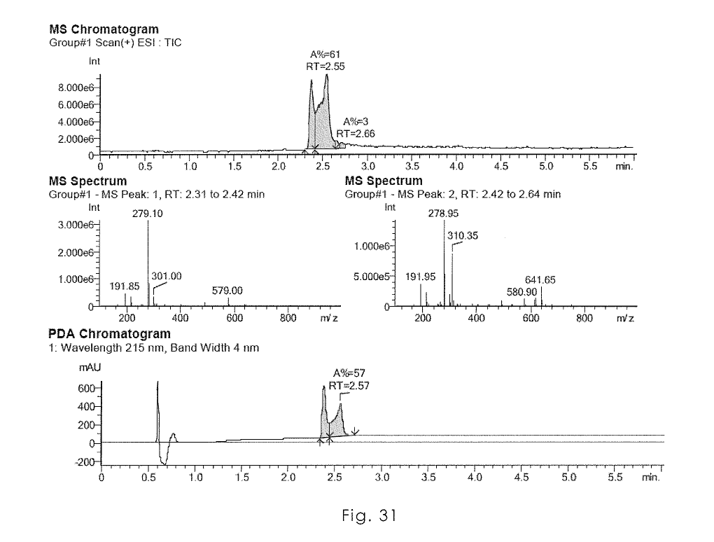

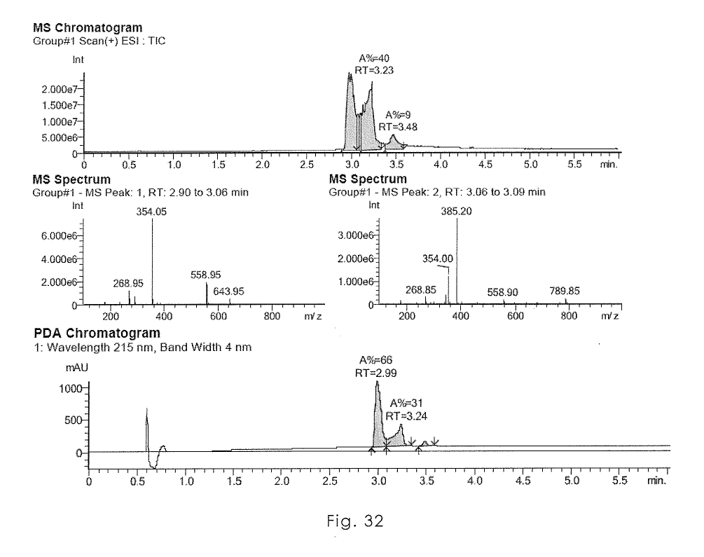

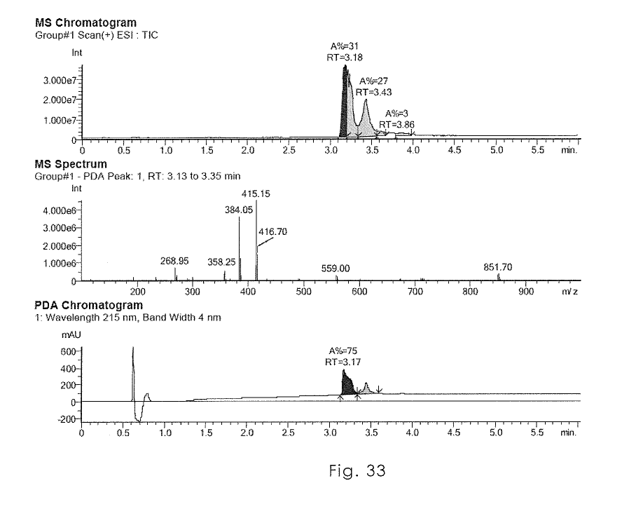

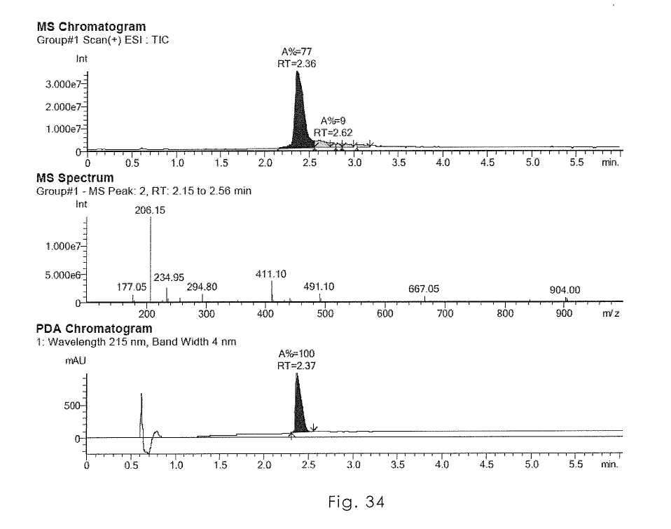

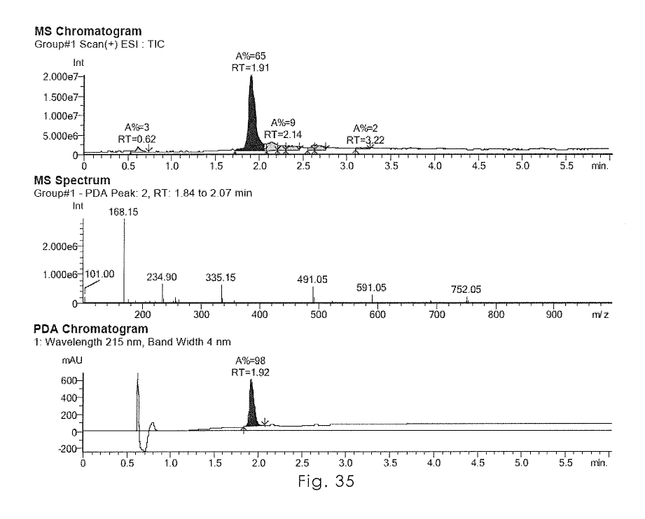

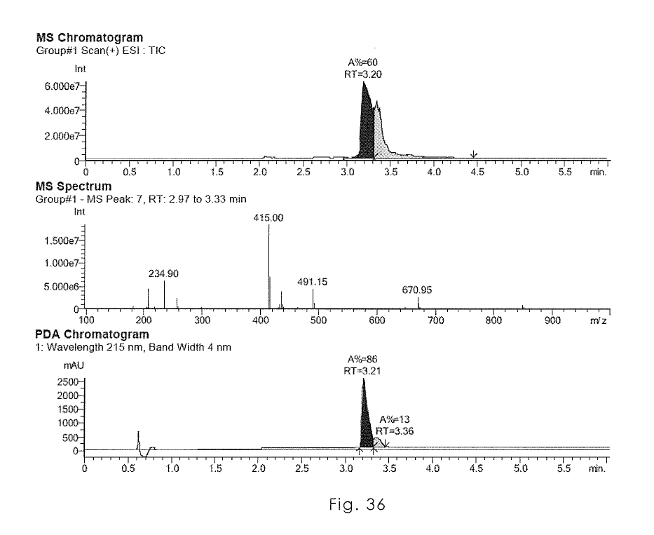

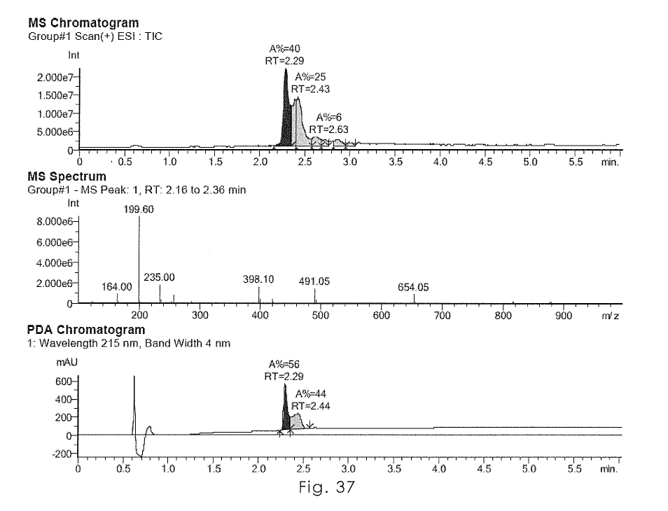

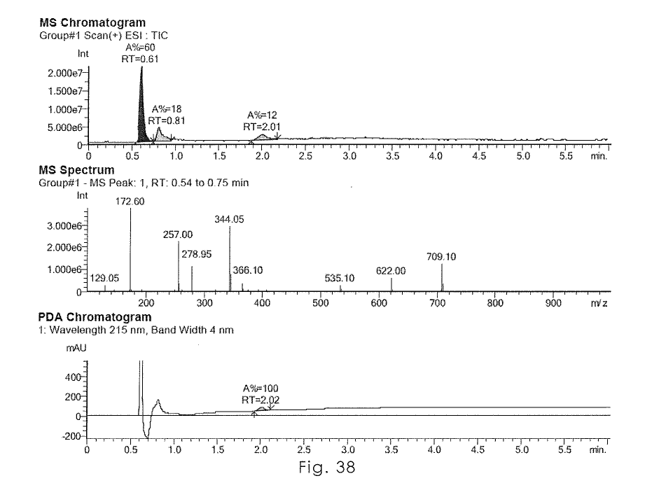

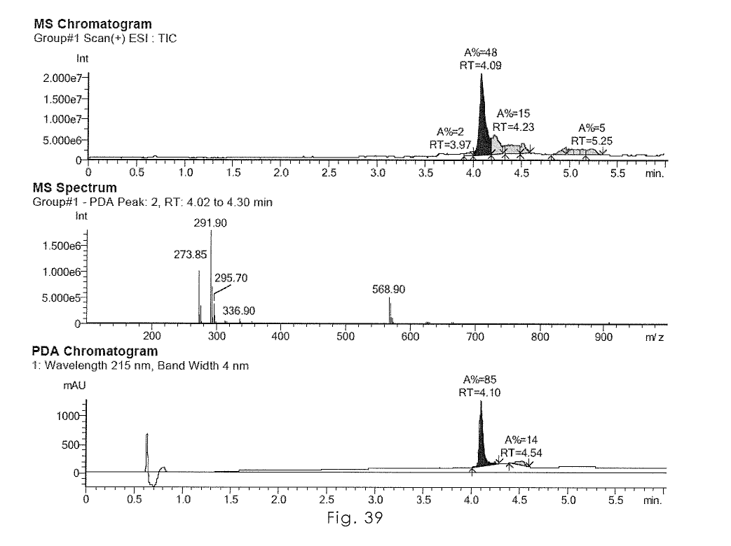

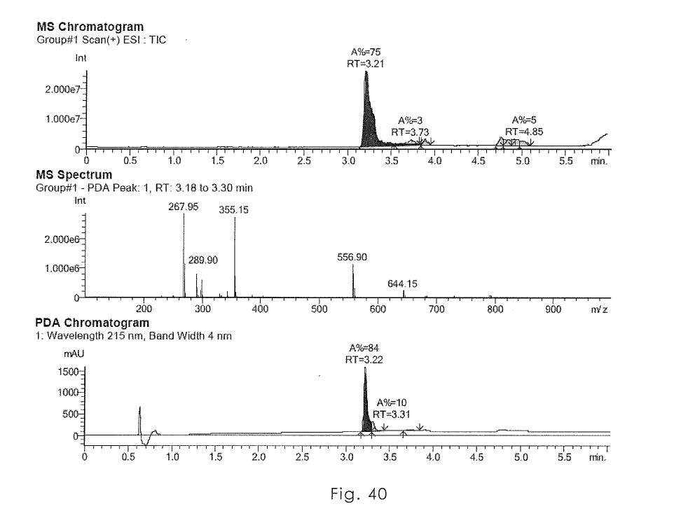

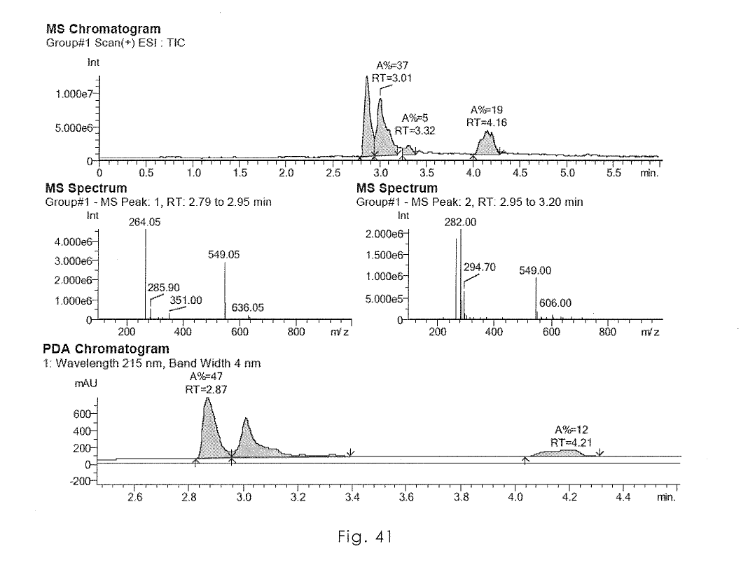

1. A method of treating at least one disorder including iron metabolism disorders, comprising, administering to a patient in need, a preparation including compounds of the general formula (I) 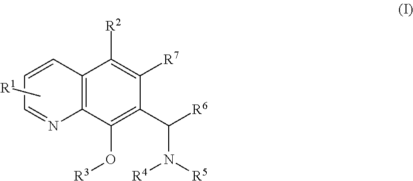
wherein
R¹, R² and R⁷ are identical or different and are each chosen from the group consisting of:
hydrogen,
hydroxyl,
halogen,
cyano,
nitro,
carboxyl,
sulfonic acid radical (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heteroaryl;
R⁶ is chosen from the group consisting of:
hydrogen,
optionally substituted alkyl,
optionally substituted alkenyl,
optionally substituted alkynyl,
optionally substituted aryl,
optionally substituted heteroaryl;
R³ is chosen from the group consisting of:
hydrogen,
optionally substituted alkyl,
optionally substituted alkenyl,
optionally substituted alkynyl,
optionally substituted acyl,
optionally substituted aryl,
optionally substituted heteroaryl; and
R⁴ and R⁵ are identical or different and are each chosen from the group consisting of:
hydrogen,
optionally substituted alkyl,
optionally substituted alkenyl,
optionally substituted alkynyl,
optionally substituted acyl,
optionally substituted aryl,
optionally substituted heteroaryl, or
wherein R⁴ and R⁵ together with the nitrogen atom to which they are bonded form a saturated or unsaturated, optionally substituted 5- to 8-membered ring which can optionally contain further hetero atoms;
or pharmaceutically acceptable salts thereof.

2. The method according to claim 1, wherein
R¹, R² and R⁷ are identical or different and are each chosen from the group consisting of:
hydrogen,
hydroxyl,
halogen,
optionally substituted alkyl,
optionally substituted alkoxy,
optionally substituted aryl,
optionally substituted heteroaryl;
R⁶ is chosen from the group consisting of:
hydrogen,
optionally substituted alkyl,
optionally substituted aryl,
optionally substituted heteroaryl;
R³ is chosen from the group consisting of:
hydrogen,
optionally substituted alkyl,
optionally substituted aryl,
optionally substituted heteroaryl;
optionally substituted acyl; and
R⁴ and R⁵ are identical or different and are each chosen from the group consisting of:
hydrogen,
optionally substituted alkyl,
optionally substituted aryl,
optionally substituted heteroaryl;
or R⁴ and R⁵ together with the nitrogen atom to which they are bonded form a saturated or unsaturated, optionally substituted 5- to 8-membered ring which can optionally contain further hetero atoms;
or pharmaceutically acceptable salts.

3. The method according to claim 1, wherein
R¹, R² and R⁷ are identical or different and are each chosen from the group consisting of:
hydrogen,
hydroxyl,
halogen,
optionally substituted alkyl,
optionally substituted alkoxy;
R⁶ is chosen from the group consisting of:
hydrogen,
optionally substituted alkyl,
optionally substituted aryl,
optionally substituted heteroaryl;
R³ is chosen from the group consisting of:
hydrogen,
optionally substituted alkyl,
optionally substituted acyl; and
R⁴ and R⁵ are identical or different and are each chosen from the group consisting of
hydrogen,
optionally substituted alkyl,
optionally substituted aryl,
optionally substituted heteroaryl,
or wherein R⁴ and R⁵ together with the nitrogen atom to which they are bonded form a saturated or unsaturated, optionally substituted 5- to 6-membered ring which can optionally contain further hetero atoms;
or pharmaceutically acceptable salts thereof.

4. The method according to claim 1, wherein
R¹, R² and R⁷ are identical or different and are each chosen from the group consisting of:
hydrogen,
hydroxyl,
halogen,
optionally substituted alkyl,
optionally substituted alkoxy;
R⁶ is chosen from the group consisting of:
hydrogen,
optionally substituted alkyl,
optionally substituted aryl,
optionally substituted heteroaryl;
R³ is chosen from the group consisting of:
hydrogen,
optionally substituted alkyl,
optionally substituted acyl; and
R⁴ and R⁵ are identical or different and are each chosen from the group consisting of:
hydrogen,
optionally substituted aryl,
optionally substituted heteroaryl,
or wherein R⁴ and R⁵ together with the nitrogen atom to which they are bonded form a saturated or unsaturated, optionally substituted 5- to 6-membered ring which can optionally contain further hetero atoms;
or pharmaceutically acceptable salts thereof.

5. The method according to claim 1, with the meanings:
R¹:
hydrogen;
R²:
hydrogen,
halogen or
optionally substituted alkyl, in particular or cycloalkyl-substituted alkyl;
R³:
hydrogen,
optionally substituted alkyl, or optionally substituted arylalkyl or optionally substituted heteroarylalkyl, or
optionally substituted acyl, or optionally substituted aroyl or optionally substituted heteroaroyl;
R⁴ and R⁵ are identical or different and denote:
hydrogen,
optionally substituted alkyl,
optionally substituted aryl,
optionally substituted heteroaryl, or
R⁴ and R⁵ together with the nitrogen atom to which they are bonded form a saturated or unsaturated, optionally substituted 6-membered ring which can contain further hetero atoms;
R⁶:
hydrogen,
optionally substituted aryl, or
optionally substituted heteroaryl; and
R⁷:
hydrogen;
or pharmaceutically acceptable salts thereof.

6. The method according to claim 1, with the meanings:
R¹:
hydrogen;
R²:
hydrogen or
halogen;
R³:
hydrogen, or
optionally substituted acyl, in particular optionally substituted aroyl or optionally substituted heteroaroyl;
R⁴ and R⁵ are identical or different and denote:
hydrogen,
optionally substituted aryl,
optionally substituted heteroaryl, or
R⁴ and R⁵ together with the nitrogen atom to which they are bonded form a saturated or unsaturated, optionally substituted 6-membered ring which can contain further hetero atoms;
R⁶:
hydrogen,
optionally substituted aryl, or
optionally substituted heteroaryl; and
R⁷:
hydrogen;
or pharmaceutically acceptable salts thereof.

7. The method according to claim 1, wherein
R¹ is hydrogen,
R² is chosen from
hydrogen,
chlorine or
morpholinylalkyl, such as morpholinylmethyl;
R³ is chosen from
hydrogen,
optionally substituted benzyl or
optionally substituted furoyl or optionally substituted benzoyl;
R⁴ and R⁵ are identical or different and denote:
hydrogen,
optionally substituted alkyl,
optionally substituted heteroaryl, or
R⁴ and R⁵ together with the nitrogen atom to which they are bonded form a saturated, optionally substituted 6-membered ring which contains one or no further hetero atom;
R⁶ denotes:
hydrogen,
optionally halogen-, alkyl-, alkoxy-, aminocarbonyl- or cyano-substituted phenyl or
optionally substituted pyridinyl, pyrazinyl, imidazolyl or thienyl; and
R⁷ is hydrogen,
or pharmaceutically acceptable salts thereof.

8. The method according to claim 1, wherein
R¹ is hydrogen,
R² is chosen from
hydrogen or
chlorine
R³ is chosen from
hydrogen, or
optionally substituted furoyl or optionally substituted benzoyl;
R⁴ and R⁵ are identical or different and denote:
hydrogen,
optionally substituted heteroaryl, or
R⁴ and R⁵ together with the nitrogen atom to which they are bonded form a saturated, optionally substituted 6-membered ring which contains a further hetero atom;
R⁶ denotes:
hydrogen,
optionally halogen-substituted phenyl or
pyridinyl; and
R⁷ is hydrogen,
or pharmaceutically acceptable salts thereof.

9. (canceled)

10. The method of claim 1, wherein the preparation includes compounds chosen from 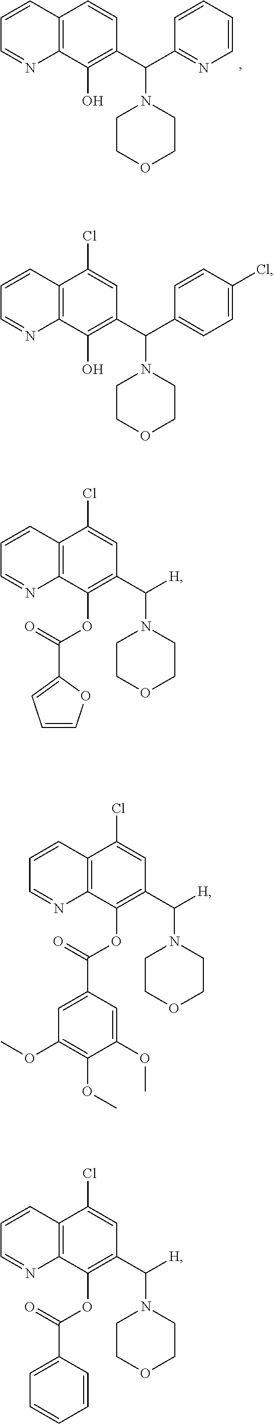 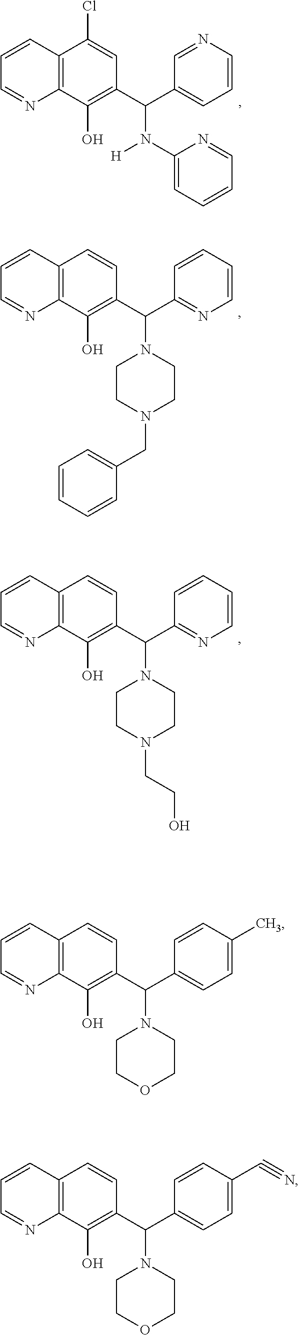 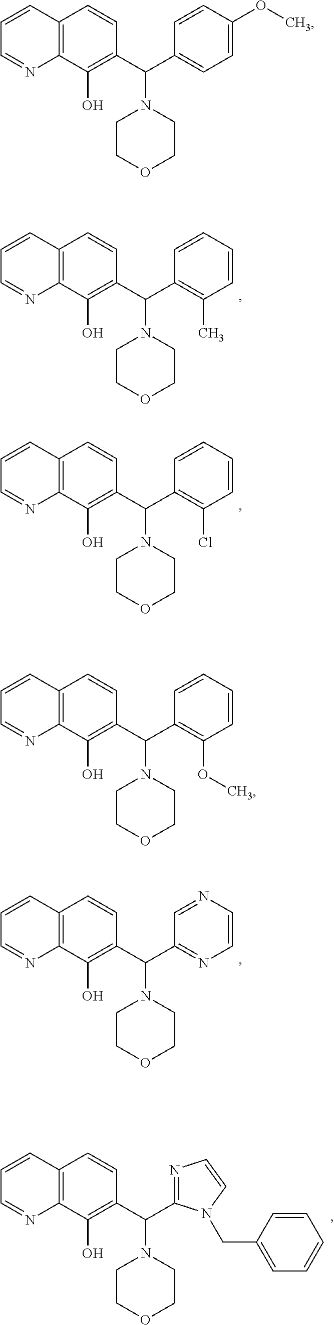 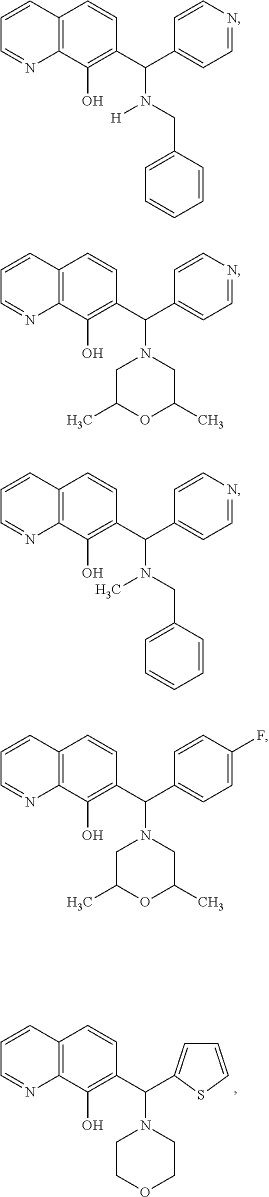 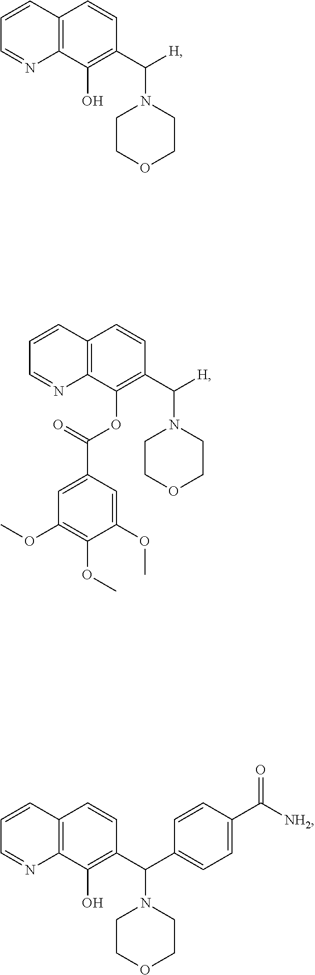 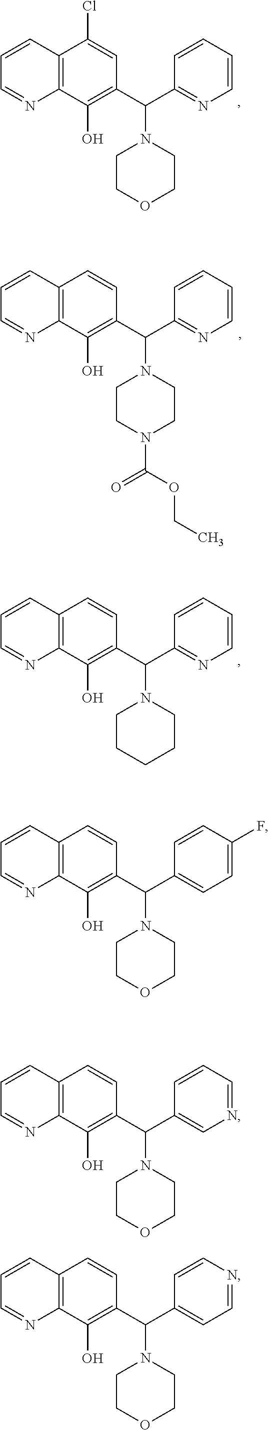 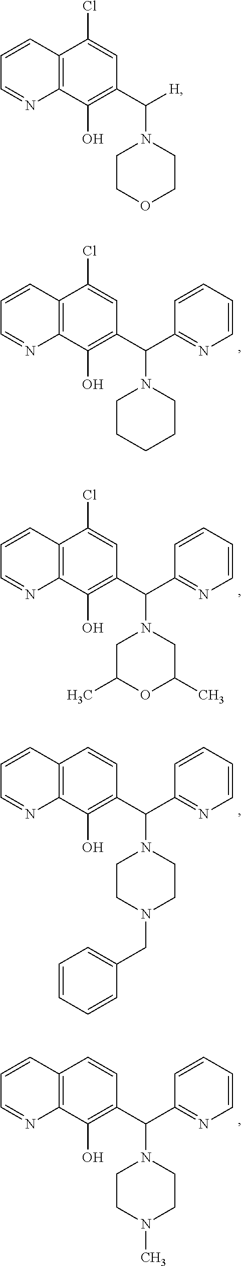 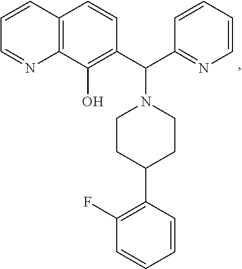 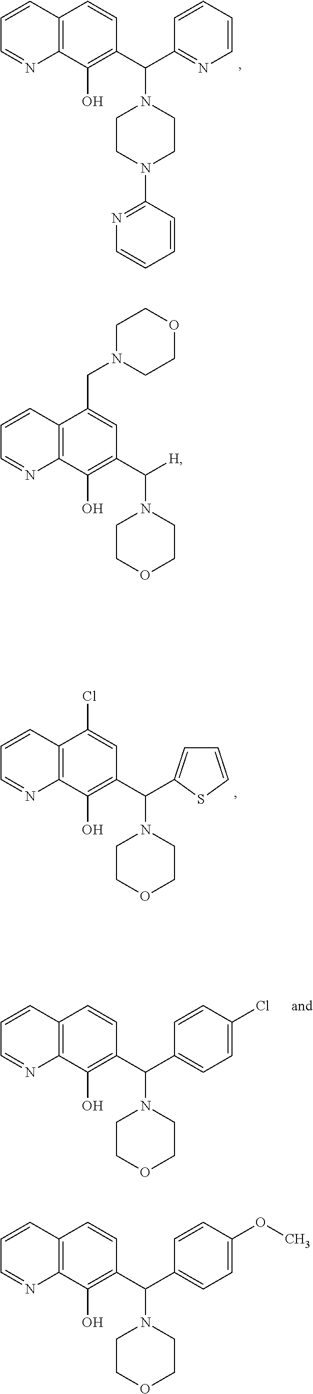
or pharmaceutically acceptable salts thereof.

11-14. (canceled)

15. The method according to claim 1 further comprising treating at least a second disorder, the second disorder selected from the group consisting of anaemias, anaemias with cancer, anaemia induced by chemotherapy, anaemia induced by inflammation, anaemias with congestive cardiac insufficiency anaemia with chronic renal insufficiency stage 3-5 anaemia induced by chronic inflammation anaemia with rheumatic arthritis anaemia with systemic lupus erythematosus and anaemia with inflammatory intestinal diseases.

16. The method of claim 1, further comprising administering one or more pharmaceutical carriers together with at least one of auxiliary substances and solvents.

17. The method of claim 1, wherein the preparation further comprises at least one further pharmaceutically active compound, wherein the pharmaceutically active compound is a compound for treatment of disorders in iron metabolism and the accompanying symptoms, wherein said pharmaceutically active compound is an iron-containing compound.

18-20. (canceled)

21. A method of preparing a medicament for oral or parental administration comprising combining:
(a) at least one compound of the general formula (I), 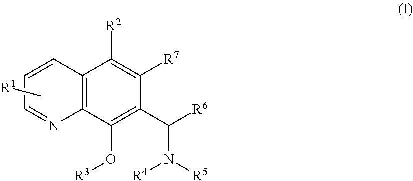
(b) at least one pharmaceutical carrier together with at least one of
(i) an auxiliary substance and
(ii) a solvent and
(c) an iron-containing compound for use in treating disorders in iron metabolism, wherein
R¹, R² and R⁷ are identical or different and are each chosen from the group consisting of:
hydrogen,
hydroxyl,
halogen,
cyano,
nitro,
carboxyl,
sulfonic acid radical (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heteroaryl;
R⁶ is chosen from the group consisting of:
hydrogen,
optionally substituted alkyl,
optionally substituted alkenyl,
optionally substituted alkynyl,
optionally substituted aryl,
optionally substituted heteroaryl;
R³ is chosen from the group consisting of:
hydrogen,
optionally substituted alkyl,
optionally substituted alkenyl,
optionally substituted alkynyl,
optionally substituted acyl,
optionally substituted aryl,
optionally substituted heteroaryl; and
R⁴ and R⁵ are identical or different and are each chosen from the group consisting of:
hydrogen,
optionally substituted alkyl,
optionally substituted alkenyl,
optionally substituted alkynyl,
optionally substituted acyl,
optionally substituted aryl,
optionally substituted heteroaryl, or
wherein R⁴ and R⁵ together with the nitrogen atom to which they are bonded form a saturated or unsaturated, optionally substituted 5- to 8-membered ring which can optionally contain further hetero atoms;
or pharmaceutically acceptable salts thereof.

22. A method of treating disorders in iron metabolism comprising administering:
(a) at least one compound of the general formula (I) 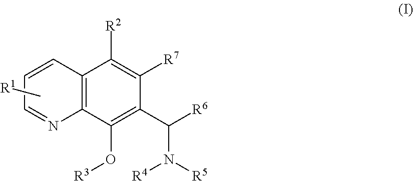
(b) at least one pharmaceutical carrier together with at least one of
(i) an auxiliary substance and
(ii) a solvent and
(c) an iron-containing compound for use in treating disorders in iron metabolism,
wherein
R¹, R² and R⁷ are identical or different and are each chosen from the group consisting of:
hydrogen,
hydroxyl,
halogen,
cyano,
nitro,
carboxyl,
sulfonic acid radical (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heteroaryl;
R⁶ is chosen from the group consisting of:
hydrogen,
optionally substituted alkyl,
optionally substituted alkenyl,
optionally substituted alkynyl,
optionally substituted aryl,
optionally substituted heteroaryl;
R³ is chosen from the group consisting of:
hydrogen,
optionally substituted alkyl,
optionally substituted alkenyl,
optionally substituted alkynyl,
optionally substituted acyl,
optionally substituted aryl,
optionally substituted heteroaryl; and
R⁴ and R⁵ are identical or different and are each chosen from the group consisting of:
hydrogen,
optionally substituted alkyl,
optionally substituted alkenyl,
optionally substituted alkynyl,
optionally substituted acyl,
optionally substituted aryl,
optionally substituted heteroaryl, or
wherein R⁴ and R⁵ together with the nitrogen atom to which they are bonded form a saturated or unsaturated, optionally substituted 5- to 8-membered ring which can optionally contain further hetero atoms;
or pharmaceutically acceptable salts thereof.
